(19) United States
(12) Patent Application Publication
(10) Pub. No.: US 2008/0045559 A1
(43) Pub. Date: Feb. 21, 2008

(54) TOCOPHEROL-MODIFIED THERAPEUTIC DRUG COMPOUNDS

(75) Inventors: Yuehua Zhang, Bothell, WA (US); Lynn C. Gold, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SONUS PHARMACEUTICALS, INC., Bothell, WA (US)

(21) Appl. No.: 11/843,568

(22) Filed: Aug. 22, 2007

Related U.S. Application Data
(63) Continuation-in-part of application No. 11/450,795, filed on Jun. 8, 2006.
Application No. 11/450,795 is a continuation of application No. 10/978,222, filed on Oct. 29, 2004, now Pat. No. 7,223,770.
(60) Provisional application No. 60/823,223, filed on Aug. 22, 2006.
(60) Provisional application No. 60/515,364, filed on Oct. 29, 2003.
(60) Provisional application No. 60/556,137, filed on Mar. 24, 2004.
(60) Provisional application No. 60/558,762, filed on Apr. 1, 2004.
(60) Provisional application No. 60/621,655, filed on Oct. 26, 2004.

(51) Int. Cl. A61K 31/355; A61K 31/437; A61P 35/00; C07D 311/72; C07D 471/22

(52) U.S. Cl. 514/283; 514/458; 546/48; 549/408

(57) ABSTRACT

Tocopherol-modified therapeutic drug compounds; emulsion, microemulsion, and micelle formulations that include the compounds; methods for making the compounds and formulations; methods for administering the compounds and formulations; and methods for treating conditions using the compounds and formulations.

### CROSS-REFERENCES TO RELATED APPLICATIONS

This application claims the benefit of U.S. Provisional Application No. 60/823,223, filed Aug. 22, 2006, and is a continuation-in-part of U.S. patent application Ser. No. 11/450,795, filed Jun. 8, 2006, which is a continuation of U.S. patent application Ser. No. 10/978,222, filed Oct. 29, 2004, now U.S. Pat. No. 7,223,770, which claims the benefit of U.S. Provisional Application No. 60/515,364, filed Oct. 29, 2003, U.S. Provisional Application No. 60/556,137, filed Mar. 24, 2004, U.S. Provisional Application No. 60/558,762, filed Apr. 1, 2004, and U.S. Provisional Application No. 60/621,655, filed Oct. 26, 2004, each of which is expressly incorporated herein by reference in its entirety.

### BACKGROUND OF THE INVENTION

The ability to administer biologically effective drugs that are poorly soluble in biocompatible solvents to mammals has been a major hurdle in the realm of pharmaceutical and medicinal chemistry. In particular, difficulties arise when an active drug is either insoluble in water or unstable in other biocompatible solvents.

One way to solubilize medicinal agents is to chemically modify them or conjugate them to another molecule to alter the solubility profile in a particular solvent. Conjugates of active drugs, often referred to as prodrugs, include chemical derivatives of biologically-active parent compounds that are converted into the parent compounds in vivo. The release of the active parent drug from the prodrug conjugate may occur as the result of processes such as hydrolysis or enzymatic cleavage. The rate of release is influenced by several factors, including the type of chemical bond joining the active parent drug to the conjugate moiety.

Incorporating a water-soluble moiety (e.g., polyethylene glycol, polyglutamate, or polymer) to increase solubility and circulation life of a drug has been investigated by others. The use of fatty acids to conjugate to active drugs for purposes of tumor targeting has also been investigated as a means of improving therapeutic index.

Many potent drugs, such as camptothecin and its analogues (e.g., 10-hydroxycamptothecin and 7-ethyl-10-hydroxycamptothecin), taxanes (e.g., paclitaxel, docetaxel), candesartan, amphotericin B, azathioprine, cyclosporine, entacapone, danazol, eletriptan, and bosentan, to name a few, are poorly soluble or have poor cell permeability. Solubility problems of potential therapeutic agents are common and often cause delays in drug development. Several technologies have been developed to facilitate the delivery of poorly soluble and insoluble compounds to patients. Examples of technologies specifically designed to solve solubility problems include complexing agents, nanoparticles, microemulsions, solubility enhancing formulations, prodrugs, and novel polymer systems.

Paclitaxel (see structure below), a natural product found in the inner bark of the Pacific Yew tree, is an example of an important chemotherapeutic agent with wide spectrum of activity against solid tumors, primarily breast, ovarian, colon and non-small cell lung cancer.

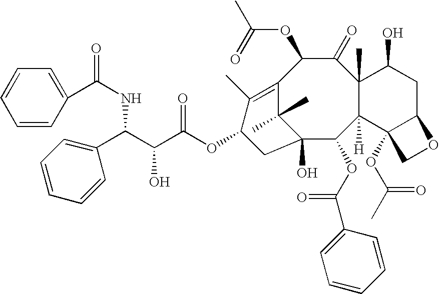

Paclitaxel exerts its antitumor activity by binding to tubulin and stabilizing microtubules and thus blocking cell mitosis. However, paclitaxel, like many other potent biologically active molecules, has very limited aqueous solubility.

Camptothecin (CPT) (see structure below) is another example of a poorly soluble and difficult to formulate anti-cancer drug.

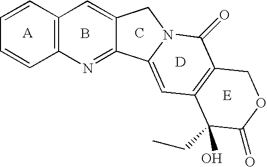

CPT is a quinoline-based alkaloid found in the bark of the Chinese camptotheca tree and the Asian nothapodytes tree. CPT includes four planar rings (ABCD) and one boat conformational ring (E). CPT has been found to have a broad spectrum of antitumor activity, especially in human solid tumors. However, the lactone (ring E) of camptothecin and its derivatives is quite labile in alkaline condition and physiological pH. The opening of this ring to form an acid salt or carboxylate species results in significant loss of anticancer activities. Efforts have been made since the early 1960s, when CPT was discovered by Wall and Wani, to improve upon the anti-cancer activities of camptothecin and its analogues, and to reduce unwanted toxicities. No successful formulation of camptothecin has been developed to date because of its poor solubility in both water and organic solvents. However, water-soluble analogues of camptothecin, irinotecan hydrochloride (CAMPTOSAR) and topotecan hydrochloride (HYCAMPTIN), have been developed and are the only camptothecin analogs currently approved by the Food and Drug Administration.

Recently, a vitamin E (α-tocopherol)-based emulsion formulation technology for paclitaxel drug delivery has been developed. In the formulation, paclitaxel is solubilized in α-tocopherol and formulated as an oil-in-water emulsion. However, while paclitaxel is soluble in α-tocopherol, the solubility of other active moieties (including camptothecin and other taxanes) in α-tocopherol is limited. Therefore, there continues to be a need for new methods, which are both safe and efficacious, of solubilizing and delivering poorly soluble active drug molecules.

### SUMMARY OF THE INVENTION

In one aspect, the present invention provides therapeutic drug compounds that have been modified to increase their lipophilicity. The compounds of the invention include a one or more therapeutic drug moieties and one or more lipophilic moieties. The therapeutic drug moiety is covalently coupled to the lipophilic moiety either directly or by a linker moiety. Methods for making the modified therapeutic drugs are also provided.

In one embodiment, compounds of the invention have formula (I):
T-O—C(═O)—R—C(═O)—O-D (I)

wherein

T is a tocopherol moiety derived from a tocopherol selected from the group consisting of:

(a) α-tocopherol,

(b) β-tocopherol,

(c) γ-tocopherol,

(d) δ-tocopherol,

(e) α-tocotrienol,

(f) β-tocotrienol,

(g) γ-tocotrienol, and

(h) δ-tocotrienol;

D is a therapeutic drug moiety derived from a therapeutic drug selected from the group consisting of:

(a) paclitaxel and its derivatives,

(b) docetaxel and its derivatives, and

(c) camptothecin and its derivatives; and

R is a divalent moiety selected from the group consisting of:

(a) substituted and unsubstituted alkyl,

(b) substituted and unsubstituted cycloalkyl, and

(c) substituted and unsubstituted aryl.

In one embodiment, the tocopherol moiety is selected from an α-tocopherol moiety and a δ-tocopherol moiety. In one embodiment, the tocopherol moiety is a dl-α-tocopherol moiety.

In one embodiment, the therapeutic drug moiety is selected from the group consisting of a 10-hydroxycamptothecin moiety and a 7-ethyl-10-hydroxycamptothecin moiety.

In one embodiment, the divalent moiety is substituted and unsubstituted alkyl.

In another embodiment, compounds of the invention have formula (II):

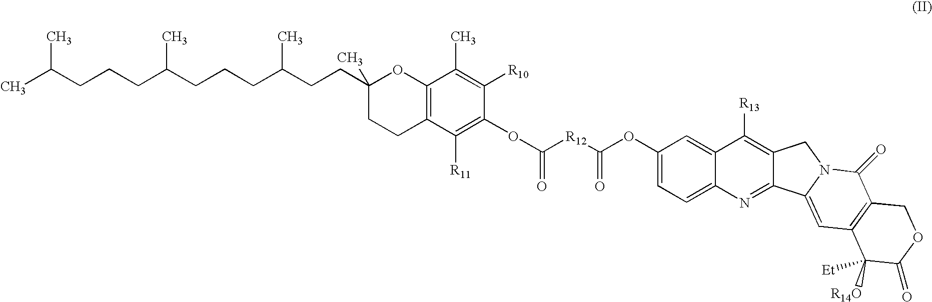

or a stereoisomer, tautomer, or pharmaceutically acceptable salt thereof,

wherein

R₁₀ is hydrogen or methyl;

R₁₁ is hydrogen or methyl;

R₁₂ is selected from the group consisting of:

(a) —(CH₂)ₙ—, where n is an integer from 3 to 12;

(b) —CH₂—CH(CH₃)—CH₂—;

(c) —CH₂—C(CH₂)ₘ—CH₂—, where m is an integer from 2 to 6;

(d) 1,3-phenylene;

(e) 1,4-phenylene;

(f) 1,2-cycloalkylene;

(g) 1,3-cycloalkylene; and

(h) 1,4-cycloalkylene;

R₁₃ is hydrogen or ethyl; and

R₁₄ is selected from the group consisting of hydrogen and —C(═O)—R₁₅, where R₁₅ is C1-C6 alkyl.

In one embodiment, R₁₀ and R₁₁ are methyl.

In one embodiment, R₁₀ and R₁₁ are hydrogen.

In one embodiment, R₁₂ is —(CH₂)ₙ— and n is an integer from 3 to 12. In one embodiment, R₁₂ is —(CH₂)ₙ— and n is 3.

In one embodiment, R₁₃ is ethyl.

In one embodiment, R₁₄ is hydrogen.

In one embodiment, the tocopherol moiety is a dl-α-tocopherol moiety.

In a further embodiment, compounds of the invention have formula (III):

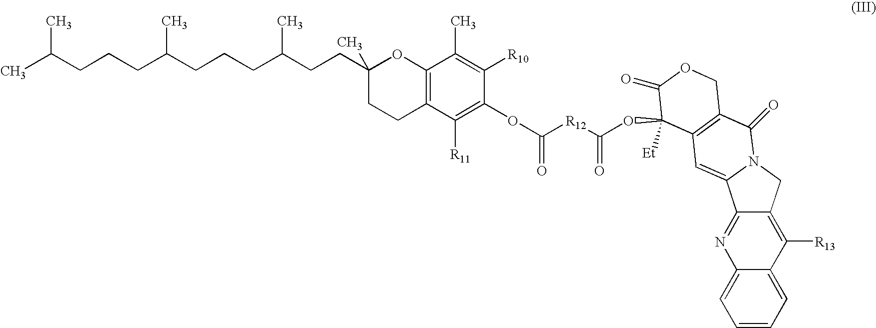

or a stereoisomer, tautomer, or pharmaceutically acceptable salt thereof,

wherein

R₁₀ is hydrogen or methyl;

R₁₁ is hydrogen or methyl;

R₁₂ is selected from the group consisting of:

(a) —(CH₂)ₙ—, where n is an integer from 3 to 12;

(b) —CH₂—CH(CH₃)—CH₂—;

(c) —CH₂—C(CH₂)ₘ—CH₂—, where m is an integer from 2 to 6;

(d) 1,3-phenylene;

(e) 1,4-phenylene;

(f) 1,2-cycloalkylene;

(g) 1,3-cycloalkylene; and

(h) 1,4-cycloalkylene; and

R₁₃ is hydrogen or ethyl.

In one embodiment, R₁₀ and R₁₁ are methyl.

In one embodiment, R₁₀ and R₁₁ are hydrogen.

In one embodiment, R₁₂ is —(CH₂)ₙ— and n is an integer from 3 to 12. In one embodiment, R₁₂ is —(CH₂)ₙ— and n is 3.

In one embodiment, R₁₃ is hydrogen.

In one embodiment, the tocopherol moiety is a dl-α-tocopherol moiety.

In another aspect of the invention, compositions that include the compounds of the invention are provided. In one embodiment, the composition includes a compound of the invention (e.g., a compound of formulas (I), (II), or (III)) and a pharmaceutically acceptable carrier. In certain embodiments, the compositions optionally one or more other therapeutic agents and/or a lipophilic medium. In one embodiment, the lipophilic medium includes a tocopherol. Methods for making the compositions are also provided.

In a further aspect, the invention provides emulsion and micelle formulations that include a compound of the invention (e.g., a compound of formulas (I), (II), or (III)). The emulsion formulation include an oil phase and an aqueous phase. The oil phase includes a compound of the invention and a lipophilic medium. The emulsion may be an oil-in-water emulsion or a water-in-oil emulsion. The micelle formulation includes a compound of the invention and an aqueous phase. Methods for making the emulsion and micelle formulations are also provided.

In other aspects, methods for administering the compounds of the invention (e.g., a compound of formulas (I), (II), or (III)) to a subject in need thereof, and methods for treating a condition treatable by administration of a therapeutically effective amount a compound of the invention (e.g., a compound of formulas (I), (II), or (III)) are also provided. In one embodiment, the condition treatable by administration of a compound of the invention is a solid tumor carcinoma. Representative treatable solid tumor carcinomas include breast, ovarian, pancreatic, colon, colorectal, non-small cell lung, and bladder carcinomas. In one embodiment, the invention provides a method for treating a solid tumor carcinoma comprising administering to a subject in need thereof an emulsion that includes (a) an oil phase comprising a compound of the invention (e.g., a compound of formulas (I), (II), or (III)) and a lipophilic medium; and (b) an aqueous phase. In one embodiment, the lipophilic medium includes a tocopherol.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 schematically illustrates reaction of d-α-tocopherol and a therapeutic drug containing a carboxyl group to provide a tocopherol-modified therapeutic drug compound;

FIG. 2 schematically illustrates tocopherol functionalization with a carbonyl chloride group (—C(═O)Cl) and a phosphoric chloride group (—P(═O)OR₁Cl), and reaction of the resulting acid chloride and an appropriately functionalized therapeutic drug compound to provide a tocopherol-modified therapeutic drug compound;

FIG. 3 schematically illustrates tocopherol functionalization with a dicarboxylic anhydride (succinic anhydride), and reaction of the resulting carboxylic acid and an appropriately functionalized therapeutic drug compound to provide a tocopherol-modified therapeutic drug compound;

FIG. 4 schematically illustrates the preparation of tocopherol succinate camptothecin;

FIG. 5 schematically illustrates the preparations of tocopherol succinate 10-hydroxycamptothecin and tocopherol succinate 7-ethyl-10-hydroxycamptothecin;

FIG. 6 schematically illustrates the preparation of 10,20-di(tocopherol succinate) 7-ethyl-10-hydroxycamptothecin;

FIG. 7 schematically illustrates the preparation of tocopherol succinate camptothecin containing a poly(ethylene oxide) group;

FIG. 8 schematically illustrates the preparation of tocopherol succinate paclitaxel;

FIG. 9 schematically illustrates the preparation of tocopherol succinate docetaxel;

FIG. 10 schematically illustrates the preparation of tocopherol terephthalate camptothecin;

FIG. 11 schematically illustrates the preparation of tocopherol cyclohexane-1,2-dicarboxylate 7-ethyl-10-hydroxycamptothecin;

FIG. 12 is a graph comparing the in vitro stability of the lactone form of camptothecin and of two representative tocopherol-modified therapeutic drug compounds of the invention (SN2300, tocopherol succinate camptothecin; and SN2310, tocopherol succinate 7-ethyl-10-hydroxycamptothecin);

FIG. 13 is a graph comparing GI₅₀ values reported by NCI for camptothecin, irinotecan hydrochloride (irinotecan), and topotecan hydrochloride (topotecan) with GI₅₀ values obtained for two representative tocopherol-modified therapeutic drug compounds of the invention (SN2300 and SN2310) for cell lines: H460, HCT-116, HT29, and OVCAR-3;

FIGS. 14A and 14B are graphs of concentration-time values after intravenous injection of 13.8 mg/kg of two representative tocopherol-modified therapeutic drug compounds of the invention (FIG. 14A, SN2300; and FIG. 14B, SN2310);

FIGS. 15A and 15B are graphs illustrating tumor growth (mm³) over time in xenographs treated with saline, irinotecan, and two representative tocopherol-modified therapeutic drug compounds of the invention (SN2300 and SN2310) in two different tumor models (FIG. 15A, NCI-H460; and FIG. 15B, HT-29); and

FIGS. 16A-16D show the chemical structures of representative diester-linked tocopherol-modified compounds of the invention.

### DETAILED DESCRIPTION OF THE INVENTION

In one aspect, the present invention provides therapeutic drug compounds that have been modified to increase their lipophilicity. The compounds of the invention are modified therapeutic drugs. The compounds of the invention include a therapeutic drug moiety and a lipophilic moiety.

In some embodiments, the compounds of the invention include more than one therapeutic drug moiety. In some embodiments, the compounds of the invention include more than one lipophilic moiety. In other embodiments, the compounds of the invention include more than one therapeutic drug moiety and more than one lipophilic moiety.

In some embodiments, the therapeutic drug moiety is covalently coupled to the lipophilic moiety through a linker moiety. In other embodiments, the therapeutic drug moiety is directly covalently coupled to the lipophilic moiety without a linker moiety.

In one embodiment, the lipophilic moiety is a tocopherol moiety, and the compound is a tocopherol-modified therapeutic drug compound. The tocopherol-modified therapeutic drug compound (or “tocopherolated” therapeutic drug compound) has one or more tocopherol moieties covalently coupled to a therapeutic drug moiety or a tocopherol moiety covalently coupled to one or more therapeutic drug moieties. As noted above, the tocopherol moiety is covalently coupled to the therapeutic drug moiety either directly or through a linker moiety.

In one embodiment, the tocopherol-modified therapeutic drug compounds of the invention can be represented by the general formula (1):
(T-L)ₙ(T)ₘD (1)
wherein T is a tocopherol moiety (i.e., a representative lipophilic moiety); L is a linker moiety; n is 0, 1, 2, or 3; m is 0, 1, 2, or 3; n+m is 1, 2, or 3; and D is a therapeutic drug moiety. In this embodiment, the compound includes n T-L moieties (i.e., tocopherol-linker moieties) and m tocopherol moieties, with the proviso that n+m is 1, 2, or 3. Each T-L moiety includes a tocopherol moiety covalently coupled to a linker moiety. Each of the n T-L moieties is covalently coupled to the therapeutic drug moiety through the T-L moiety's linker moiety. In this embodiment, each of the m tocopherol moieties is directly covalently coupled to the therapeutic drug moiety without a linker moiety.

Representative compounds having formula (1) include those in which n is 0 and m is 1, 2, or 3. These compounds have the general formula:
TₘD (2)
In this embodiment, one, two, or three tocopherol moieties are directly covalently coupled to the therapeutic drug moiety.

Representative compounds having formula (1) include those in which m is 0 and n is 1, 2, or 3. These compounds have the general formula:
(T-L)ₙD (3)
In this embodiment, one, two, or three T-L moieties are covalently coupled to the therapeutic drug moiety through the T-L moiety's linker moiety.

Representative compounds having formula (1) include those in which m is 1 or 2, and n is 1 or 2. These compounds have the general formulae:
(T-L)(T)D (4)
(T-L)(T)₂D (5)
(T-L)₂(T)D (6)
In these embodiments, the compounds of the invention have tocopherol moieties that are directly covalently coupled to the therapeutic drug moiety without a linker and tocopherol moieties that are covalently coupled to the therapeutic drug moiety through a linker (i.e., the T-L moieties).

The compounds of the invention described above include one therapeutic drug moiety and one or more lipophilic moieties (e.g., tocopherol moieties). In other embodiments, the compounds of the invention include more than one therapeutic drug moiety. In one embodiment, the compounds include two therapeutic drug moieties. In another embodiment, the compounds include three therapeutic drug moieties. For compounds that include more than one therapeutic drug moiety, the therapeutic drug moieties a can be the same or different.

For compounds that include more than one therapeutic drug moiety, the therapeutic drug moieties can be incorporated into the compound in any suitable way. In some embodiments, the therapeutic drug moieties can be directly covalently coupled (e.g., the compound includes a -D-D- or -D-D moiety). In other embodiments, the therapeutic drug moieties are separated in the compound by a linker moiety (e.g., the compound includes a -D-L-D- or -D-L-D moiety), a lipophilic moiety (e.g., the compound includes a -D-T-D- or -D-T-D moiety), or a lipophilic-linker moiety (e.g., the compound includes a -D-T-L-D-, -D-T-L-D, or -D-L-T-D moiety; or a -D-L-T-L-D- or -D-L-T-L-D moiety).

Representative compounds including two or three therapeutic drug moieties have the general formula:
(T-L)(D)ₚ (7)
wherein p is 2 or 3. In this embodiment, the two or three therapeutic drug moieties are covalently coupled to the linker moiety. In this instance, the linker includes multiple sites for the attachment of the therapeutic drug compound (or modified therapeutic drug compound). As is clear from formula (7), the linker moiety is also covalently coupled to the lipophilic moiety (e.g., tocopherol moiety). As noted above, compounds of the invention including more than one therapeutic drug moiety can have formulae other than shown above in formula (7). For example, such a compound can include more than one (e.g., two or three) lipophilic (e.g., tocopherol) moieties.

The compounds of the invention include one or more lipophilic moieties and one or more therapeutic drug moieties that are either directly covalently linked or covalently linked through linker moieties.

As used herein, the term “lipophilic moiety” refers to a chemical moiety having lipophilic or hydrophobic characteristics and that increases the solubility of a therapeutic drug compound in a lipophilic solvent or environment when covalently coupled to the therapeutic drug compound to provide a compound of the invention. A description of representative lipophilic moieties useful in making the compounds of the invention is provided below.

As used herein, the term “therapeutic drug moiety” refers to a chemical moiety derived from a therapeutic drug compound. A description of representative therapeutic drug compounds useful in making the compounds of the invention is provided below.

As used herein, the term “linker moiety” refers to an atom or a group of atoms that covalently link, for example, a lipophilic moiety to a therapeutic drug moiety. A description of representative linkers useful in making the compounds of the invention is provided below.

Lipophilic Modification of Therapeutic Drug Compounds. A therapeutic drug compound may have one or more suitable functional groups, or may be modified to include one or more suitable functional groups for covalent coupling to a lipophilic moiety. Suitable functional groups include, for example, the following groups: hydroxyl group (—OH), amino group (primary amino group, —NH₂, or secondary amino group, —NHR₁, where R₁ is independently selected from H, C₁₋₆ n-alkyl, C₃₋₁₂ branched alkyl, substituted or unsubstituted C₃₋₆ cycloalkyl, substituted or unsubstituted aryl, or aralkyl), thiol group (—SH), carboxyl group (—COOH), aldehyde group (—CHO), isocynato group (—N═C═O), sulfonic acid group (—SO₃H), sulfuric acid group (—OSO₃H), phosphoric acid group (—OPO₃H), phosphonic acid group (—PO₃H₂), allylic halide group, benzylic halide group, substituted benzylic halide group, and oxiranyl group (—CH(O)CH₂).

A therapeutic drug compound may be directly coupled to a lipophilic moiety (e.g., a tocopherol moiety) through an ester group (—C(═O)O—), carbamate group (—OC(═O)NH—), sulfonate group (—SO₃—), sulfate group (—OSO₃—), phosphate group (—OPO₃R₁—, where R₁ is independently selected from H, C₁₋₆ n-alkyl, C₃₋₁₂ branched alkyl, substituted or unsubstituted C₃₋₆ cycloalkyl, substituted or unsubstituted aryl, or aralkyl), phosphonate group (—PO₃R₁—, where R₁ is independently selected from H, C₁₋₆ n-alkyl, C₃₋₁₂ branched alkyl, substituted or unsubstituted C₃₋₆ cycloalkyl, substituted or unsubstituted aryl, or aralkyl), or ether group (—O—).

A tocopherol compound, a representative lipophilic compound suitable for making the compounds of the invention, includes a hydroxyl group (—OH). After modification, a tocopherol compound may be covalently coupled to a linker compound that includes one or more reactive functional groups. Suitable reactive functional groups include the following groups: hydroxyl group (—OH), amino group (primary amino group, —NH₂, or secondary amino group, —NHR₁, where R₁ is independently selected from H, C₁₋₆ n-alkyl, C₃₋₁₂ branched alkyl, substituted or unsubstituted C₃₋₆ cycloalkyl, substituted or unsubstituted aryl, or aralkyl.), thiol group (—SH), carboxyl group (—C(═O)OH), aldehyde group (—CHO), isocynato group (—N═C═O), sulfonic acid group (—SO₃H), sulfuric acid group (—OSO₃H), phosphoric acid group (—OPO₃H), phosphonic acid group (—PO₃H₂), allylic halide group, benzylic halide group, substituted benzylic halide group, and oxiranyl group (—CH(O)CH₂).

Linker Moieties. As noted above, in some embodiments, the compounds of the invention include a lipophilic moiety (e.g., tocopherol moiety) covalently coupled to a therapeutic drug moiety by a linker moiety. In addition to the embodiments described above, the tocopherol-modified therapeutic drug compounds of the invention can be represented by the general formula (8):
T-A-R-A′-D (8)
where T is a tocopherol moiety (i.e., a representative lipophilic moiety), D is a therapeutic drug moiety, and A-R-A′ is a linker moiety. It will be appreciated that for formulae (1) and (3)-(7) above, each of which includes linker moiety L, the linker moiety L in those compounds can be linker moiety A-R-A′.

In formula 8, groups A and A′ are independently selected from O, S, SO, SO₂, NR₁, carboxylate group (—C(═O)O—), amide group (—C(═O)NR₁—), anhydride group (—C(═O)OC(═O)—), carbamate group (—OC(═O)NH—), carbonyldioxy group (—OC(═O)O—), ureylene group (—NR₁C(═O)NR₂—), phosphate group (—OP(═O)(OR₁)O—), phosphamide group (—OP(═O)(NR₁)O—), phosphonate group (—OP(OR₁)O—), phosphonamide group (—OP(═O)NR₁—), sulfate group (—OSO₂₀—), sulfamide group (—SO₂NR₁—), sulfonate group (—SO₃—), sulfonamide group (—SO₂NR₁—), and the dicarbonyl group, —C(═O)R₃C(═O)—, where R₃ is absent or a divalent alkyl (e.g., —(CH₂)ₙ—, n=1-12), substituted alkyl, branched alkyl, cycloalkyl, substituted cycloalkyl, aryl, substituted aryl. For the above groups, R is a divalent group selected from the following groups: alkyl; substituted alkyl, branched alkyl; cycloalkyl; substituted cycloalkyl; heteroalkyl; substituted heteroalkyl; aryl; substituted aryl; aralkyl; substituted aralkyl; amino acid; peptide; polypeptide; protein; mono-, di- or polysaccharide; oligomer of ethylene glycol, poly(ethylene glycol); poly(alkylene oxide) polymers, such as poly(ethylene oxide) and poly(propylene oxide); and poly(ethylene oxide)-poly(propylene oxide) copolymer. For the above groups, R₁ and R₂ are independently selected from Na⁺, K⁺, H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl.

As used herein, the term “aryl” refers to monocyclic and polycyclic aromatic compounds having from 6 to 14 carbon or hetero atoms, and includes carbocyclic aryl groups and heterocyclic aryl groups. Representative aryl groups include phenyl, naphthyl, pyridinyl, pyrimidinyl, thiazolyl, indolyl, imidazolyl, furanyl, and the like. The term “aralkyl” refers to an alkyl group that is substituted with an aryl group.

The following compounds (compounds 9 to 32) are representative examples of compounds having formula 8.

A linker moiety (L) and therapeutic drug moiety (D) may be covalently coupled through an ester group. In one embodiment, the therapeutic drug moiety includes a hydroxyl group that is coupled with a carboxyl group of the linker moiety. The linker moiety may be coupled to a tocopherol moiety through an ether group (compound 9), ester group (compound 10), amine group (compound II), or amide group (compound 12).

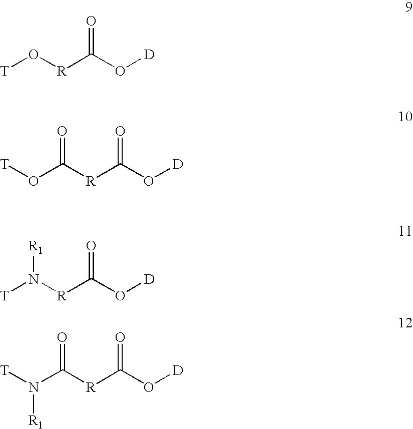

In another embodiment, the therapeutic drug moiety (D) includes a carboxyl group that is coupled with a hydroxyl group of the linker moiety (L). The linker moiety may be coupled to a tocopherol moiety through an ether group (compound 13), ester group (compound 14), amine group (compound 15), or amide group (compound 16).

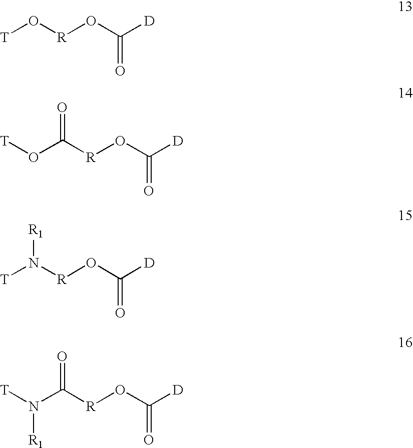

In the above compounds, divalent group R is selected from alkyl; substituted alkyl; branched alkyl; cycloalkyl; substituted cycloalkyl; heteroalkyl; substituted heteroalkyl; aryl; substituted aryl; aralkyl; substituted aralkyl; amino acid; peptide; polypeptide; protein; mono-, di- or polysaccharide; oligomer of ethylene glycol, poly(ethylene glycol); poly(alkylene oxide) polymers, such as poly(ethylene oxide) and poly(propylene oxide); and poly(ethylene oxide)-poly(propylene oxide) copolymer. In the above compounds, R₁ is selected from H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl.

While the preferred embodiment of the invention has been illustrated and described, it will be appreciated that various changes can be made therein without departing from the spirit and scope of the invention. A linker moiety (L) and therapeutic drug moiety (D) may be covalently coupled through an amide group. In one embodiment, the therapeutic drug moiety includes an amine group that is coupled a carboxyl group of the linker moiety. The linker moiety may be coupled to a tocopherol moiety through an ether group (compound 17), ester group (compound 18), amine group (compound 19), or amide group (compound 20).

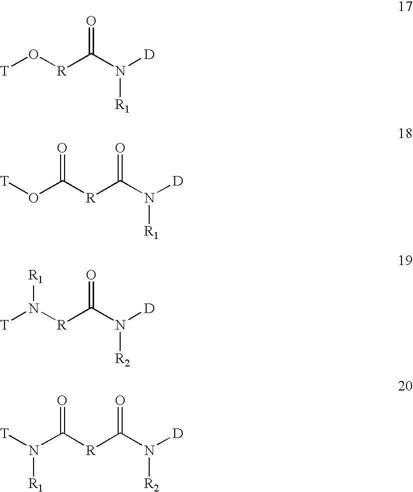

In the above compounds, divalent group R is selected from alkyl; substituted alkyl; branched alkyl; cycloalkyl; substituted cycloalkyl; heteroalkyl; substituted heteroalkyl; aryl; substituted aryl; aralkyl; substituted aralkyl; amino acid; peptide; polypeptide; protein; mono-, di- or polysaccharide; oligomer of ethylene glycol, poly(ethylene glycol); poly(alkylene oxide) polymers, such as poly(ethylene oxide) and poly(propylene oxide); and poly(ethylene oxide)-poly(propylene oxide) copolymer. In the above compounds, R₁ and R₂ are independently selected from H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl.

A linker moiety (L) and a therapeutic drug moiety (D) may be covalently coupled through an ether group (compound 21) or amine group (compound 22). In one embodiment, the therapeutic drug moiety includes a hydroxy group, and in another embodiment, the therapeutic drug moiety includes an amine group. The linker moiety may be coupled to a tocopherol moiety through an ether group.

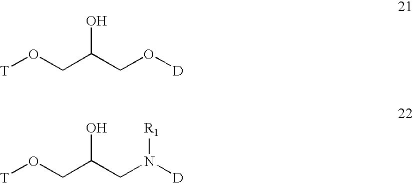

In the above compounds, R₁ is selected from H, C₁₋₆ n-alkyl, C₃₋₁₂ branched alkyl, substituted or unsubstituted C₃₋₆ cycloalkyl, substituted or unsubstituted aryl, or aralkyl.

A tocopherol moiety (T) and a therapeutic drug moiety (D) may be covalently coupled through a carbonyldioxy group (—OC(═O)O—) (compound 23). In this case, the linker moiety is the carbonyldioxy group and the therapeutic drug moiety includes a hydroxyl group.

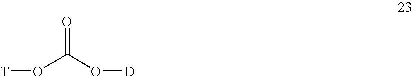

A tocopherol moiety (T) and a therapeutic drug moiety (D) may be covalently coupled through an anhydride group (—C(═O)OC(═O)—). In one embodiment, the therapeutic drug moiety includes a carboxyl group that is coupled with a carboxyl group of the linker moiety. The linker moiety may be coupled to a tocopherol moiety through an ether group (compound 24), ester group (compound 25), amine group (compound 26), or amide group (compound 27).

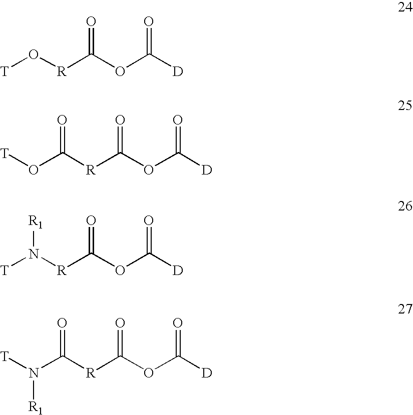

In the above compounds, divalent group R is selected from alkyl; substituted alkyl; branched alkyl; cycloalkyl; substituted cycloalkyl; heteroalkyl; substituted heteroalkyl; aryl; substituted aryl; aralkyl; substituted aralkyl; amino acid; peptide; polypeptide; protein; mono-, di- or polysaccharide; oligomer of ethylene glycol, poly(ethylene glycol); poly(alkylene oxide) polymers, such as poly(ethylene oxide) and poly(propylene oxide); and poly(ethylene oxide)-poly(propylene oxide) copolymer. In the above compounds, R₁ is selected from H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl.

A tocopherol moiety (T) and therapeutic drug moiety (D) may be covalently coupled through a phosphate, phosphoramide, or thiophosphate group (compound 28).

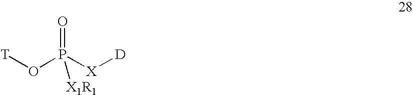

In the above compounds, X is O, NR₂, or S; X₁ is O, NR₃, or S; and R₁ is selected from Na⁺, K⁺, H, C₁₋₆ n-alkyl, C₃₋₁₂ branched alkyl, substituted or unsubstituted C₃₋₆ cycloalkyl, substituted or unsubstituted aryl, or aralkyl; and R₂ and R₃ are independently selected from C₁₋₆ n-alkyl, C₃₋₁₂ branched alkyl, substituted or unsubstituted C₃₋₆ cycloalkyl, substituted or unsubstituted aryl, or aralkyl.

A tocopherol moiety (T) and a therapeutic drug moiety (D) may be covalently coupled through a sulfate, thiosulfate, or sulfonamide group (compound 29).

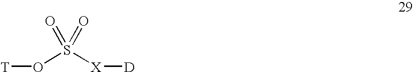

In the above compounds, X is O, NR₁, or S; and R₁ is selected from H, C₁₋₆ n-alkyl, C₃₋₁₂ branched alkyl, substituted or unsubstituted C₃₋₆ cycloalkyl, substituted or unsubstituted aryl, or aralkyl.

A tocopherol moiety (T) and therapeutic drug moiety (D) may be covalently coupled through a ureylene group (—NHC(═O)NH—) (compound 30).

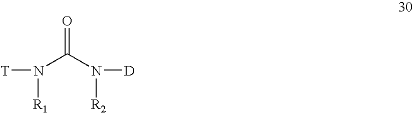

In the above compounds, R₁ and R₂ are independently selected from H, C₁₋₆ n-alkyl, C₃₋₁₂ branched alkyl, substituted or unsubstituted C₃₋₆ cycloalkyl, substituted or unsubstituted aryl, or aralkyl.

A tocopherol moiety (T) and a therapeutic drug moiety (D) may be covalently coupled through a carbamate group (—NR₁C(═O)O— or —OC(═O)NR₂—, compounds 31 and 32, respectively).

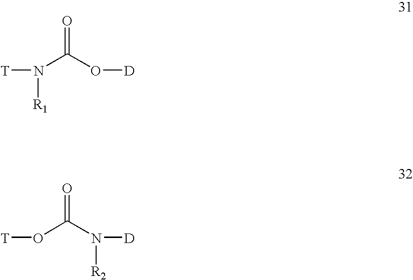

In the above compounds, R₁ and R₂ are independently selected from H, C₁₋₆ n-alkyl, C₃₋₁₂ branched alkyl, substituted or unsubstituted C₃₋₆ cycloalkyl, substituted or unsubstituted aryl, or aralkyl.

Lipophilic Moiety. The compounds of the invention include one or more lipophilic moieties. The lipophilic moiety or moieties increases the solubility of the compound in a lipophilic solvent or environment. In one embodiment, the lipophilic moiety is a tocopherol moiety.

As used herein, the term “tocopherol moiety” refers to a chemical moiety that is derived from a family of natural or synthetic compounds, also known by their generic names, tocol or vitamin E. In addition to tocopherol compounds, tocotrienol compounds are included in this family. These compounds include a chroman head having a phenolic alcohol (C-6) and a phytyl tail (C-2). These compounds have the following general formula:

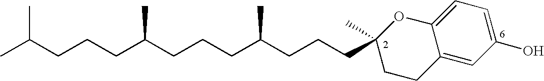

Tocopherols constitute a series of related benzopyranols (or methyl tocols) in which the C-2 phytyl (sixteen carbon) side chain is saturated. Representative tocopherols include α-tocopherol, (d-form, dl-form, l-form), β-tocopherol (d-form, dl-form, l-form), 7-tocopherol (d-form, dl-form, l-form), and δ-tocopherol (d-from, dl-form, l-form). Among tocopherols, α-tocopherol is the most abundant. Tocotrienols are similar in structure to tocopherols except that the trienols have three double bonds in the C-2 phytyl side chain.

Tocopherol and tocotrienol compounds useful in making the compounds of the invention include those shown below.

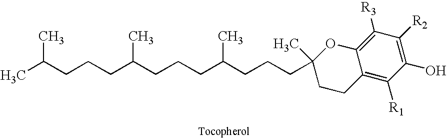

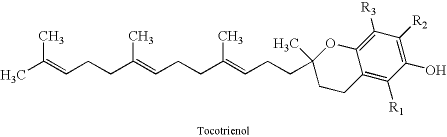

| Compound | R₁ | R₂ | R₃ |
|---|---|---|---|
| alpha (α) | CH₃ | CH₃ | CH₃ |
| beta (β) | CH₃ | H | CH₃ |
| gamma (γ) | H | CH₃ | CH₃ |
| delta (δ) | H | H | CH₃ |

As used herein, the term “tocopherol” refers to any member of the tocopherol family noted above.

Therapeutic Drug Moiety. The compounds of the invention include one or more therapeutic drug moieties. Virtually any therapeutic drug compound having a suitable functional group, or that can be modified to include a suitable functional group, can be covalently coupled to a lipophilic compound to provide a compound of the invention. Representative functional groups include, for example, hydroxyl groups (—OH), amino groups (primary amino groups, —NH₂, and secondary amino groups, —NHR), thiol groups (—SH), carboxyl groups (—COOH), aldehyde groups (—CHO), isocynato groups (—N═C═O), sulfonic acid groups (—SO₃H), sulfuric acid groups (—OSO₃H), phosphoric acid groups (—OPO₃H), phosphonate groups (—PO₃OR₁R₂, and R₁ and R₂ are independently selected from H, C₁₋₆ n-alkyl, C₃₋₁₂ branched alkyl, substituted or unsubstituted C₃₋₆ cycloalkyl, substituted or unsubstituted aryl, or aralkyl.), allylic halide group, benzylic halide group, substituted benzylic halide group, and oxiranyl group (—CH(O)CH₂).

Therapeutic drug compounds useful in making the compounds of the invention need not be substantially water insoluble, although tocopherol modification according to the present invention is especially well suited for formulating and delivering such water-insoluble compounds.

In one embodiment, the therapeutic drug moiety is derived from a therapeutic compound that is substantially insoluble in water. In another embodiment, the therapeutic drug moiety is derived from a therapeutic compound that is substantially insoluble in organic solvents. In another embodiment, the therapeutic drug moiety is derived from a therapeutic compound that is substantially insoluble in water and substantially insoluble in organic solvents. In one embodiment, the therapeutic drug compound has a solubility in water at room temperature less than about 1000 μg/mL. In one embodiment, the therapeutic drug compound has a solubility in water at room temperature less than about 500 μg/mL. In one embodiment, the therapeutic drug compound has a solubility in water at room temperature less than about 100 μg/mL. In one embodiment, the therapeutic drug compound has a solubility in water at room temperature less than about 25 μg/mL.

Representative therapeutic compound drugs useful in making the compounds of the invention include anticancer compounds (e.g., paclitaxel and its derivatives including docetaxel, camptothecin and its derivatives including 7-ethyl-10-hydroxycamptothecin (SN38) and 10-hydroxycamptothecin, and doxorubicin and its derivatives), antifungal compounds (e.g., flucanazole), antibacterial compounds (e.g., penicillin G, penicillin V), anti-hypertensive compounds (e.g., hydralazine, candesartan, and carvediol), anti-inflammatory compounds (e.g., isoxicam), antidiabetic compounds (e.g., metformin), antiviral compounds (e.g., lamivudine), antidepressant compounds (e.g., fluoxetine), antihistaminic compounds (e.g., hydroxyzine), anti-arrhythmic compounds (e.g., procainamide hydrochloride), anti-hyperlipoproteinemic compounds (e.g., probucol), and compounds for reproductive health (e.g., danazol), and treating Parkinson's disease (e.g., lazabemide), and immunosuppressive (e.g., azathioprine and cyclosporine) and respiratory (e.g., bosentan) diseases and conditions. Other therapeutically useful biological materials that can be modified according to the invention, include biologically active proteins, enzymes, and peptides.

In one embodiment, the therapeutic drug moiety is derived from an anticancer compound. Representative anticancer therapeutic compounds include taxanes. Taxanes include any anti-mitotic taxane, taxane derivative or analog. As used herein, the term “taxane” refers to taxanes, taxines, and taxoids, as well as derivatives or analogs thereof.

Paclitaxel and its derivatives and analogs are members of the taxane family. Paclitaxel derivatives include, for example benzoate derivatives of paclitaxel such as 2-debenzoyl-2-aroyl and C-2-acetoxy-C-4-benzoate paclitaxel, 7-deoxytaxol, C-4 aziridine paclitaxel, as well as various paclitaxel conjugates with natural and synthetic polymers, particularly with fatty acids, phospholipids, and glycerides and 1,2-diacyloxypropane-3-amine. Other paclitaxel derivatives include docetaxel; spicatin; taxane-2,13-dione, 5β,9β,10β-trihydroxy-, cyclic 9,10-acetal with acetone, acetate; taxane-2,13-dione, 5β,9β, 10β-trihydroxy-, cyclic 9,10-acetal with acetone; taxane-2β,5β,9β,10β-tetrol, cyclic 9,10-acetal with acetone; taxane; cephalomannine-7-xyloside; 7-epi-10-deacetylcephalomannine; 10-deacetylcephalomannine; cephalomannine; taxol B; 13-(2′,3′-dihydroxy-3′phenylpropionyl)baccatin III; yunnanxol; 7-(4-azidobenzoyl)baccatin III; N-debenzoyltaxol A; O-acetylbaccatin IV; 7-(triethylsilyl)baccatin III; 7,10-di-O—[(2,2,2,-trichloroethoxy)carbonyl]baccatin III; baccatin III 13-O-acetate; baccatin diacetate; baccatin; baccatin VII; baccatin VI; baccatin IV; 7-epi-baccatin III; baccatin V; baccatin I; baccatin III; baccatin A; 10-deactyl-7-epitaxol; epitaxol; 10-deacetyltaxol C; 7-xylosyl-10-deacetyltaxol; 10-deacetyltaxol-7-xyloside; 7-epi-10-deacetyltaxol; 10-deactyltaxol; or 10-deactyltaxol B.

Other anticancer compounds useful in making the compounds of the invention include camptothecin and its derivatives including 7-ethyl-10-hydroxycamptothecin (SN38) and 10-hydroxycamptothecin, and doxorubicin and its derivatives.

In certain embodiments, the therapeutic drug moiety is derived from paclitaxel, docetaxel, camptothecin, or their derivatives.

For compounds of the invention having formula (2) with m=1, formula (3) with n=1, and formula 8, certain compounds are excluded and are not within the scope of the invention. When the linker moiety is 2-hydroxypropylene (—CH₂CH(OH)CH₂—), the therapeutic drug moiety is not an α-amino acid (e.g., glycine, alanine, proline, cysteine, aminobutyric acid, aspartic acid, glutamic acid), an co-amino acid (e.g., β-alanine, γ-aminobutyric acid, ε-aminocaproic acid, 2-aminoethanesulfonic acid (taurine)), or a peptide containing a cysteine residue bonding through its N-terminus or thiol group (e.g., -glutathione). When the linker moiety is a succinate, the therapeutic drug moiety is not an S-linked amino or amino acid compound coupled to one of the aliphatic succinate carbons. When the linker moiety is succinate, the therapeutic drug moiety is not ferulic acid or an ester thereof.

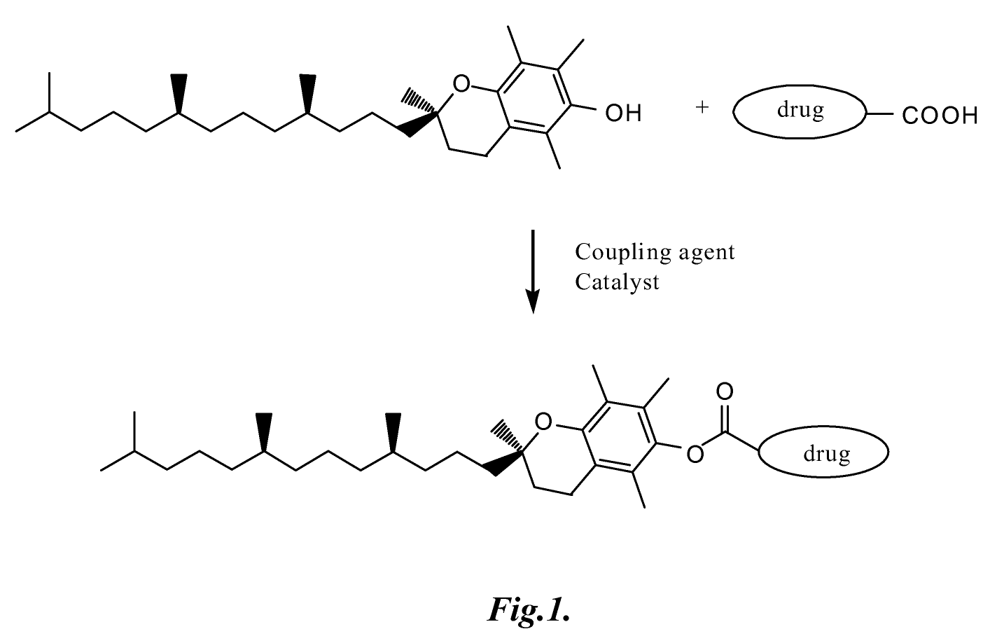

In another aspect, methods for making the compounds of the invention are provided. There are many ways to covalently couple a lipophilic compound (e.g., a tocopherol compound) to a therapeutic drug compound to form a compound of the invention. In one embodiment, a representative tocopherol, d-α-tocopherol, includes a hydroxyl group that may be directly coupled with a carboxyl group of a therapeutic drug to form a tocopherol-modified therapeutic drug compound. The preparation of a representative tocopherol-modified therapeutic compound of the invention from a carboxylic acid-containing therapeutic drug compound is illustrated in FIG. 1.

In another embodiment, a tocopherol may be functionalized at the hydroxyl group with a reagent to attach an active group such as phosphoric chloride (—P(O)OR₁Cl), phosphonic chloride (—P(O)R₁Cl), sulfonic chloride (—SO₂Cl), or carbonyl chloride (—COCl). The resulting acid chloride can then be reacted with an appropriately functionalized therapeutic drug compound to provide a tocopherol-modified therapeutic drug compound.

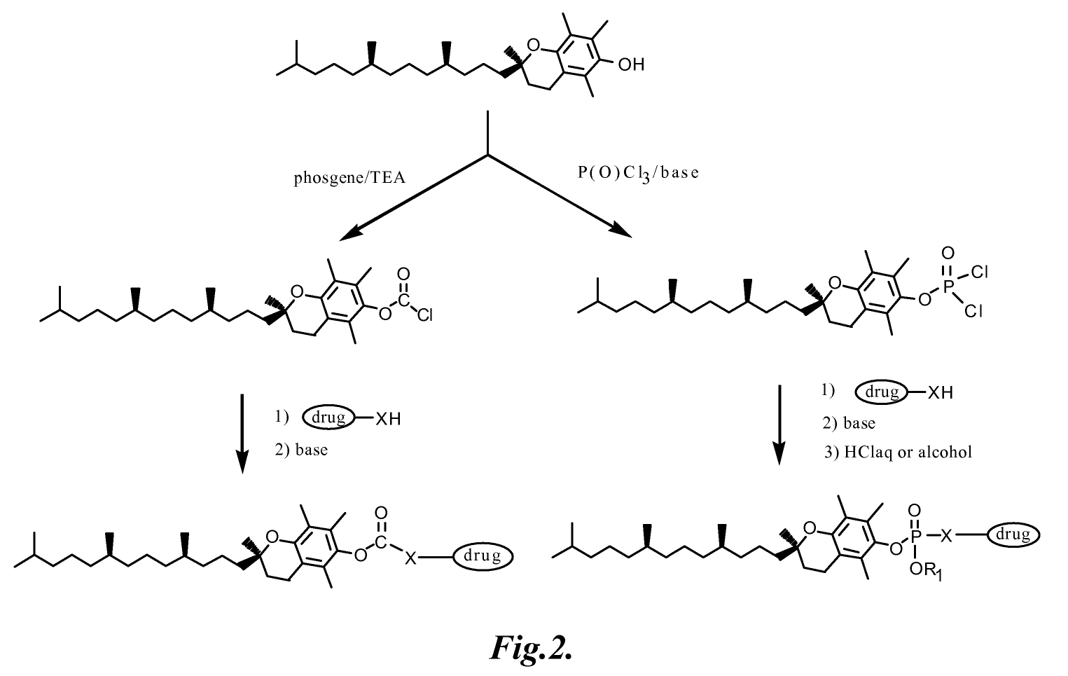

In FIG. 2, X is O, S, or NH; and R₁ is independently selected from H, C₁₋₆ n-alkyl, C₃₋₁₂ branched alkyl, substituted or unsubstituted C₃₋₆ cycloalkyl, substituted or unsubstituted aryl, and substituted or unsubstituted aralkyl.

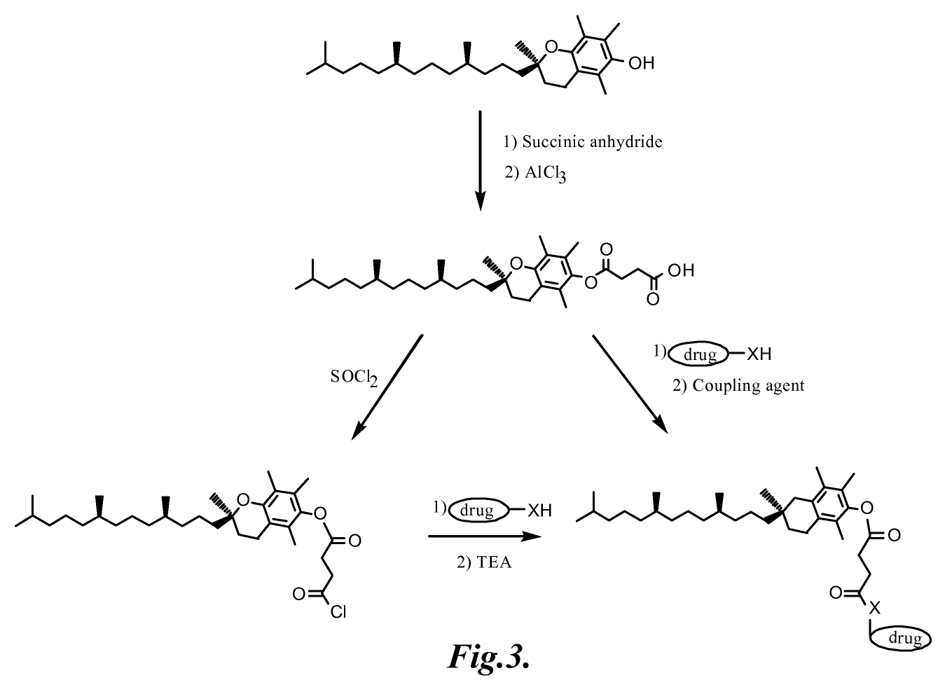

In another embodiment, a tocopherol may be functionalized at the hydroxyl group with a dicarboxylic acid, ester, or anhydride reagent. Suitable reagents include succinic acid anhydride, 1,2-cyclohexanedicarboxylic anhydride, 2,3-dimethylsuccinic anhydride, 3,3-tetramethylene glutaric anhydride, glutaric anhydride, maleic acid anhydride, phthalic acid anhydride, terephthalic acid, or isophthalic acid to attach a carboxyl group (—COOH). The resulting carboxyl group may then be directly reacted with an appropriately functionalized therapeutic drug or the carboxyl group may be converted to a more reactive carbonyl chloride group (—COCl), and then the carbonyl chloride group may be coupled with the functional group of the therapeutic drug to form a tocopherol-modified therapeutic drug compound as illustrated in FIG. 3. In FIG. 3, X is O, S, NH, or C(═O)O.

In another embodiment, a linker can be coupled to the hydroxyl group of a tocopherol and then a therapeutic drug can be coupled to an accessible functional group on the linker. The functional group may be, for example, but not limited to, a carboxyl group (—COOH), a poly(ethylene oxide) group (—(CH₂CH₂O)ₙ—H), an aldehyde group (—CHO), an isocyanato group (—N═C═O), a phosphoric acid group (—OPO₃H₂), or phosphoric chloride group (—OPO₂R¹Cl, where R₁ is a substituted or unsubstituted alkyl or cycloalkyl, a substituted or unsubstituted aryl, or an aralkyl), a phosphonic chloride group (—PO₂R¹Cl, where R₁ is a substituted or unsubstituted alkyl or cycloalkyl, a substituted or unsubstituted aryl, or an aralkyl), a sulfuric acid group (—OSO₃H₂), a chlorosulfuric group (—SO₃Cl), or an oxiranyl group (—CH(O)CH₂).

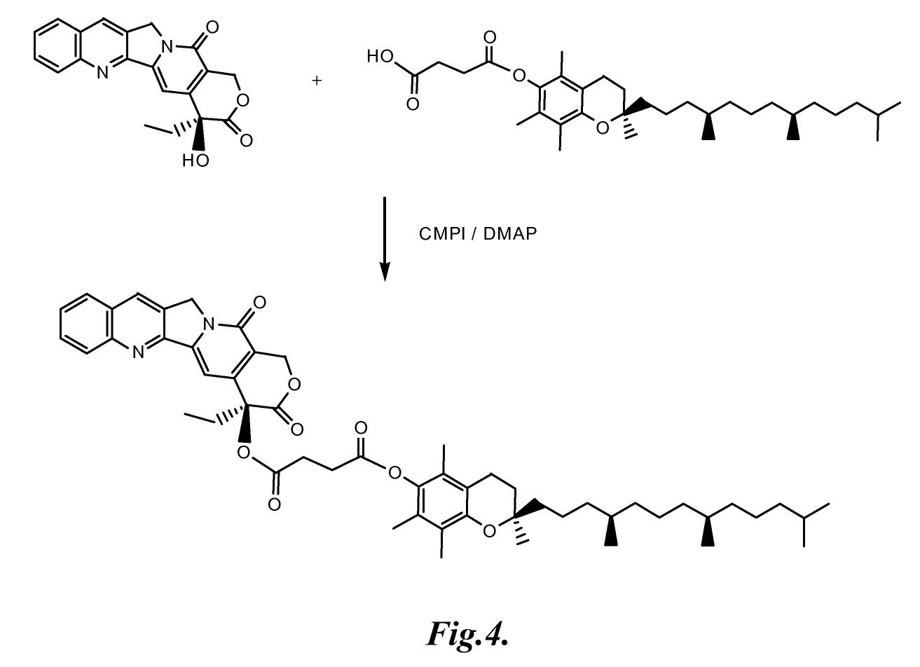

The syntheses of representative tocopherol-modified therapeutic drug compounds of the invention are illustrated in FIGS. 4-11 and described in Examples 1-13.

FIG. 4 illustrates the preparation of a tocopherol succinate camptothecin compound. Tocopherol succinic acid (vitamin E succinic acid) has a free carboxyl group that can couple with a hydroxyl group, amino group, thiol group, or carbonyl chloride group to provide a tocopherol-modified therapeutic drug having a succinate group as a linker. In FIG. 4, the carboxyl group of tocopherol acid succinate is coupled with the hydroxyl group of camptothecin. The preparation of tocopherol succinate camptothecin (Butanedioic acid, (2R)-3,4-dihydro-2,5,7,8-tetramethyl-2-[(4R,8R)-4,8,12-trimethyltridecyl]-2H-1-benzopyran-6-yl (4S)-4-ethyl-3,4,12,14-tetrahydro-3,14-dioxo-1H-pyrano[3′,4′:6,7]indolizino[1,2-b]quinolin-4-yl ester) is described in Example 1.

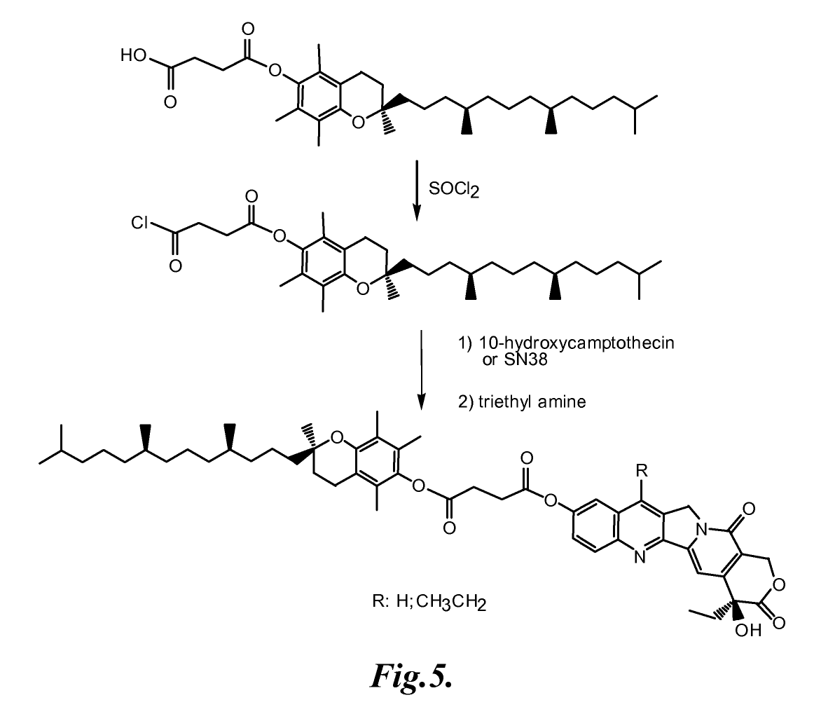

FIG. 5 illustrates the preparation of tocopherol succinate 10-hydroxycamptothecin and tocopherol succinate 7-ethyl-10-hydroxycamptothecin (SN38). Tocopherol succinic acid is converted to the corresponding acid chloride, and then reacted with 10-hydroxycamptothecin or 7-ethyl-10-hydroxycamptothecin (SN38). The preparations of tocopherol succinate 10-hydroxycamptothecin (Butanedioic acid, (2R)-3,4-dihydro-2,5,7,8-tetramethyl-2-[(4R,8R)-4,8,12-trimethyltridecyl]-2H-1-benzopyran-6-yl (4S)-4-ethyl-3,4,12,14-tetrahydro-4-hydroxy-3,14-dioxo-1H-pyrano[3′,4′:6,7]indolizino[1,2-b]quinolin-9-yl ester) and tocopherol succinate 7-ethyl-10-hydroxycamptothecin (Butanedioic acid, (4S)-4,11-diethyl-3,4,12,14-tetrahydro-4-hydroxy-3,14-dioxo-1H-pyrano[3′,4′:6,7]indolizino[1,2-b]quinolin-9-yl (2R)-3,4-dihydro-2,5,7,8-tetramethyl-2-[(4R,8R)-4,8,12-trimethyltridecyl]-2H-1-benzopyran-6-yl ester) are described in Examples 2 and 3, respectively.

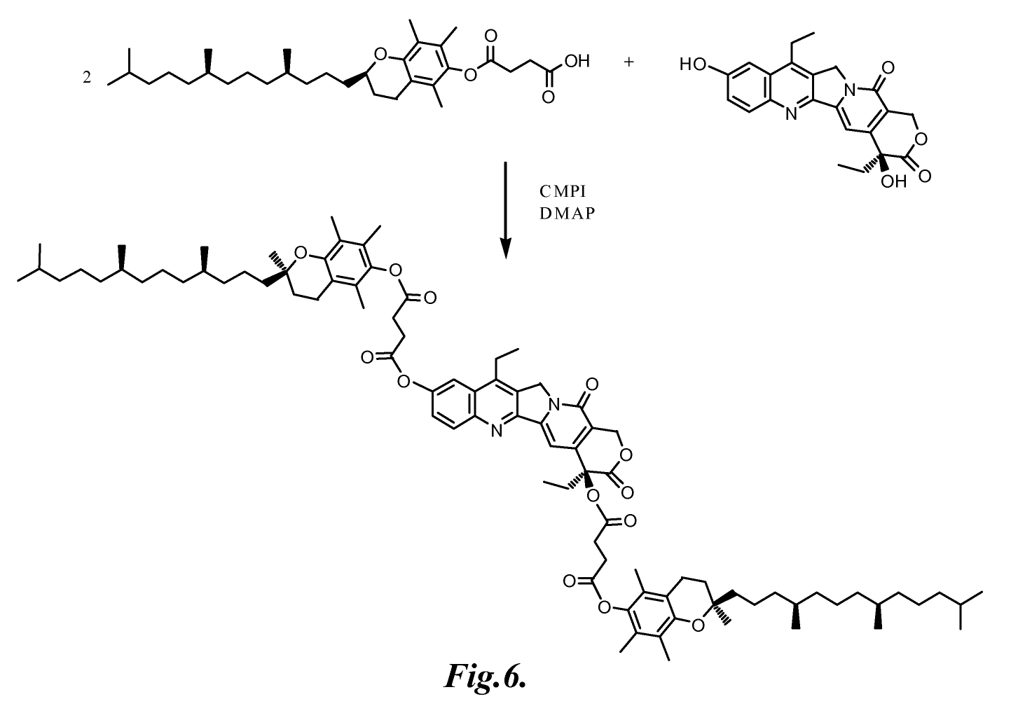

FIG. 6 illustrates the preparation of 10,20-di(tocopherol succinate) SN38, which contains one therapeutic drug (SN38) moiety, two tocopherol moieties, and two linker moieties (succinyl groups). The preparation of 10,20-di(tocopherol succinate) SN38 is described in Example 4.

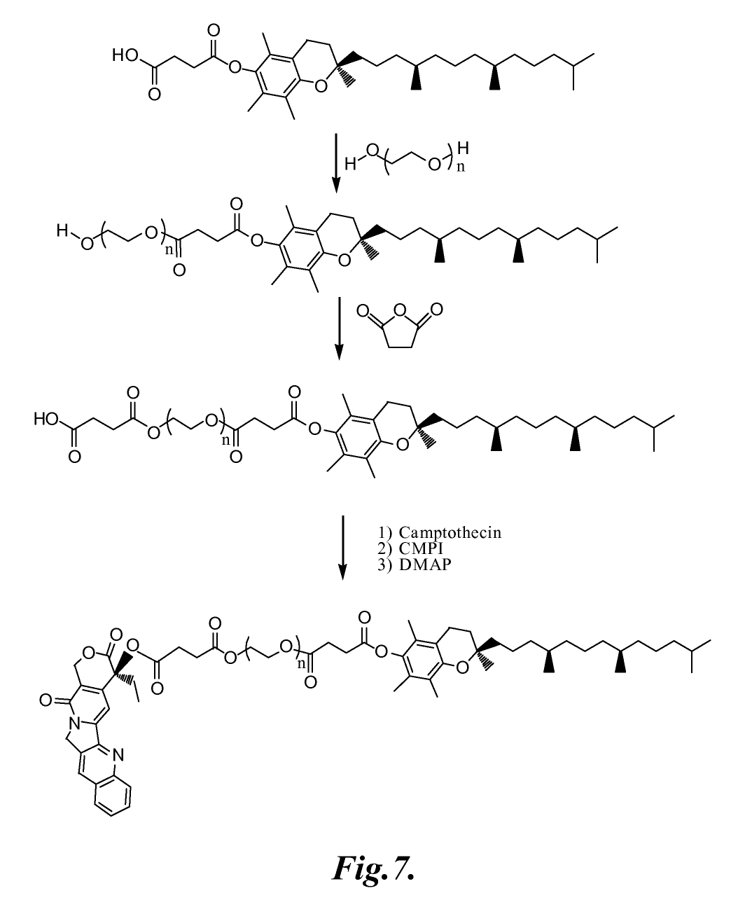

Suitable linker moieties can include an oligomer or polymer such as a peptide, polypeptide, protein, mono-, di- or polysaccharide, oligomer of ethylene glycol, poly(ethylene glycol), poly(alkylene oxide) such as poly(ethylene oxide) and poly(propylene oxide), or poly(ethylene oxide)-poly(propylene oxide) copolymer. FIG. 7 illustrates the preparation of a tocopherol-modified camptothecin containing a linker moiety that includes a poly(ethylene oxide) group. The preparation of the tocopherol succinate camptothecin having a linker moiety that includes a poly(ethylene oxide) group is described in Example 5.

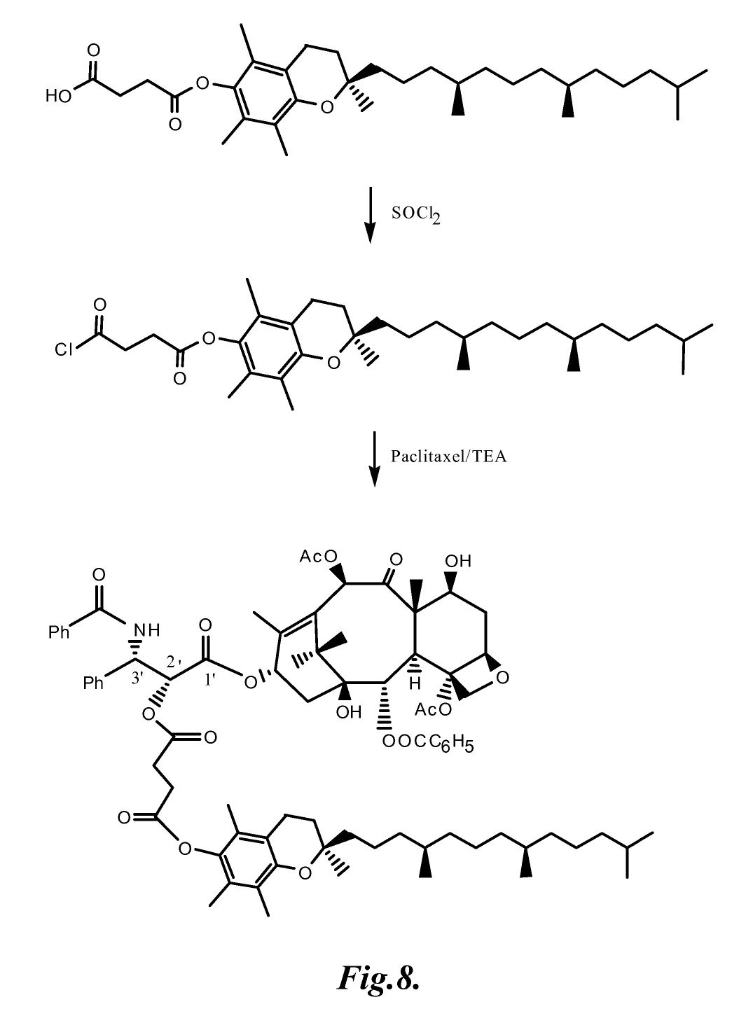

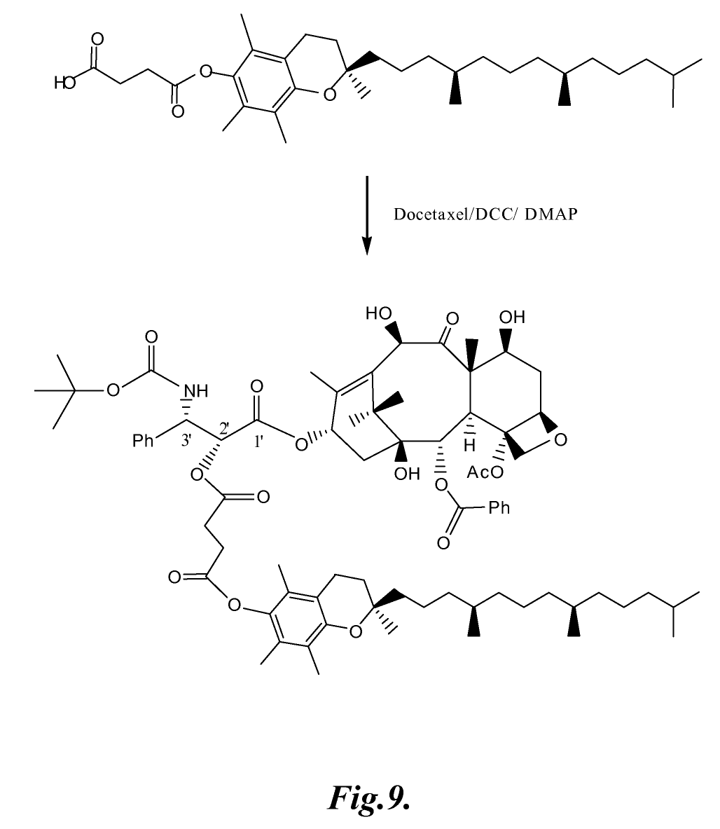

FIG. 8 illustrates the preparation of tocopherol succinate paclitaxel. In the preparation, tocopherol succinic acid is converted to the corresponding acid chloride and then reacted with paclitaxel. The preparation of tocopherol succinate paclitaxel is described in Example 6. FIG. 9 illustrates the preparation of tocopherol succinate docetaxel. The preparation of tocopherol succinate docetaxel is described in Example 7.

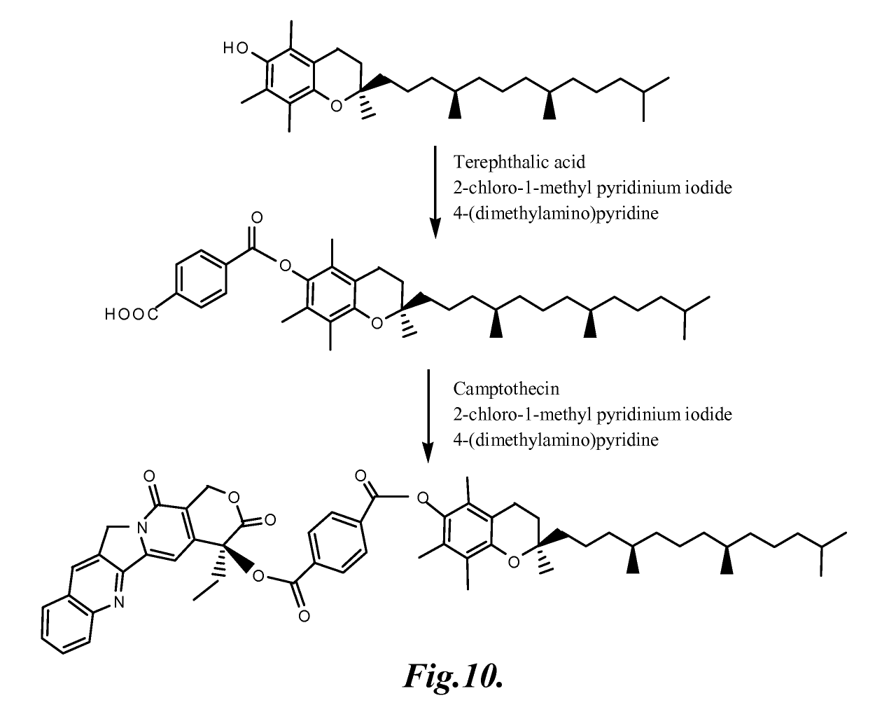

FIG. 10 illustrates the preparation of tocopherol terephthalate camptothecin. In the preparation, tocopherol is first conjugated with terephthalate to form tocopherol terephthalate (Example 9) and is then coupled with camptothecin to form tocopherol terephthalate camptothecin. The preparation of tocopherol terephthalate camptothecin is described in Example 10.

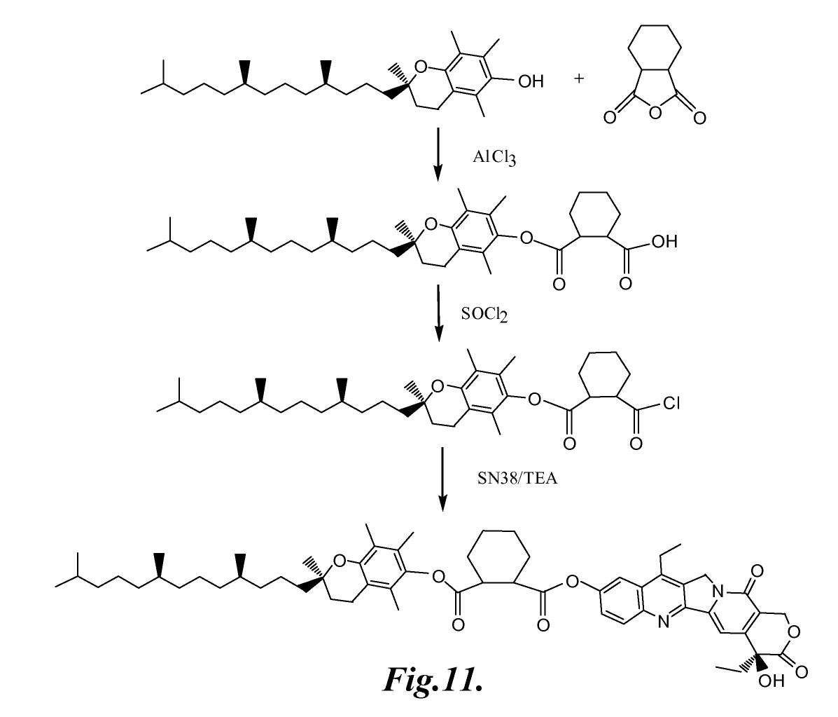

FIG. 11 illustrates the preparation of tocopherol cyclohexane-1,2-dicarboxylate SN38. The preparation of tocopherol cyclohexane-1,2-dicarboxylate SN38 is described in Example 11.

The preparations of tocopherol succinate doxorubicin and tocopherol succinate hydroxyzine are described in Examples 12 and 13, respectively.

In one aspect, the compounds of the invention are diester-linked compounds. As used herein the term “diester-linked compound” refers to a tocopherol-modified therapeutic drug compound of the invention in which the tocopherol moiety is covalently coupled to the therapeutic drug moiety through a diester (—O—C(═O)—R—C(═O)—) moiety.

In one embodiment, compounds of the invention have formula (I):
T-O—C(═O)—R—C(═O)—O-D (I)

wherein

T is a tocopherol moiety derived from a tocopherol selected from the group consisting of:

(a) α-tocopherol,

(b) β-tocopherol,

(c) γ-tocopherol,

(d) δ-tocopherol,

(e) α-tocotrienol,

(f) β-tocotrienol,

(g) γ-tocotrienol, and

(h) δ-tocotrienol;

D is a therapeutic drug moiety derived from a therapeutic drug selected from the group consisting of:

(a) paclitaxel and its derivatives,

(b) docetaxel and its derivatives, and

(c) camptothecin and its derivatives; and

R is a divalent moiety selected from the group consisting of:

(a) substituted and unsubstituted alkyl,

(b) substituted and unsubstituted cycloalkyl, and

(c) substituted and unsubstituted aryl.

In one embodiment, the tocopherol moiety is selected from an α-tocopherol moiety and a δ-tocopherol moiety. In one embodiment, the tocopherol moiety is a dl-α-tocopherol moiety.

In one embodiment, the therapeutic drug moiety is selected from the group consisting of a 10-hydroxycamptothecin moiety and a 7-ethyl-10-hydroxycamptothecin moiety.

In one embodiment, the divalent moiety is substituted and unsubstituted alkyl.

In another embodiment, compounds of the invention have formula (II):

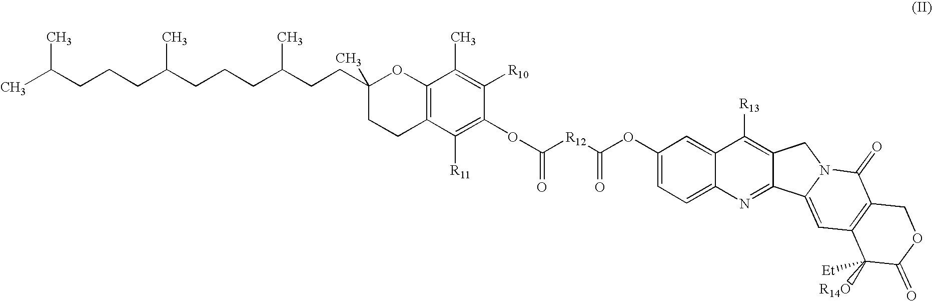

or a stereoisomer, tautomer, or pharmaceutically acceptable salt thereof,

wherein

R₁₀ is hydrogen or methyl;

R₁₁ is hydrogen or methyl;

R₁₂ is selected from the group consisting of:

(a) —(CH₂)ₙ—, where n is an integer from 3 to 12;

(b) —CH₂—CH(CH₃)—CH₂—;

(c) —CH₂—C(CH₂)ₘ—CH₂—, where m is an integer from 2 to 6;

(d) 1,3-phenylene;

(e) 1,4-phenylene;

(f) 1,2-cycloalkylene;

(g) 1,3-cycloalkylene; and

(h) 1,4-cycloalkylene;

R₁₃ is hydrogen or ethyl; and

R₁₄ is selected from the group consisting of hydrogen and —C(═O)—R₁₅, where R₁₅ is C1-C6 alkyl.

In one embodiment, R₁₀ and R₁₁ are methyl.

In one embodiment, R₁₀ and R₁₁ are hydrogen.

In one embodiment, R₁₂ is —(CH₂)ₙ— and n is an integer from 3 to 12. In one embodiment, R₁₂ is —(CH₂)ₙ— and n is 3.

In one embodiment, R₁₃ is ethyl.

In one embodiment, R₁₄ is hydrogen.

In one embodiment, the tocopherol moiety is a dl-α-tocopherol moiety.

In a further embodiment, compounds of the invention have formula (III):

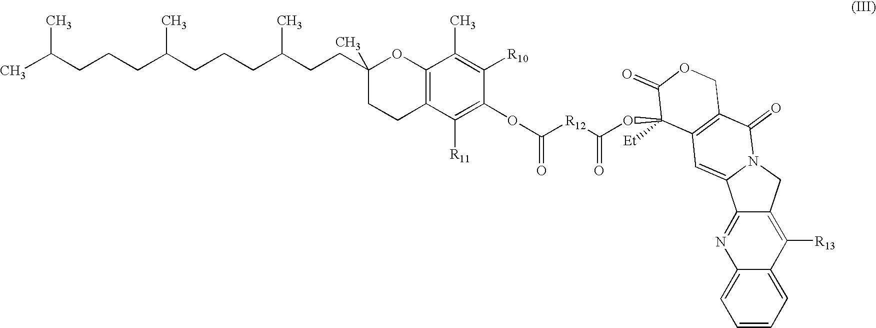

or a stereoisomer, tautomer, or pharmaceutically acceptable salt thereof,

wherein

R₁₀ is hydrogen or methyl;

R₁₁ is hydrogen or methyl;

R₁₂ is selected from the group consisting of:

(a) —(CH₂)ₙ—, where n is an integer from 3 to 12;

(b) —CH₂—CH(CH₃)—CH₂—;

(c) —CH₂—C(CH₂)ₘ—CH₂—, where m is an integer from 2 to 6;

(d) 1,3-phenylene;

(e) 1,4-phenylene;

(f) 1,2-cycloalkylene;

(g) 1,3-cycloalkylene; and

(h) 1,4-cycloalkylene; and

R₁₃ is hydrogen or ethyl.

In one embodiment, R₁₀ and R₁₁ are methyl.

In one embodiment, R₁₀ and R₁₁ are hydrogen.

In one embodiment, R₁₂ is —(CH₂)ₙ— and n is an integer from 3 to 12. In one embodiment, R₁₂ is —(CH₂)ₙ— and n is 3.

In one embodiment, R₁₃ is hydrogen.

In one embodiment, the tocopherol moiety is a dl-α-tocopherol moiety.

The preparations of representative diester-linked compounds are provided in Examples 20-47. In vitro cytotoxicities of certain of these representative compounds are compared in Example 48.

In another aspect, the present invention provides compositions that include the compounds of the invention. The compositions include one or more compounds of the invention, optionally one or more additional therapeutic agents, and a lipophilic medium. In one embodiment, a tocopherol-modified therapeutic drug compound is dissolved in the lipophilic medium. Because of the lipophilic moiety, the compound has improved lipophilicity compared to the unmodified therapeutic drug compound. The lipophilic medium (or carrier) of the composition can be any one of a variety of lipophilic mediums including, for example, oils. In one embodiment, the lipophilic medium includes a tocopherol (e.g., α-tocopherol). Representative oils useful as the lipophilic medium include the following:

Fatty acids and esters thereof, including carboxylic acids of various chain lengths, mostly straight chain, but which could be branched, examples of which include capric, caprylic, caproic, lauric, myristic, stearic, oleic, linoleic, behenic, and as well as saturated or unsaturated fatty acids and esters;

Fatty acids esterified with glycerin to form mono-, di-, or triglycerides, which can be synthetic or derived from natural sources, including, but not limited to, for example, glycerides such as soybean oil, cottonseed oil, rapeseed oil, fish oil, castor oil, Capmul MCM, Captex 300, Miglyol 812, glyceryl monooleate, triacetin, acetylated monoglyceride, tristearin, glyceryl behenate, and diacetyl tartaric acid esters of monoglycerides;

Glycerides conjugated to other moieties, such as polyethylene glycol (for example, Labrasol, Labrafac, Cremophor EL);

Phospholipids, either natural or synthetic, such as dimyristyl phosphatidylcholine, egg lecithin, and pegylated phospholipids;

Other fatty esters including fatty alcohols (myristyl myristate, isopropyl palmitate), or sugars (sorbitan monooleate, SPAN 80, Tween 80, sucrose laurate);

Fatty alcohols such as stearyl alcohol, lauryl alcohol, benzyl alcohol, or esters or ethers thereof, such as benzyl benzoate;

Fat-soluble vitamins and derivatives, for example, vitamin E (including all of the tocopherols and tocotrienols, and tocopherol and tocotrienol derivatives, such as vitamin E succinate, vitamin E acetate, and vitamin E succinate polyethylene glycol (TPGS)).

Organic co-solvents can also be used in the compositions, optionally in combination with water, including for example, ethanol, polyethylene glycol, propylene glycol, glycerol, N-methylpyrrolidone, and dimethyl sulfoxide.

The solubilities of two representative tocopherol-modified camptothecin compounds of the invention in several mediums are compared to camptothecin in Example 14.

In a further aspect, the invention provides emulsion, microemulsion, and micelle formulations that include a compound of the invention. Methods for making the emulsion, microemulsion, and micelle formulations are also provided.

As used herein, the term “emulsion” refers to a colloidal dispersion of two immiscible liquids, such as an oil and water, in the form of droplets, whose diameter, in general, are between 0.1 and 3.0 microns and which is typically optically opaque, unless the dispersed and continuous phases are refractive index matched. Such systems possess a finite stability, generally defined by the application or relevant reference system, which may be enhanced by the addition of amphiphilic molecules or viscosity enhancers.

The term “microemulsion” refers to a thermodynamically stable isotropically clear dispersion of two immiscible liquids, such as an oil and water, stabilized by an interfacial film of surfactant molecules. The microemulsion has a mean droplet diameter of less than 200 nm, in general between 10-50 nm. In the absence of water, mixtures of oil(s) and non-ionic surfactant(s) form clear and isotropic solutions that are known as self-emulsifying drug delivery systems (SEDDS) and can be used to improve lipophilic drug dissolution and oral absorption.

The emulsion and microemulsion formulations include an oil phase and an aqueous phase. The emulsion or microemulsion can be an oil-in-water emulsion or a water-in-oil emulsion. The oil phase includes one or more compounds of the invention and a lipophilic medium, as described above. In one embodiment, the compound is present in the formulation in an amount from about 0.005 to about 3.0 weight percent based on the total weight of the formulation. In one embodiment, the compound is present in the formulation in an amount from about 0.01 to about 2.5 weight percent based on the total weight of the formulation. In one embodiment, the compound is present in the formulation in an amount from about 0.1 to about 1.5 weight percent based on the total weight of the formulation. In one embodiment, the lipophilic medium is present in the formulation in an amount from about 2 to about 20 weight percent based on the total weight of the formulation. In one embodiment, the lipophilic medium is present in the formulation in an amount from about 4 to about 12 weight percent based on the total weight of the formulation. In one embodiment, the lipophilic medium is present in the formulation in an amount from about 6 to about 10 weight percent based on the total weight of the formulation.

In one embodiment of the emulsion or microemulsion, the compound is a tocopherol-modified therapeutic drug compound, the lipophilic medium includes a tocopherol, and the aqueous medium is water.

In addition to the compounds of the invention, the emulsion or microemulsion formulations can include other components commonly used in emulsions an microemulsions, and particularly used in pharmaceutical emulsions and microemulsions. These components include surfactants and co-solvents, among others. Representative surfactants include nonionic surfactants such as surface active tocopherol derivatives and surface active polymers.

Suitable surface active tocopherol derivatives include tocopherol polyethylene glycol derivatives, such as vitamin E succinate polyethylene glycol (e.g., d-α-tocopherol polyethylene glycol 1000 succinate, TPGS), which is a vitamin E derivative in which a polyethylene glycol is attached by a succinic acid ester at the ring hydroxyl of vitamin E. As used herein, “vitamin E succinate polyethylene glycol” includes vitamin E succinate polyethylene glycol and derivatives of vitamin E polyethylene glycol having various ester and ether links. TPGS is a non-ionic surfactant (HLB=16-18). TPGS is reported to inhibit P-glycoprotein, a protein that contributes to the development of multi-drug resistance. Embodiments of the formulations of the invention that include TPGS therefore include a P-glycoprotein inhibitor. Surface active tocopherol derivatives (e.g., TPGS) can be present in the formulations of the invention in an amount from about 1 to about 10 weight percent, about 2 to about 6 weight percent, or about 5 weight percent, based on the total weight of the formulation.

Suitable nonionic surfactants include block copolymers of ethylene oxide and propylene oxide known as POLOXAMERS or PLURONICS. These synthetic block copolymers of having the general structure: H(OCH₂CH₂)ₐ(OC₃H₆CH₂)_{b} (OCH₂CH₂)ₐOH. The following variants based on the values of a and b are commercially available from BASF Performance Chemicals (Parsippany, N.J.) under the trade name PLURONIC and consist of the group of surfactants designated by the CTFA name of POLOXAMER 108, 188, 217, 237, 238, 288, 338, 407, 101, 105, 122, 123, 124, 181, 182, 183, 184, 212, 231, 282, 331, 401, 402, 185, 215, 234, 235, 284, 333, 334, 335, and 403. For the most commonly used POLOXAMERS 124, 188, 237, 338, and 407 the values of a and b are 12/20, 79/28, 64/37, 141/44 and 101/56, respectively. In one embodiment the nonionic surfactant is present in the formulation in an amount from about 0.5 to about 5 weight percent based on the total weight of the formulation.

Co-solvents useful in the formulations include ethanol, polyethylene glycol, propylene glycol, glycerol, N-methylpyrrolidone, dimethylamide, and dimethylsulfoxide, among others. Polyethylene glycol (PEG) is a hydrophilic, polymerized form of ethylene glycol, consisting of repeating units having the chemical structure: (—CH₂CH₂O—). The general formula for polyethylene glycol is H(OCH₂CH₂)ₙOH. The molecular weight ranges from 200 to 10,000. Such various forms are described by their molecular weights, for example, PEG-200, PEG-300, PEG-400, and the like.

Paclitaxel emulsions and their components are described in U.S. Pat. No. 6,458,173 and U.S. Pat. No. 6,660,286, each expressly incorporated herein by reference in its entirety.

Representative emulsions including tocopherol-modified therapeutic drug compounds (e.g., tocopherol succinate docetaxel, tocopherol succinate paclitaxel, tocopherol succinate camptothecin, tocopherol succinate 7-ethyl-10-hydroxycamptothecin, and tocopherol succinate 10-hydroxycamptothecin) are described in Example 15. In vitro cytotoxicities of representative tocopherol-modified therapeutic drug compounds (e.g., tocopherol succinate 7-ethyl-10-hydroxycamptothecin and tocopherol succinate camptothecin) are described in Example 16.

In a further aspect, the invention provides micelle formulations that include a compound of the invention and an aqueous phase. Micelles are organized aggregates of one or more surfactants in solution. In one embodiment, the compound is present in the formulation in an amount from about 0.005 to about 3.0 weight percent based on the total weight of the formulation. In one embodiment, the compound is present in the formulation in an amount from about 0.01 to about 2.5 weight percent based on the total weight of the formulation. In one embodiment, the compound is present in the formulation in an amount from about 0.1 to about 1.0 weight percent based on the total weight of the formulation. Suitable surfactants include those noted above, and in the amounts noted above. In one embodiment of the micelle formulation, the compound is a tocopherol-modified therapeutic drug compound and the surfactant is tocopherol polyethylene glycol succinate (TPGS). Representative micelle formulations including tocopherol-modified therapeutic drug compounds are described in Example 15.

The micelle formulation can also include additional components such as co-solvents including those noted above. In one embodiment, the micelle formulation includes a polyethylene glycol and a lower alkyl alcohol (e.g., ethanol). In one embodiment, the co-solvents are present in an amount from about 2 to about 20 weight percent based on the total weight of the formulation. The micelle, emulsion, and microemulsion formulations include an aqueous phase. In one embodiment, the aqueous phase includes deionized water. In another embodiment, the aqueous phase includes saline. In another embodiment, the aqueous phase is saline buffered with an organic acid (e.g., succinate, citrate).

The invention also provides the use of the compounds of the invention in the manufacture of a medicament. For example, for compounds of the invention that include a therapeutic drug moiety derived from a therapeutic drug compound effective in treating cell proliferative disease, the invention provides the use of such compounds in the manufacture of a medicament for the treatment of cell proliferative disease.

In other aspects, methods for administering a compound of the invention to a subject in need thereof, and methods for treating a condition treatable by administration of a therapeutically effective amount of a compound of the invention are also provided. These methods include the administration of the compounds, compositions, emulsion formulations, microemulsion formulations, and micelle formulations described herein.

In one embodiment, the invention provides a method for treating a condition that is treatable by the parent, unmodified therapeutic drug compound (e.g., a cell proliferative disease such as cancer). In the method, a therapeutically effective amount of a compound of the invention is administered to a subject in need thereof.

In one embodiment, the invention provides a method for treating a cell proliferative disease by administering a compound of the invention having a therapeutic drug moiety derived from a therapeutic drug effective in treating cell proliferative disease. Representative cell proliferative diseases treatable by the compounds of the invention include hematologic cancers, such as leukemia, lymphoma, and myeloma; and nonhematologic cancers, such as solid tumor carcinomas (e.g., breast, ovarian, pancreatic, colon, colorectal, non-small cell lung, and bladder), sarcomas, and gliomas.

Therapeutically effective amounts of the compounds will generally range up to the maximally tolerated dosage, but the concentrations are not critical and may vary widely. The precise amounts employed by the attending physician will vary, of course, depending on the compound, route of administration, physical condition of the patient and other factors. The daily dosage may be administered as a single dosage or may be divided into multiple doses for administration.

The amount of the compound actually administered will be a therapeutically effective amount, which term is used herein to denote the amount needed to produce a substantial beneficial effect. Effective doses may be extrapolated from dose-response curves derived from in vitro or animal model test systems. The animal model is also typically used to determine a desirable dosage range and route of administration. Such information can then be used to determine useful doses and routes for administration in humans or other mammals. The determination of an effective dose is well within the capability of those skilled in the art. Thus, the amount actually administered will be dependent upon the individual to which treatment is to be applied, and will preferably be an optimized amount such that the desired effect is achieved without significant side-effects.

Therapeutic efficacy and possible toxicity of the compounds of the invention can be determined by standard pharmaceutical procedures, in cell cultures or experimental animals (e.g., ED₅₀, the dose therapeutically effective in 50% of the population; and LD₅₀, the dose lethal to 50% of the population). The dose ratio between therapeutic and toxic effects is the therapeutic index, and it can be expressed as the ratio LD₅₀ to ED₅₀. Modified therapeutic drug compounds that exhibit large therapeutic indices are particularly suitable in the practice of the methods of the invention. The data obtained from cell culture assays and animal studies may be used in formulating a range of dosage for use in humans or other mammals. The dosage of such compounds lies preferably within a range of circulating concentrations that include the ED₅₀ with little or no toxicity. The dosage typically varies within this range depending upon the dosage form employed, sensitivity of the patient, and the route of administration. Thus, optimal amounts will vary with the method of administration, and will generally be in accordance with the amounts of conventional medicaments administered in the same or a similar form.

The compounds of the invention can be administered alone, or in combination with one or more additional therapeutic agents. For example, in the treatment of cancer, the compounds can be administered in combination with therapeutic agents including, but not limited to, androgen inhibitors, such as flutamide and luprolide; antiestrogens, such as tomoxifen; antimetabolites and cytotoxic agents, such as daunorubicin, fluorouracil, floxuridine, interferon alpha, methotrexate, plicamycin, mercaptopurine, thioguanine, adriamycin, carmustine, lomustine, cytarabine, cyclophosphamide, doxorubicin, estramustine, altretamine, hydroxyurea, ifosfamide, procarbazine, mutamycin, busulfan, mitoxantrone, carboplatin, cisplatin, streptozocin, bleomycin, dactinomycin, and idamycin; hormones, such as medroxyprogesterone, estramustine, ethinyl estradiol, estradiol, leuprolide, megestrol, octreotide, diethylstilbestrol, chlorotrianisene, etoposide, podophyllotoxin, and goserelin; nitrogen mustard derivatives, such as melphalan, chlorambucil, methlorethamine, and thiotepa, steroids, such as betamethasone; and other antineoplastic agents, such as live Mycobacterium bovis, dicarbazine, asparaginase, leucovorin, mitotane, vincristine, vinblastine, and taxotere. Appropriate amounts in each case will vary with the particular agent, and will be either readily known to those skilled in the art or readily determinable by routine experimentation.

Administration of the compounds of the invention is accomplished by any effective route, for example, parenteral, topical, or oral routes. Methods of administration include inhalational, buccal, intramedullary, intravenous, intranasal, intrarectal, intraocular, intraabdominal, intraarterial, intraarticular, intracapsular, intracervical, intracranial, intraductal, intradural, intralesional, intramuscular, intralumbar, intramural, intraocular, intraoperative, intraparietal, intraperitoneal, intrapleural, intrapulmonary, intraspinal, intrathoracic, intratracheal, intratympanic, intrauterine, intravascular, and intraventricular administration, and other conventional means. The compounds of the invention having anti-tumor activity can be injected directly into a tumor, into the vicinity of a tumor, or into a blood vessel that supplies blood to the tumor.

The emulsion, microemulsion, and micelle formulations of the invention can be nebulized using suitable aerosol propellants that are known in the art for pulmonary delivery of the compounds.

The compounds of the invention may be formulated into a composition that additionally comprises suitable pharmaceutically acceptable carriers, including excipients and other compounds that facilitate administration of the compound to a subject. Further details on techniques for formulation and administration may be found in the latest edition of “Remington's Pharmaceutical Sciences” (Maack Publishing Co., Easton, Pa.).

Compositions for oral administration may be formulated using pharmaceutically acceptable carriers well known in the art, in dosages suitable for oral administration. Such carriers enable the compositions containing the compounds of the invention to be formulated as tablets, pills, dragees, capsules, liquids, gels, syrups, slurries, suspensions, suitable for ingestion by a subject. Compositions for oral use may be formulated, for example, in combination with a solid excipient, optionally grinding the resulting mixture, and processing the mixture of granules, after adding suitable additional compounds, if desired, to obtain tablets or dragee cores. Suitable excipients include carbohydrate or protein fillers. These include, but are not limited to, sugars, including lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins, such as gelatin and collagen. If desired, disintegrating or solubilizing agents may be added, such as the crosslinked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage).

Compounds for oral administration may be formulated, for example, as push-fit capsules made of gelatin, as well as soft, sealed capsules made of gelatin and a coating such as glycerol or sorbitol. Push-fit capsules may contain the compounds mixed with filler or binders such as lactose or starches, lubricants such as talc or magnesium stearate, and, optionally, stabilizers. In soft capsules, the covalent conjugates may be dissolved or suspended in suitable liquids, such as fatty oils, liquid paraffin, or liquid polyethylene glycol with or without stabilizers.

For topical or nasal administration, penetrants appropriate to the particular barrier to be permeated are typically used in the formulation. Examples of these are 2-pyrrolidone, N-methyl-2-pyrrolidone, dimethylacetamide, dimethyl-formamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface-active agents. Keratolytic agents such as those known in the art may also be included. Examples are salicylic acid and sulfur. For topical administration, the composition may be in the form of a transdermal ointment or patch for systemic delivery of the compound and may be prepared in a conventional manner (see, e.g., Barry, Dermatological Formulations (Drugs and the Pharmaceutical Sciences—Dekker); Harry's Cosmetology (Leonard Hill Books).

For rectal administration, the compositions may be administered in the form of suppositories or retention enemas. Such compositions may be prepared by mixing the compounds with a suitable non-irritating excipient that is solid at ordinary temperatures but liquid at the rectal temperature and will therefore melt in the rectum to release the drug. Suitable excipients include, but are not limited to, cocoa butter and polyethylene glycols.

The amounts of each of these various types of additives will be readily apparent to those skilled in the art, optimal amounts being the same as in other, known formulations designed for the same type of administration.

Compositions containing the compounds of the invention may be manufactured in a manner similar to that known in the art (e.g., by means of conventional mixing, dissolving, granulating, dragee-making, levigating, emulsifying, encapsulating, entrapping or lyophilizing processes). The compositions may also be modified to provide appropriate release characteristics, sustained release, or targeted release, by conventional means (e.g., coating). As noted above, in one embodiment, the compounds are formulated as an emulsion.

Compositions containing the compounds may be provided as a salt and can be formed with many acids, including but not limited to hydrochloric, sulfuric, acetic, lactic, tartaric, malic, and succinic. Salts tend to be more soluble in aqueous or other protonic solvents than are the corresponding free base forms.

After compositions formulated to contain a compound and an acceptable carrier have been prepared, they can be placed in an appropriate container and labeled for use. Thus, in another aspect, the invention provides kits.

Tocopherol-modified therapeutic drug compounds of the invention are suitable for administration as oil-in-water emulsions and micelle formulations. The compounds provide for high drug loading to enable small volumes for administration.

Emulsions containing tocopherol-modified camptothecin compounds of the invention provide for enhanced stability of the compound's lactone compared to conventional methods of camptothecin administration. Long plasma half-life is achieved for the tocopherol-modified camptothecin compounds resulting in prolonged exposure of a tumor to the compounds. Tocopherol-modified compounds achieve high permeation through lipoidal membranes of tumor cells. Greater anti-tumor response without an increase in toxicity may be provided by the tocopherol-modified camptothecin compounds of the invention as compared to unmodified camptothecin and currently available camptothecin analogs.

Although the compounds of the invention having formula (2) with m=1, formula (3) with n=1, or formula (8) do not include compounds specifically excluded as described above, it will be appreciated that the compositions, emulsion formulations, microemulsion formulations, and micelle formulations include the compounds of the invention having formulae (1)-(8) without such limitation. Methods for administering the compositions, emulsion formulations, microemulsion formulations, and micelle formulations, and methods for treating a condition treatable by administering the compositions, emulsion formulations, microemulsion formulations, and micelle formulations are likewise not limited with regard to the compounds of the invention.

The following examples are provided to illustrate, not limit, the invention.

### EXAMPLES

### Example 1

### The Preparation of a Representative Tocopherol-Modified Camptothecin Compound Tocopherol Succinate Camptothecin

A 500 ml flask was charged with 10.6 grams of d-α-tocopherol succinic acid, 6.97 grams of camptothecin, 6.13 grams of 2-chloro-1-methylpyridinium iodide (CMPI), 5.86 grams of 4-(dimethylamino)pyridine (DMAP), and 200 ml of dry N,N-dimethylacetamide. The mixture was stirred at room temperature for 24 hours, and then heated at 50° C. for 4 hours. The mixture was cooled to room temperature and then was filtered to remove precipitate and the filtrate was collected. To the filtrate were added 250 ml of chloroform and 150 ml of deionized-water to extract the product into the chloroform, and the water fraction was removed using a separation funnel. The chloroform fraction was washed with deionized-water (3×150 ml) in a separation funnel, collected, and dried over anhydrous MgSO₄ overnight. The MgSO₄ was removed by filtration, and the chloroform was removed with a rotary evaporator under reduced pressure to yield a dark-yellow solid. The product was purified by column chromatography on silica gel. (Yield: 9.50 grams, 55.2%).

¹H NMR (300 MHz, CDCl₃): δ 8.318 (s, 1H), 8.163-8.135 (d, J=8.4 Hz, 1H), 7.927-7.901 (d, J=7.8 Hz, 1H), 7.842-7.787 (m, 1H), 7.682-7.632 (m, 1H), 7.263-7.242 (d, J=6.3 Hz, 1H), 5.702-5.410 (ABq, J₁=17.4, J₂=70 Hz, 2H), 5.190 (s, 2H), 3.014-2.938 (m, 4H), 2.368-0.809 (m, 54H).

Elemental anal. Calcd. for C₅₃H₆₈N₂O₈: C, 73.92; H, 7.96; N, 3.25.

Found: C, 73.61; H, 7.90; N, 3.17.

### Example 2

### The Preparation of a Representative Tocopherol-Modified Camptothecin Compound Tocopherol Succinate 10-Hydroxycamptothecin

Method 1. A 100 ml flask was charged with 1.06 grams of d-α-tocopherol succinic acid, 0.476 grams of thionyl chloride, and 50 ml of toluene. The mixture was stirred at room temperature overnight. The solvent was removed with a rotary evaporator at 50° C., and the residue was collected. To the residue was added 0.728 grams of 10-hydroxycamptothecin and 40 ml of dried tetrahydrofuran with stirring. Then, 0.404 grams of triethylamine in 10 ml of tetrahydrofuran was added dropwise to the reaction mixture. The mixture was stirred at room temperature overnight. The mixture was filtered and white powder was washed with ethyl acetate (3×10 ml). The filtrate was collected. The solvent was removed with a rotary evaporator. The residue was collected, and purified by column chromatography on silica gel with a mobile phase of acetone and chloroform (1:4, v/v). (Yield: 0.85 grams, 48.4%).

MS (Positive ESI): m/z 877 (M)⁺.

Anal. Calcd. for C₅₃H₆₈N₂O₉: C, 72.58; H, 7.81; N, 3.19. Found: C, 72.52; H, 7.84; N, 3.21.

Alternatively, tocopherol succinate 10-hydroxycamptothecin can be prepared as described below.

Method 2. A 100 ml flask was charged with 2.65 grams of d-α-tocopherol succinate, 0.89 grams of thionyl chloride, and 20 ml of toluene. The mixture was stirred at room temperature for 24 hours. The toluene and any excess thionyl chloride were removed with vacuum distillation at 50° C. The remaining residue was dissolved in 15 ml of chloromethane to provide Solution A. To a 100 ml flask, 0.9 grams of 10-hydroxycamptothecin, 0.5 ml of triethylamine, and 25 ml of freshly dried N,N-dimethylacetamide was added with stirring. Then 15 ml of Solution A was slowly added into the mixture through a dropping funnel over 5 minutes. The reaction mixture was stirred at room temperature for 24 hours. The mixture was concentrated by vacuum distillation. 150 ml of ethyl acetate was added to the residue. The mixture was washed with saturated aqueous NaCl solution, (3<100 ml). The mixture was dried over anhydrous MgSO₄. The MgSO₄ was removed by filtration and the ethyl acetate was then removed by vacuum distillation. The crude product was purified by column chromatography on silica gel. (Yield: 1.14 grams, 52.5%).

### Example 3

### The Preparation of a Representative Tocopherol-Modified Camptothecin Compound: Tocopherol Succinate 7-Ethyl-10-hydroxycamptothecin

Method 1. A 500 ml flask was charged with 22.5 grams of d-α-tocopherol succinate, 7.6 grams of thionyl chloride, and 200 ml of toluene. The mixture was stirred at room temperature for 24 hours. The toluene and the excess thionyl chloride were removed by vacuum distillation. The remaining residue was dissolved in 100 ml of chloromethane to provide Solution A. Solution A was used immediately, and was not exposed to air. To a 500 ml flask, 7.8 grams of 7-ethyl-10-hydroxycamptothecin, 7 ml of triethylamine, and 250 ml of freshly dried N,N-dimethylacetamide was added with stirring. The 100 ml of Solution A was slowly added into the mixture through a dropping funnel over 30 minutes. The reaction mixture was stirred at room temperature for 24 hours. The solvent was concentrated by vacuum distillation. 500 ml of ethyl acetate was added to the residue. The mixture was washed with saturated aqueous NaCl solution, (3×200 ml). The mixture was dried over anhydrous MgSO₄. The MgSO₄ was removed by filtration and the ethyl acetate was then removed by vacuum distillation. The crude product was purified by recrystallization with acetone. (Yield: 15.18 grams, 83.9%).

M.P. 171°-173° C.

¹H NMR (300 MHz, CDCl₃): δ 8.236-8.206 (d, J=9 Hz, 1H), 7.809-7.801 (d, J=2.4 Hz, 1H), 7.648 (s, 1H), 7.572-7.533 (dd, J₁=2.7 Hz, J₂=9.3 Hz, 1H), 5.781-5.280 (ABq, J₁=16.2 Hz, J₂=134.0 Hz, 2H), 5.253 (s, 2H), 3.863 (s, 1H), 3.136-3.113 (m, 6H), 2.588 (t, 2H), 2.091 (s, 3H), 2.037 (s, 3H), 1.994 (s, 3H), 1.970-1.852 (m, 2H), 1.821-1.725 (m, 2H), 1.654-0.833 (m, 42H).

MS (Positive ESI): m/z 905 (M)⁺, 928 (M+Na)⁺.

Anal. Calcd. for C₅₅H₇₂N₂O₉: C, 72.98; H, 8.02; N, 3.09. Found: C, 72.87; H, 8.01; N, 2.88.

Alternatively, the tocopherol succinate 7-ethyl-10-hydroxycamptothecin can be prepared as described below.

Method 2. A 500 ml flask was charged with 8.48 grams of d-α-tocopherol succinate, 3.81 grams of thionyl chloride, and 250 ml of toluene. The mixture was stirred at room temperature overnight. The toluene, and excess thionyl chloride were removed with a rotary evaporator at 50° C., and the residue was collected. To the residue was added 6.27 grams of 7-ethyl-10-hydroxycamptothecin and 250 ml of sodium-dried tetrahydrofuran with stirring. Then, 3.23 grams of triethylamine in 50 ml of tetrahydrofuran was added dropwise to the mixture. The mixture was stirred at room temperature overnight. The mixture was filtered and the white powder was washed with ethyl acetate (3×50 ml). The filtrate was collected. The solvent was removed with a rotary evaporator. The crude product was purified by recrystallization in acetone. (Yield: 8.28 grams, 57.2%).

### Example 4

### The Preparation of a Representative Tocopherol-Modified Camptothecin Compound: 10,20-Di(tocopherol succinate) 7-Ethyl-10-hydroxycamptothecin

A 100 ml flask was charged with 0.905 grams of tocopherol succinate 7-ethyl-10-hydroxycamptothecin, 0.53 grams of d-α-tocopherol succinic acid, 0.255 grams of 2-chloro-1-methylpyridinium iodide, 0.244 grams of 4-(dimethylamino)pyridine and 50 ml of dioxane. The mixture was stirred at room temperature for 24 hours. Thin layer chromatography showed that the reaction was complete. The mixture was filtered to remove the solid phase, and the filtrate was collected. The solvent was removed by vacuum distillation. The crude product was purified by column chromatography on silica gel with 30% ethyl acetate in cyclohexane. (Yield: 0.64 grams, 44.82%).

¹H NMR (300 MHz, CDCl₃): δ 8.168-8.138 (d, J=9.0 Hz, 1H), 7.813-7.805 (d, J=2.4 Hz, 1H), 7.754-7.536 (dd, J₁=2.1 Hz, J₂=11.4 Hz, 1H), 7.197 (s, 1H), 5.703-5.409 (ABq, J₁=17.4 Hz, J₂=71.0 Hz, 2H), 5.243-5.088 (m, 2H), 3.113-2.857 (m, 10H), 2.606-2.564 (t, J=6 Hz, 2H), 2.383-2.184 (m, 2H), 2.090-1.723 (m, 22H), 1.588-0.785 (m, 80H).

MS (Positive ESI): m/z 1418 (M+H)⁺.

Elemental anal. Calcd. for C₈₈H₁₂₄N₂O₁₃: C, 74.54; H, 8.81; N, 1.98.

Found: C, 74.31; H, 8.96; N, 1.75.

IRνₘₐₓ^{KBr} cm⁻¹: 2925, 2867, 1751, 1665, 1615, 1657, 1510, 1458, 1413, 1376, 1330, 1218, 1128, 1075, 1060, 1035, 992, 943, 923, 829, 812, 758, 724, 668.

### Example 5

### The Preparation of a Representative Tocopherol-Modified Camptothecin Compound: Tocopherol-Camptothecin Conjugate with a Hexa(ethylene glycol) Linker

Preparation of hexa(ethylene glycol) tocopherol succinate. In a 250 ml flask, 2.65 grams of d-α-tocopherol succinic acid and 2.82 grams of hexa(ethylene glycol) was dissolved in 100 ml of toluene with stirring. The toluene was removed with a rotary evaporator (drying by azeotropic distillation). To the mixture was added 100 ml of chloroform, 1.08 grams of N,N-dicyclohexylcarbodiimide, and 100 mg of 4-(dimethylamino)pyridine. The mixture was stirred overnight. Thin layer chromatography with 40% acetone in hexane showed that the reaction was complete. The mixture was washed three times with deionized-water (3×100 ml), and the chloroform fraction was collected, and dried over anhydrous MgSO₄ for two hours. After filtration, chloroform was removed by a rotary evaporator. The crude product was purified by column chromatography on silica gel using, successively, the solvents 30% ethyl acetate in hexane, 50% ethyl acetate in hexane, and 30% acetone in hexane. (Yield: 0.53 grams, 13.33%).

Preparation of tocopherol-succinyl-hexa(ethylene glycol) succinic acid. A 100 ml flask was charged with 1.42 grams of hexa(ethylene glycol) tocopherol succinate prepared above, 0.2 grams of succinic acid anhydride, 2 drops of tin (II) ethylhexanoate, and 25 ml of xylene. The mixture was refluxed for 4 hours. After the reaction was complete, the solvent was removed by a rotary evaporator. The crude product was purified by column chromatography on silica gel. (Yield: 0.864 grams, 54%).

Preparation of Tocopherol Succinate Camptothecin with a Hexa(Ethylene Glycol) linker. A 100 ml flask was charged with 0.822 grams of tocopherol-succinyl-hexa(ethylene glycol) succinic acid prepared above, 0.3 grams of camptothecin, 0.47 grams of 2-chloro-1-methylpyridinium iodide, 0.45 grams of 4-(dimethylamino)pyridine, and 40 ml of dried N,N-dimethylacetamide. The reaction mixture was stirred at room temperature overnight. After the reaction was complete, the solvent was removed by vacuum distillation, and the residue was collected. To the residue was added 100 ml of ethyl acetate. After stirring for 30 minutes, the mixture was filtered to remove precipitate, and the filtrate was collected and concentrated. The crude product was purified by column chromatography on silica gel. (Yield: 0.342 grams, 30.4%).

¹H NMR (300 MHz, CDCl₃): δ 8.388 (s, 1H), 8.240-8.213 (d, J=8.1 Hz, 1H), 7.951-7.923 (d, J=8.4 Hz, 1H), 7.861-7.805 (dt, J₁=1.5 Hz, J₂=8.4 Hz, 1H), 7.694-7.640 (dt, J₁=1.2 Hz, J₂=8.1 Hz, 1H), 7.269 (s, 1H), 5.708-5.365 (ABq, J₁=17.1 Hz, J₂ 85.8 Hz, 2H), 5.283 (s, 2H), 4.273-4.169 (m, 4H), 3.705-3.673 (t, 2H), 3.631-3.550 (m, 18H), 2.926-2.654 (m, 8H), 2.597-2.552 (t, J=6.9 Hz, 2H), 2.381-2.2.113 (m, 2H), 2.074 (s, 3H), 2.004 (s, 3H), 1.964 (s, 3H), 1.827-1.683 (m, 2H), 1.655 (s, 3H), 1.544-0.964 (m, 24H), 0.875-0.830 (m, 12H).

MS (Positive ESI): m/z 1225 (M)⁺.

Elemental anal. Calcd. for C₈₈H₁₂₄N₂O₁₃: C, 67.62; H, 7.90; N, 2.29.

Found: C, 67.08; H, 8.04; N, 2.07.

IRνₘₐₓ^{KBr} cm⁻¹: 2925, 2867, 1735, 1667, 1618, 1563, 1500, 1457, 1405, 1366, 1349, 1232, 1204 1141, 1107, 1060, 994, 945, 859, 813, 787, 761, 723, 707.

### Example 6

### The Preparation of a Representative Tocopherol-Modified Paclitaxel Compound Tocopherol Succinate Paclitaxel

A 250 ml flask was charged with 5.83 grams of tocopherol succinic acid, 2.38 grams of thionyl chloride, and 50 ml of toluene. The mixture was stirred at room temperature overnight. The solvent was removed with a rotary evaporator at 50° C., and the residue was collected. To the residue were added 8.54 grams of paclitaxel and 100 ml of dried tetrahydrofuran with stirring. Then, 1.52 grams of triethylamine in 50 ml of tetrahydrofuran was added dropwise to the reaction mixture. The mixture was stirred at room temperature overnight. The mixture was filtered and the white powder was washed with ethyl acetate (3×10 ml). The filtrate was collected. The solvent was removed with a rotary evaporator. The residue was collected, and purified by recrystallization in acetone and hexane. (Yield: 11.56 grams, 84.6%).

Anal. Calcd. for C₈₀H₁₀₃NO₁₈: C, 70.31; H, 7.59; N, 1.02. Found: C, 70.02, H, 7.83; N, 0.93.

### Example 7

### The Preparation of a Representative Tocopherol-Modified Docetaxel Compound Tocopherol Succinate Docetaxel

A 250 ml flask is charged with 9.86 grams of d-α-tocopherol succinic acid, 5.0 grams of docetaxel, 3.83 grams of dried N,N-dicyclohexylcarbodiimide, 500 mg of 4-(dimethylamino)pyridine, and 150 ml of chloroform. The mixture is stirred at room temperature overnight. The mixture is filtered to remove precipitate and the filtrate is collected. The solvent is removed with rotary evaporator, and the residue is collected. The crude product is purified by column chromatography on silica gel.

### Example 8

### The Preparation of Mono-Tocopherol Phthalate

A 100 mL flask was charged with 8.61 grams of dl-α-tocopherol, 2.96 grams of phthalic anhydride, 50 mg of tin (II) 2-ethylhexanoate, and 50 ml of dried N,N-dimethylacetamide. The mixture was stirred at about 140° C. for 24 hours. After the mixture was cooled to room temperature, the mixture was poured into 150 ml of ethyl acetate. The mixture was washed three times with saturated aqueous NaCl (3×100 ml), and dried over anhydrous MgSO₄ overnight. The crude product was purified by column chromatography on silica gel with 30% ethyl acetate in hexane. (Yield: 3.6 grams, 31.1%).

¹H NMR (300 MHz, CDCl₃) δ ppm: 10.80 (bs, 1H), 8.119-8.063 (m, 1H), 7.883-7.828 (m, 1H), 7.678-7.616 (m, 2H), 2.627-2.582 (t, 2H), 2.123 (s, 3H), 2.112 (s, 3H), 2.081 (s, 3H), 1.868-1.702 (m, 2H), 1.616-1.020 (m, 24H), 0.874-0.834 (m, 12H).

Anal. Calcd. for C₃₇H₅₄O₅: C, 76.78; H, 9.40. Found: C, 76.57; H, 9.29.

IRνₘₐₓ^{KBr} cm⁻¹: 3073, 2919, 2858, 1737, 1701, 1578, 1455, 1409, 1373, 1276, 1230, 1107, 1071, 913, 738.

### Example 9

### The Preparation of Mono-Tocopherol Terephthalate

A 100 mL flask was charged with 4.30 grams of dl-α-tocopherol, 3.32 grams of terephthalic acid, 2.55 grams of 2-chloro-1-methylpyridinium iodide, 0.244 grams of 4-(dimethylamino)pyridine, and 50 ml of dry N,N-dimethylacetamide. The mixture was stirred at 50° C. for 4 hours. Thin layer chromatography showed that the reaction was complete. After the mixture was cooled to room temperature, the mixture was poured into 150 ml of ethyl acetate. The mixture was washed three times with saturated aqueous NaCl (3×100 mL), and dried over anhydrous MgSO₄ overnight. The crude product was purified by column chromatography on silica gel with 30% ethyl ether in hexane. (Yield: 1.60 grams, 27.6%)

¹H NMR (300 MHz, CDCl₃) δ ppm: 11.80 (bs, 1H), 8.374-8.259 (q, J₁=8.4 Hz, J₂=26.1 Hz, 4H), 2.650-2.607 (t, 2H), 2.130 (s, 3H), 2.066 (s, 3H), 2.024 (s, 3H), 1.895-1.783 (m, 2H), 1.532-1.083 (m, 24H), 0.878-0.839 (m, 12H).

Anal. Calcd. for C₃₇H₅₄O₅: C, 76.78; H, 9.40. Found: C, 76.64; H, 9.39.

IRνₘₐₓ^{KBr} cm⁻¹: 3062, 2924, 2858, 1737, 1696, 1573, 1460, 1424, 1373, 1276, 1240, 1097, 928, 774, 723.

### Example 10

### The Preparation of a Representative Tocopherol-Modified Camptothecin Compound: Tocopherol Terephthalate Camptothecin

A 100 mL flask was charged with 1.16 grams of mono-tocopherol terephthalate prepared above, 0.70 grams of camptothecin, 0.511 grams of 2-chloro-1-methylpyridinium iodide, and 0.489 grams of 4-(dimethylamino)pyridine. The mixture was stirred at 50° C. overnight. Thin layer chromatography showed the reaction was complete. After the mixture cooled to room temperature, the reaction mixture was poured into 150 ml of ethyl acetate. The mixture was filtered and the filtrate was collected. The filtrate was washed with saturated aqueous NaCl (3<100 ml), and dried over anhydrous MgSO₄ overnight. The crude product was purified by column chromatography on silica gel. (Yield: 0.560 grams, 30.8%)

¹H NMR (300 MHz, CDCl₃) δ ppm: 8.387 (s, 1H), 8.370-8.242 (q, J₁=8.4 Hz, J₂=30.3 Hz, 4H), 8.167-8.139 (d, J=8.4 Hz, 1H), 7.937-7.910 (d, J=8.1 Hz, 1H), 7.823-7.774 (t, 1H), 7.672-7.625 (t, 1H), 7.260 (s, 1H), 5.823-5.462 (ABq, J₁=17.4 Hz, J₂=90.9 Hz, 2H), 5.302 (s, 2H), 2.461 (t, 2H), 2.559-2.312 (m, 2H), 2.123 (s, 3H), 2.056 (s, 3H), 2.015 (s, 3H), 1.844-1.801 (m, 2H), 1.629-1.085 (m, 27H), 0.938-0.789 (m, 12H).

Anal. Calcd. for C₅₇H₆₈N₂O₈: C, 75.30; H, 7.54; N, 3.08. Found: C, 74.91; H, 7.56; N, 3.02.

IRνₘₐₓ^{KBr} cm⁻¹: 3057, 2924, 2858, 1757, 1737, 1675, 1614, 1558, 1450, 1399, 1266, 1235, 1163, 1102, 1020, 723.

### Example 11

### The Preparation of a Representative Tocopherol-Modified Camptothecin Compound: Tocopherol Cyclohexane-1,2-dicarboxylate 7-Ethyl-10-hydroxycamptothecin

Preparation of tocopherol cyclohexane-1,2-dicarboxylic acid. A mixture of 1.54 grams of 1,2-cyclohexanedicarbolic acid anhydride, 8.6 grams of d-α-tocopherol, 1.34 grams of aluminum trichloride, and 100 ml of cyclohexane in a 250 ml flask was heated under reflux for about 30 minutes. After the mixture cooled to room temperature, it was filtered. The filtrate was washed with a dilute aqueous hydrochloric acid solution and then dried over anhydrous MgSO₄. The mixture was concentrated, and crude product was purified by column chromatography on silica gel. (Yield: 3.325 grams, 56.9%).

Preparation of tocopherol cyclohexane-1,2-dicarboxylate 7-ethyl-10-hydroxycamptothecin. A mixture of 1.08 grams of tocopherol cyclohexane-1,2-dicarboxylic acid prepared above, 0.44 grams of thionyl chloride, and 20 ml of toluene was stirred under nitrogen overnight. The toluene and excess thionyl chloride were removed by vacuum distillation, and the residue was dissolved in 10 ml of dichloromethane to provide Solution A. In a 100 ml flask, 0.350 grams of SN38 was dissolved in 25 ml of dried N,N-dimethylacetamide to provide Solution B. Solution A and 0.186 g of triethylamine were added to Solution B. The mixture was stirred overnight at room temperature. The crude product was purified by column chromatography on silica gel. (Yield: 0.59 grams, 68.9%).

### Example 12

### The Preparation of a Representative Tocopherol-Modified Doxorubicin Compound Tocopherol Succinate Doxorubicin

A 100 ml flask is charged with equivalent moles (1 mmole) of tocopherol succinic acid, doxorubicin, and N,N-dicyclohexylcarbodiimide, and 50 ml of dry N,N-dimethylacetamide. The mixture is stirred at room temperature until completion of the reaction. The mixture is filtered to remove white precipitate and the filtrate is collected. The solvent is removed with a rotary evaporator, and the residue is collected. The product is purified by either recrystallization or column chromatography on silica gel.

### Example 13

### The Preparation of a Representative Tocopherol-Modified Hydroxyzine Compound Tocopherol Succinate Hydroxyzine

A 100 ml flask is charged with equivalent moles (1 mmole) of tocopherol succinic acid, and thionyl chloride, and 50 ml of toluene. The mixture is stirred at room temperature overnight. The solvent is removed with a rotary evaporator at 50° C., and the residue is collected. To the residue are added 1 mmole of hydroxyzine and 40 ml of chloroform with stirring. Then, 1 mmole of triethylamine in 10 ml of chloroform is added dropwise to the reaction mixture at 0-5° C. The mixture is then stirred at room temperature overnight. The mixture is washed with saturated NaHCO₃ aqueous solution (3×50 ml). The organic phase is collected, and dried with anhydrous MgSO₄. The solvent is removed with a rotary evaporator after removal of MgSO₄. The residue is collected, and the crude product is purified by either recrystallization or silica column chromatography.

### Example 14

### Representative Tocopherol-Modified Therapeutic Drug Compound Solubility

In this example, the solubility of representative tocopherol-modified therapeutic drug compounds of the invention, tocopherol succinate camptothecin and tocopherol succinate 7-ethyl-10-hydroxycamptothecin, was compared to the solubility of camptothecin in a variety of solvents.

The solubility of camptothecin, tocopherol succinate camptothecin, and tocopherol succinate 7-ethyl-10-hydroxycamptothecin was determined in several solvents. Compounds were dissolved in each solvent under constant stirring and temperature to saturation. The resulting solutions were centrifuged and the supernatant was analyzed by high performance liquid chromatography (HPLC).

The comparative solubility (mg/g) of camptothecin, tocopherol succinate camptothecin, and tocopherol succinate 7-ethyl-10-hydroxycamptothecin in various solvents is shown in Table 1.

TABLE 1
Solubility Comparison of Camptothecin and Tocopherol Succinate
Camptothecins.

| | Camptothecin | VESA- | VESA-CPT² | Temperature |
|---|---|---|---|---|
| Solvent | (mg/g) | SN38¹ | (mg/g) | (° C.) |
| PEG-400 | — | 0.017 | 17.5 | Room Temp. |
| NF | | | | |
| TPGS | — | 27.6 | >133.3 | 65 |
| Vitamin E | 1.96 | 398.2 | >288.3 | 65 |
| USP/NF | | | | |
| Soybean Oil | 0.00 | 3.3 | 45.2 | Room Temp. |
| USP | | | | |
| Captex 300 | — | 4.4 | 96.7 | Room Temp. |
| EP | | | | |
| Tween 80 | — | 2.7 | 48.3 | 65 → Room |
| NF | | | | Temp. |
| Ethanol | — | 4.2 | 57.1 | Room Temp. |
| Denatured | | | | |
| Methanol | — | 3.8 | 14.4 | Room Temp. |
| Acetonitrile | 0.09 | 3.1 | 49.6 | Room Temp. |
| Chloroform | 0.71 | >97.0 | >372.5 | Room Temp. |
| DMSO | 50 | 30.5 | >255.5 | Room Temp. |
| Methylene | 0.9 | >99.7 | >336.5 | Room Temp. |
| dichloride | | | | |
| Propylene | — | 3.2 | 0.4633 | Room Temp. |
| Glycol, USP | | | | |
| Glycerin | — | 2.8 | 2.541 | Room Temp. |
| USP/EP/BP/JP | | | | |

¹VESA-SN38: tocopherol succinate 7-ethyl-10-hydroxycamptothecin
²VESA-CPT: tocopherol succinate camptothecin

The results in Table 1 illustrate that tocopherol succinate camptothecin and tocopherol succinate 7-ethyl-10-hydroxycamptothecin both have substantial solubility in oils, and have particularly high solubility in vitamin E (α-tocopherol).

### Example 15

### Representative Tocopherol-Modified Therapeutic Drug Compound-Containing Emulsions

In this example, representative emulsions containing tocopherol-modified therapeutic drug compounds of the invention are described.

A. Tocopherol Succinate 7-Ethyl-10-Hydroxycamptothecin Emulsion

Tocopherol succinate 7-ethyl-10-hydroxycamptothecin, prepared as described in Example 3, was dissolved in vitamin E and then emulsified with the use of a microfluidizer (M110Y Microfluidics) in the presence of d-α-tocopherol polyethylene glycol 1000 succinate (TPGS), Poloxamer 407, and saline to produce an emulsion having the following composition (% by weight):

| | |
|---|---|
| Tocopherol succinate-7-ethyl-10- | 0.69% |
| hydroxycamptothecin | |
| Vitamin E | 7.31% |
| TPGS | 5% |
| Poloxamer 407 | 1% |
| Saline | 86% |

The emulsion was filtered through a 0.2 μm filter and vialed in sterile glass vials. Mean particle size was approximately 50 nM as determined by submicron particle sizer (Nicomp Model 370), with 99% of the particles less than 80 nm. No evidence of precipitation or loss of concentration as measured by HPLC was observed for at least 3 months when stored at 4° C.

B. Tocopherol Succinate 7-Ethyl-10-hydroxycamptothecin Emulsion

Tocopherol succinate 7-ethyl-10-hydroxycamptothecin, prepared as described in Example 3, was dissolved in vitamin E and then emulsified with the use of a microfluidizer (M110Y Microfluidics) in the presence of TPGS and saline to produce an emulsion having the following composition (% by weight):

| | |
|---|---|
| Tocopherol succinate-7-ethyl-10- | 0.69% |
| hydroxycamptothecin | |
| Vitamin E | 7.31% |
| TPGS | 5% |
| Saline | 87% |

This formulation resulted in a more yellow and thicker emulsion than the emulsion prepared as described above that included Poloxamer 407. The emulsion was filtered through a 0.2 μm filter and vialed in sterile glass vials. Mean particle size was approximately 75 nm as determined by submicron particle sizer (Nicomp Model 370), with 99% of the particles less than 170 nm. No evidence of precipitation or loss of concentration as measured by HPLC was observed for at least 3 months when stored at

C. Tocopherol Succinate Camptothecin Emulsion

Tocopherol succinate camptothecin, prepared as described in Example 1, was dissolved in vitamin E and then emulsified with the use of a microfluidizer (M110Y Microfluidics) in the presence of TPGS, Poloxamer 407, and saline to produce an emulsion having the following composition (% by weight):

| | |
|---|---|
| Tocopherol succinate camptothecin | 0.74% |
| Vitamin E | 7.26% |
| TPGS | 5% |
| Poloxamer 407 | 1% |
| Saline | 86% |

The emulsion was filtered through a 0.2 μm filter and vialed in sterile glass vials. Mean particle size was approximately 40 nm as determined by submicron particle sizer (Nicomp Model 370), with 99% of the particles less than 75 nm. No evidence of precipitation or loss of concentration as measured by HPLC was observed for at least 3 months when stored at 4° C.

D. Tocopherol Succinate Camptothecin Emulsion

Tocopherol succinate camptothecin, prepared as described in Example 1, was dissolved in vitamin E and then emulsified with the use of a microfluidizer (M110Y Microfluidics) in the presence of TPGS, Poloxamer 407, and saline to produce an emulsion having the following composition (% by weight):

| | |
|---|---|
| Tocopherol succinate camptothecin | 1.48% |
| Vitamin E | 6.52% |
| TPGS | 5% |
| Poloxamer 407 | 1% |
| Saline | 86% |

The emulsion was filtered through a 0.2 μm filter and vialed in sterile glass vials. Mean particle size was approximately 30 nm as determined by submicron particle sizer (Nicomp Model 370), with 99% of the particles less than 100 nm. No evidence of precipitation or loss of concentration as measured by HPLC was observed for at least 3 months when stored at 4° C.

E. Tocopherol Succinate 7-Ethyl-10-hydroxycamptothecin Emulsion

Tocopherol succinate 7-ethyl-10-hydroxycamptothecin, prepared as described in Example 3, was dissolved in vitamin E and then emulsified with the use of a microfluidizer (M110Y Microfluidics) in the presence of TPGS and citric acid buffered saline to produce an emulsion having the following composition (% by weight):

| | |
|---|---|
| Tocopherol succinate-7-ethyl-10- | 0.69% |
| hydroxycamptothecin | |
| Vitamin E | 7.31% |
| TPGS | 5% |
| Citric acid buffered saline, pH 3.0 | 87% |

The emulsion was filtered through a 0.2 μm filter and vialed in sterile glass vials. Mean particle size was approximately 60 nm as determined by submicron particle sizer (Nicomp Model 370), with 99% of the particles less than 150 nm. No evidence of precipitation or loss of concentration as measured by HPLC was observed for at least 3 months when stored at 4° C. and 25° C.

F. Tocopherol Succinate 7-Ethyl-10-hydroxycamptothecin Emulsion

Tocopherol succinate 7-ethyl-10-hydroxycamptothecin, prepared as described in Example 3, was dissolved in vitamin E and then emulsified with the use of a microfluidizer (M110Y Microfluidics) in the presence of TPGS and succinate buffered saline to produce emulsions having the following composition (% by weight):

Formulation 1

| | |
|---|---|
| Tocopherol succinate-7-ethyl-10- | 0.69% |
| hydroxycamptothecin | |
| Vitamin E | 7.31% |
| TPGS | 5% |
| Succinate buffered saline, pH 4.0 | 87% |

The emulsion was filtered through a 0.2 μm filter and vialed in sterile glass vials. Mean particle size was approximately 70 nm as determined by submicron particle sizer (Nicomp Model 370), with 99% of the particles less than 170 nm. No evidence of precipitation or loss of concentration as measured by HPLC was observed for at least 3 months when stored at 4° C. and 25° C.

Formulation 2

| | |
|---|---|
| Tocopherol succinate-7-ethyl-10- | 1% |
| hydroxycamptothecin | |
| Vitamin E | 7% |
| TPGS | 5% |
| Succinate buffered saline, pH 4.0 | 87% |

The emulsion was filtered through a 0.2 μm filter and vialed in sterile glass vials. Mean particle size was approximately 70 nm as determined by submicron particle sizer (Nicomp Model 370), with 99% of the particles less than 170 nm. No evidence of precipitation or loss of concentration as measured by HPLC was observed for at least 1 month when stored at 4° C., 25° C., and 40° C.

Formulation 3

| | |
|---|---|
| Tocopherol succinate-7-ethyl-10- | 1% |
| hydroxycamptothecin | |
| Vitamin E | 6% |
| TPGS | 4% |
| Succinate buffered saline, pH 4.0 | 89% |

The emulsion was filtered through a 0.2 μm filter and vialed in sterile glass vials. Mean particle size was approximately 95 nm as determined by submicron particle sizer (Nicomp Model 370), with 99% of the particles less than 220 nm. No evidence of precipitation or loss of concentration as measured by HPLC was observed for at least 1 month when stored at 4° C., 25° C., and 40° C.

G. Tocopherol Succinate 7-Ethyl-10-hydroxycamptothecin (VESA-SN38) Micelle Formulation

Tocopherol succinate 7-ethyl-10-hydroxycamptothecin was dissolved in a mixture containing TPGS, PEG (300), and ethanol at about 50° C.-about 60° C. with stirring for about 1 hour to form a transparent solution. To this solution was added either deionized-water (DI-water), Poloxamer 407 and DI-water, Poloxamer 188 and DI-water, or 0.9% NaCl aqueous solution to form Formulations 1-5 respectively below. The formulations were stirred for a few minutes to form transparent micelle solutions having the following compositions (% by weight):

Formulation 1

| | |
|---|---|
| VESA-SN38 | 0.2% |
| TPGS | 5% |
| Ethanol | 5% |
| PEG(300) | 5% |
| DI-water | 84.8% |

The formulation solution was filtered through a 0.2 μm filter and vialed in sterile glass vials. No evidence of precipitation or loss of concentration as measured by HPLC was observed for at least 11 weeks when stored at 4° C.

Formulation 2

| | |
|---|---|
| VESA-SN38 | 0.2% |
| TPGS | 5% |
| Poloxamer 407 | 1.7% |
| Ethanol | 5% |
| PEG(300) | 5% |
| DI-water | 83.1% |

The formulation solution was filtered through a 0.2 μm filter and vialed in sterile glass vials. No evidence of precipitation or loss of concentration as measured by HPLC was observed for at least 11 weeks when stored at 4° C.

Formulation 3

| | |
|---|---|
| VESA-SN38 | 0.2% |
| TPGS | 5% |
| PEG(300) | 5% |
| Ethanol | 5% |
| Poloxamer 188 | 1.7% |
| DI-water | 83.1% |

The formulation solution was filtered through a 0.2 μm filter and vialed in sterile glass vials. No evidence of precipitation or loss of concentration as measured by HPLC was observed for at least 11 weeks when stored at 4° C. and 25° C.

Formulation 4

| | |
|---|---|
| VESA-SN38 | 0.2% |
| TPGS | 2% |
| PEG(300) | 2% |
| Ethanol | 4% |
| Saline | 91.8% |

The formulation solution was filtered through a 0.2 μm filter and vialed in sterile glass vials. No evidence of precipitation or loss of concentration as measured by HPLC was observed for at least 1 week when stored at 4° C., 25° C., or 40° C.

Formulation 5

| | |
|---|---|
| VESA-SN38 | 0.5% |
| TPGS | 5% |
| PEG(300) | 5% |
| Ethanol | 10% |
| Saline | 79.5% |

The formulation solution was filtered through a 0.2 μm filter and vialed in sterile glass vials. No evidence of precipitation or loss of concentration as measured by HPLC was observed for at least 3 weeks when stored at 4° C.

H. Tocopherol Succinate 7-Ethyl-10-hydroxycamptothecin (VESA-SN38) Micelle Formulation

Tocopherol succinate 7-ethyl-10-hydroxycamptothecin was dissolved in a mixture containing TPGS, PEG(300), and ethanol at about 50° C.-about 60° C. with stirring for about 1 hour to form a transparent solution. To this solution was added succinate buffered saline to form Formulations 1 and 2 below. The formulations were stirred for a few minutes to form transparent micelle solutions having the following compositions (% by weight):

Formulation 1

| | |
|---|---|
| VESA-SN38 | 0.2% |
| TPGS | 2% |
| Ethanol | 4% |
| PEG(300) | 2% |
| Succinate buffered saline, pH 4.0 | 91.8% |

The formulation solution was filtered through a 0.2 μm filter and vialed in sterile glass vials.

Formulation 2

| | |
|---|---|
| VESA-SN38 | 0.5% |
| TPGS | 5% |
| Ethanol | 10% |
| PEG(300) | 5% |
| Succinate buffered saline, pH 4.0 | 79.5% |

The formulation solution was filtered through a 0.2 μm filter and vialed in sterile glass vials.

### Example 16

### In vitro Stability of Lactone of Representative Tocopherol-Modified Therapeutic Drug Compounds in Presence of Human Albumin

In this example, the in vitro stability in the presence of human albumin of the lactone form of representative tocopherol-modified therapeutic drug compounds of the invention, tocopherol succinate camptothecin and tocopherol succinate 7-ethyl-10-hydroxycamptothecin, was compared to the in vitro stability of the lactone form of camptothecin.

Because the lactone (ring E) is a critical moiety for camptothecin activity and it is reported not to be stable under physiological conditions (pH=7.4), the stability of the lactone for tocopherol succinate camptothecin and tocopherol succinate 7-ethyl-10-hydroxycamptothecin was determined. The solubilization of tocopherol succinate camptothecin and tocopherol succinate 7-ethyl-10-hydroxycamptothecin in the oil-phase is thought to protect the lactone from hydrolysis and thus provide improved lactone stability in physiological conditions. To evaluate lactone stability, a saline buffered solution (10 mM, pH 7.4) containing 4% human serum albumin was incubated at 37° C. in the presence of camptothecin (dissolved in DMSO), tocopherol succinate camptothecin emulsion (prepared as described in Example 15C, referred to herein as “SN2300 emulsion”) or tocopherol succinate 7-ethyl-10-hydroxycamptothecin emulsion (prepared as described in Example 15A, referred to herein as “SN2310 emulsion”). High performance liquid chromatography with fluorescence detection was used to analyze the decrease in the concentration of the lactone form over time.

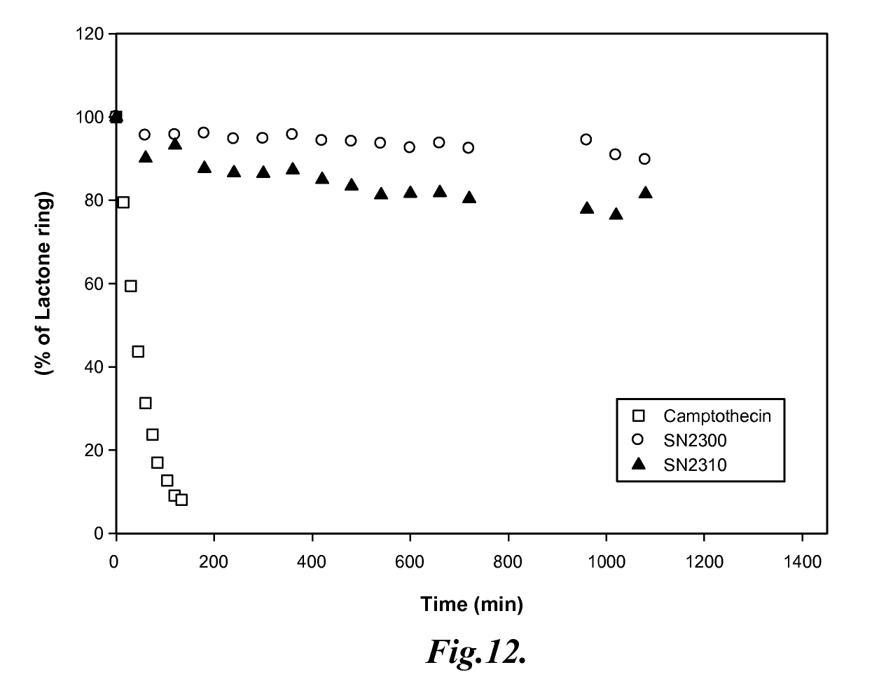

FIG. 12 illustrates the percent change in concentration of the lactone form over time for camptothecin, tocopherol succinate camptothecin (SN2300), and tocopherol succinate 7-ethyl-10-hydroxycamptothecin (SN2310), in the presence of human serum albumin. The stability of the lactone of tocopherol succinate camptothecin and tocopherol succinate 7-ethyl-10-hydroxycamptothecin is greater than that of camptothecin. This dramatic increase in the stability of the lactone may result in increased activity compared to the unmodified camptothecin parent compound.

### Example 17

### In vitro Cytotoxicity of Representative Tocopherol-Modified Therapeutic Drug Compounds

In this example, the in vitro cytotoxicity of representative tocopherol-modified therapeutic drug compounds of the invention, tocopherol succinate camptothecin and tocopherol succinate 7-ethyl-10-hydroxycamptothecin, was compared to the in vitro cytotoxicity of camptothecin, 10-hydroxycamptothecin, SN38, irinotecan, and topotecan.

The in vitro cytotoxicity, as measured by GI₅₀ (50% of growth inhibition) values, of tocopherol succinate camptothecin and tocopherol succinate 7-ethyl-10-hydroxycamptothecin was investigated and compared to the National Cancer Institute (NCl) GI₅₀ values for camptothecin, 10-hydroxycamptothecin, SN-38, irinotecan, and topotecan in the following cancer cell lines: NCI-H460 (ATCC #HTB-177) (non-small cell lung), HCT-15 (ATCC #CCL-225) (colorectal), HT-116 (ATCC #CCL-247) (colorectal), HT-29 (ATCC #HTB-38) (colorectal), MCF-7 (ATCC #HTB-22) (breast), and OVCAR-3 (ATCC #HTB-161) (ovarian).

The study was performed using emulsion formulations of tocopherol succinate 7-ethyl-10-hydroxycamptothecin (described in Example 15A) and tocopherol succinate camptothecin (described in Example 15C) diluted in the corresponding cell media. The cells were in contact with varying concentrations of the test article for a period of 48 hours. At the end of 48 hours, staining with ALAMAR BLUE was performed to determine the number of viable cells and calculate the degree of cellular growth inhibition as compared to a control group. The percent of inhibition versus concentration was fit to the Hill equation to determine concentration that produces 50% of growth inhibition (GI₅₀).

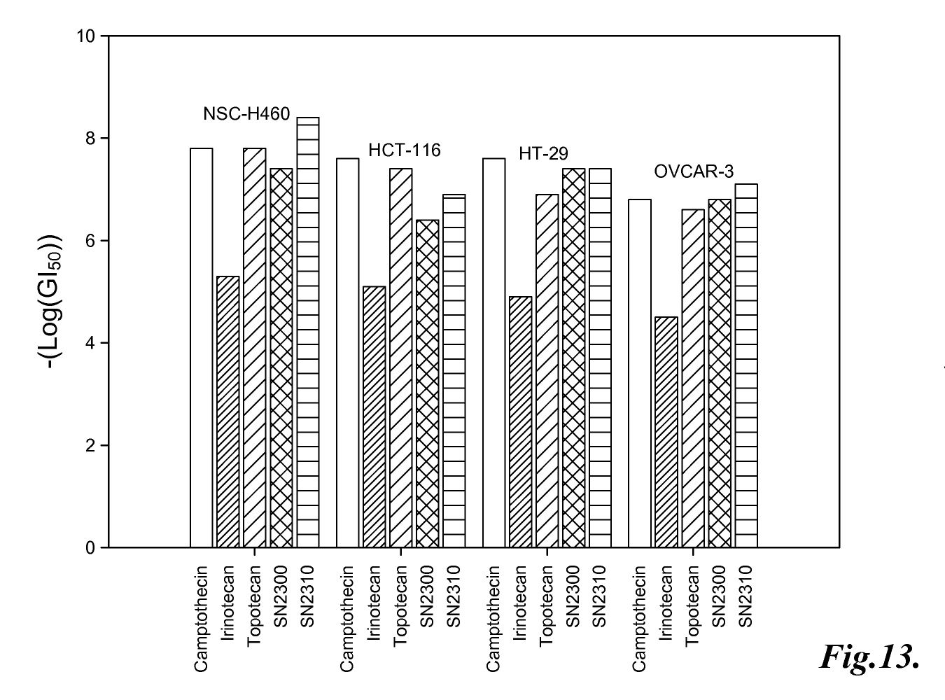

The sensitivity of the tested cell lines to tocopherol succinate camptothecin (SN2300), tocopherol succinate 7-ethyl-10-hydroxycamptothecin (SN2310), camptothecin, irinotecan, and topotecan is illustrated in Table 2 and FIG. 13.

TABLE 2
Comparative drug concentration that produce 50% cell growth inhibition (GI₅₀).

| | CPT | 10-HO- | SN38 | Irinotecan | Topotecan | VESA- | VESA- |
|---|---|---|---|---|---|---|---|
| Cell line | (NCI) | CPT | (NCI) | (NCI) | (NCI) | CPT | SN38 |
| NCI-H460 | 16 nM | 11 nM | 1.4 nM | 5.01 μM | 19.9 nM | 43 nM | 4 nM |
| (NSCLC) | | | | | | | |
| HCT-15 | 160 nM | 356 nM | 7.9 nM | 31.6 μM | 501 nM | 20 μM | 99 nM |
| (COLON) | | | | | | | |
| OVCAR-3 | 160 nM | 62 nM | 2.9 nM | 31.6 μM | 251 nM | Poor | 83 nM |
| (OVARIAN) | | | | | | activity | |
| HCT-116 | 40 nM | 27 nM | 21 nM | 7.9 μM | 39.8 nM | 449 nM | 119 nM |
| (COLON) | | | | | | | |
| HT29 | 126 nM | 112 nM | 1 nM | 12.58 μM | 125 nM | 434 nM | 91 nM |
| (COLON) | | | | | | | |
| MCF-7 | 13 nM | 10 nM | | 3.98 μM | 15.8 nM | 325 nM | |
| (BREAST) | | | | | | | |

CPT: Camptothecin;
10-HO-CPT: 10-hydroxycamptothecin;
SN38: 7-ethyl-10-hydroxycamtothecin;
VESA-CPT: tocopherol succinate camptothecin;
VESA-SN38: tocopherol succinate 7-ethyl-10-hydroxycamothecin.

The results in Table 2 illustrates that formulations of tocopherol-modified therapeutic drug compounds of the invention provide effective anti-tumor activity.

FIG. 13 is a plot of the GI₅₀ values (concentration that produces 50% growth inhibition) determined for tocopherol succinate camptothecin and tocopherol succinate 7-ethyl-10-hydroxycamptothecin in four of the tested cell lines. The values reported by the NCl in these same cancer cell lines for camptothecin, irinotecan, and topotecan is also included as comparison. A high GI₅₀ value corresponds to a low drug concentration to produce 50% inhibition. From the graph, it is clear that the compounds of the invention, tocopherol succinate camptothecin and tocopherol succinate 7-ethyl-10-hydroxycamptothecin, show a high level of cytotoxic activity similar to camptothecin.

### Example 18

### Pharmacokinetics of Representative Tocopherol-Modified Therapeutic Drug Compounds

In this example, the pharmacokinetics of representative tocopherol-modified therapeutic drug compounds of the invention, tocopherol succinate camptothecin and tocopherol succinate 7-ethyl-10-hydroxycamptothecin, was compared to camptothecin, irinotecan, and topotecan.

The pharmacokinetic profiles of tocopherol succinate camptothecin and tocopherol succinate 7-ethyl-10-hydroxycamptothecin were investigated in Sprague-Dawley rats following a bolus, intravenous administration of emulsion formulations of the drug compounds (SN2300 emulsion and SN2310 emulsion) via the lateral tail vein at a dose of approximately 14 mg of drug compound/kg of body weight. Blood samples were collected for up to 120 hours post dose via the jugular vein. The concentration of each camptothecin derivative in plasma was determined by high performance liquid chromatography (HPLC) with fluorescence detection. A noncompartmental analysis was carried out using WinNonlin (v 4.1).

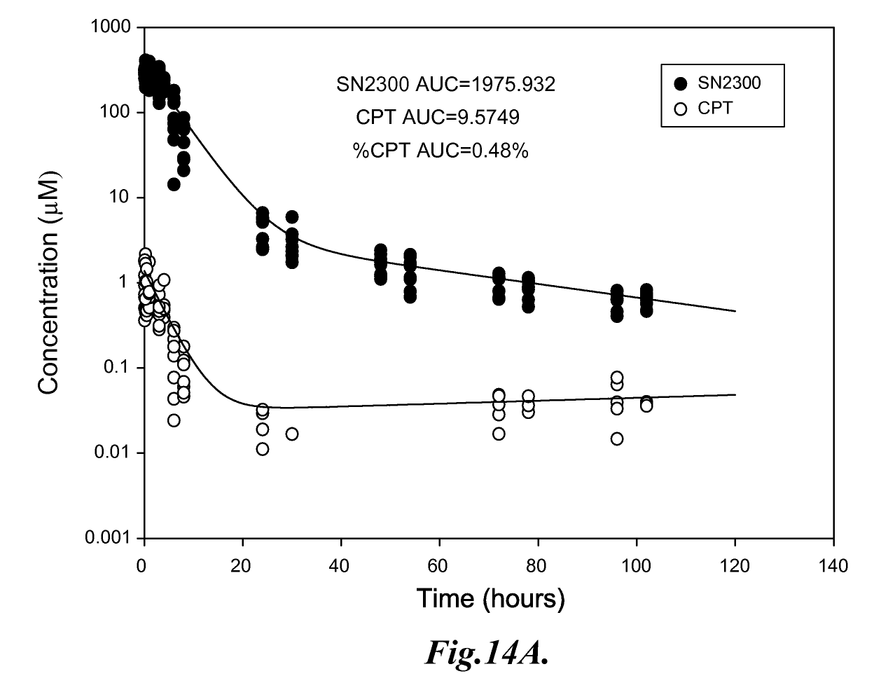

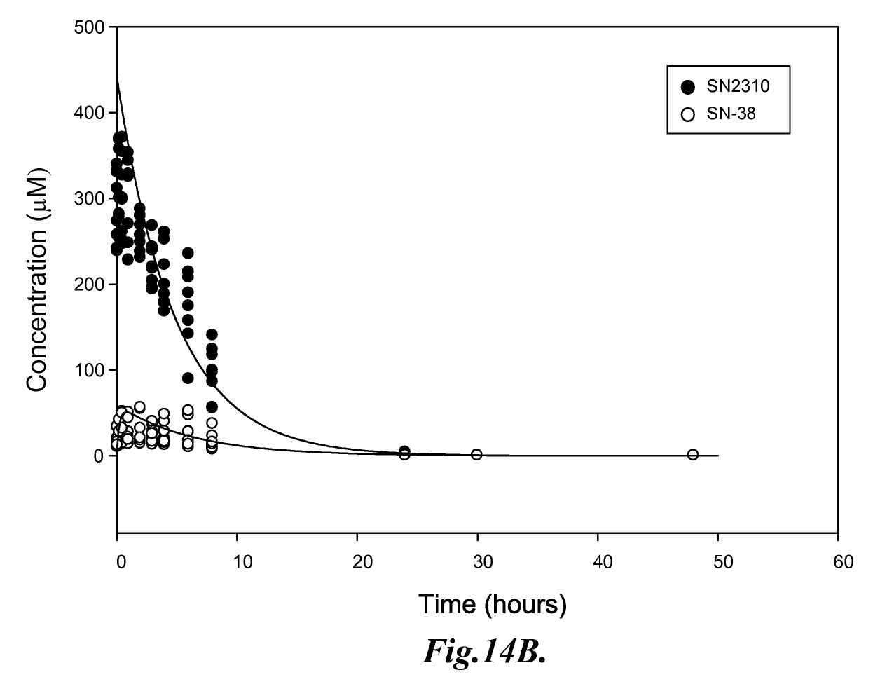

The pharmacokinetic profiles of tocopherol succinate camptothecin (SN2300) and tocopherol succinate 7-ethyl-10-hydroxycamptothecin (SN2310) are illustrated in FIGS. 14A and 14B, respectively. FIGS. 14A and 14B illustrate concentration-time values after an intravenous injection of 13.8 mg of drug compound/kg of body weight for tocopherol succinate camptothecin (SN2300 emulsion) and tocopherol succinate 7-ethyl-10-hydroxycamptothecin (SN2310 emulsion), respectively. Referring to FIGS. 14A and 14B, a prolonged plasma half-life following intravenous administration, particularly for tocopherol succinate camptothecin, is shown.

The calculated plasma elimination half-life, mean residence time, and clearance of tocopherol succinate camptothecin (SN2300), tocopherol succinate 7-ethyl-10-hydroxycamptothecin (SN2310), camptothecin, irinotecan, and topotecan is provided in Table 3.

The term “plasma elimination half-life” refers to the time necessary to reduce the drug concentration in plasma by 50% after equilibrium is reached. The term “elimination rate constant” refers to the fraction of drug eliminated per unit of time. With first-order elimination, the rate of elimination is directly proportional to the serum drug concentration. There is a linear relationship between rate of elimination and serum drug concentration. Although the amount of drug eliminated in a first-order process changes with concentration, the fraction of a drug eliminated remains constant.

The term “clearance” refers to a measure of the body's ability to eliminate drug and is a hypothetical volume of distribution of drug which is cleared per unit time (i.e., mL/min) by any pathway of drug removal. It is important to clarify that the clearance does not indicate how much drug is being removed, rather, the volume of biological fluid such as blood or plasma that would have to be completely freed of drug to account for the elimination. The term “volume of distribution” refers to a calculated volume of body fluid that would be required to dissolve the total amount of drug at the same concentration as that found in the blood. It is a proportionality constant relating the amount of drug in the body to the measured concentration in biological fluid (blood, plasma, serum).

TABLE 3
Comparative pharmacokinetic parameters following intravenous
administration in rats of tocopherol succinate camptothecin
(SN2300) and tocopherol succinate 7-ethyl-10-hydroxycamptothecin
(SN2310) compared to camptothecin, irinotecan, and topotecan.

| | t_{1/2} (hr) | MRT | Clearance |
|---|---|---|---|
| Compound | (elimination) | (hours) | (l/hr/kg) |
| SN2300 | 29.08 | 9.40 | 0.0081 |
| SN2310 | 3.49 | 5.15 | 0.0067 |
| Camptothecin^{a} | 1.7 | — | 8.81 |
| Topotecan^{a} | 1.06 | — | 3.22 |
| Irinotecan^{b} | 1.54 | 1.43 | 2.22 |

^{a}El-Gizawy SA, Hedaya MA. Cancer Chemother. Pharmacol., 43: 364-370 (1999).
^{b}Atsumi R, Okazaji O Hakusui H. Biol. Pharm. Bull., 18 (8): 1114-1119 (1995).

Table 3 illustrates that the calculated plasma elimination half-life of tocopherol succinate camptothecin (SN2300) and tocopherol succinate 7-ethyl-10-hydroxycamptothecin (SN2310) is approximately 30-times and 3-times longer than the commercially available analogs, respectively. The higher mean residence time (MRT) and lower clearance rate suggest a longer tumor exposure time to these new derivatives, which may indicate a potential for increased chemotherapeutic effect.

Through lipophilic modification of therapeutic drug compounds, the plasma elimination half-life of the parent therapeutic drug compound can be increased. The compounds of the invention, by virtue of the lipophilic moiety (e.g., tocopherol moiety), have increased plasma elimination half-life compared to the parent therapeutic drug. As illustrated above for tocopherol succinate camptothecin and tocopherol succinate 7-ethyl-10-hydroxycamptothecin, the plasma elimination half-life is significantly increased compared to the parent compounds.

### Example 19

### In vivo Anti-Tumor Activity of Representative Tocopherol-Modified Therapeutic Drug Compounds in Human Tumor Xenographs

In this example, the in vivo anti-tumor activity of representative tocopherol-modified therapeutic drug compounds of the invention, tocopherol succinate camptothecin and tocopherol succinate 7-ethyl-10-hydroxycamptothecin, was compared to the anti-tumor effect of irinotecan.

NCI-H460 Human Tumor Xenograft. Athymic mice were subcutaneously implanted with a cell suspension (10⁷ cells/mouse). When tumors reached an appropriate size, animals were randomized into groups of eight and intravenously administered the following compounds at a dose of 15 mg of drug compound/kg of body weight on a schedule of q1dx5 for two consecutive weeks:

Saline-control group

Irinotecan

SN2300 emulsion

SN2310 emulsion

HT-29 Human Tumor Xenograft. Athymic mice were subcutaneously implanted with 30-40 mg tumor fragments using 12-gauge trocar needles. A sufficient number of mice were implanted with fragments so tumors in narrow weight range (100-200 mg) were selected for the trial on staging day (SD). The animals selected with tumors in the proper size range were randomized into six groups of 10 animals and intravenously administered the following test compounds:

| | |
|---|---|
| Saline-control group | (q1dx5 for 2 weeks) |
| Irinotecan | (15 mg/kg, q1dx5 for 2 weeks) |
| SN2300 emulsion | (15 mg/kg, q1dx5 for 2 weeks) |
| SN2310 emulsion | (15 mg/kg, q1dx5 for 2 weeks) |
| SN2300 emulsion | (15 mg/kg, q3dx10) |
| SN2310 emulsion | (15 mg/kg, q3dx10) |

In both xenograph studies, animal body weights and tumors were measured twice weekly following the initiation of dosing. The tumor measurements were performed using a caliper (millimeters); the tumor volume was calculated based on the formula:
(Length×width²)/2=Volume(mm³).

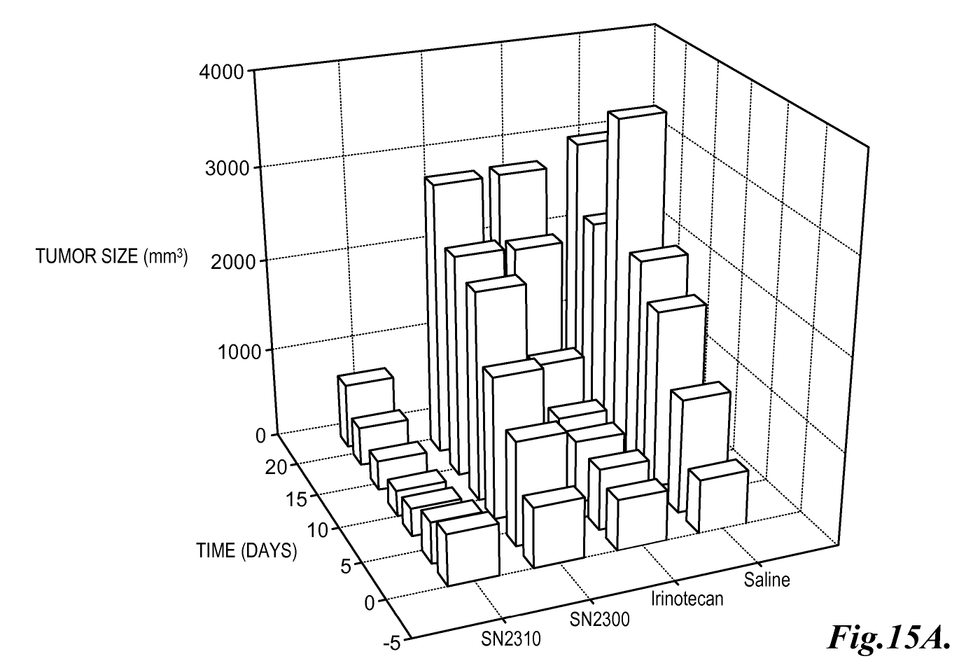

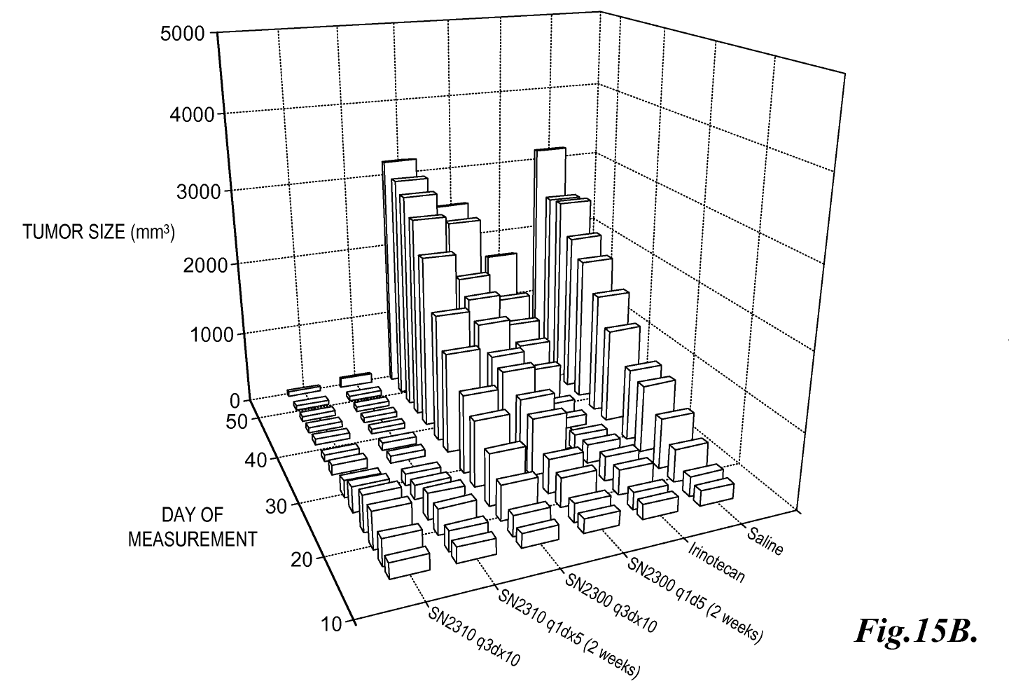

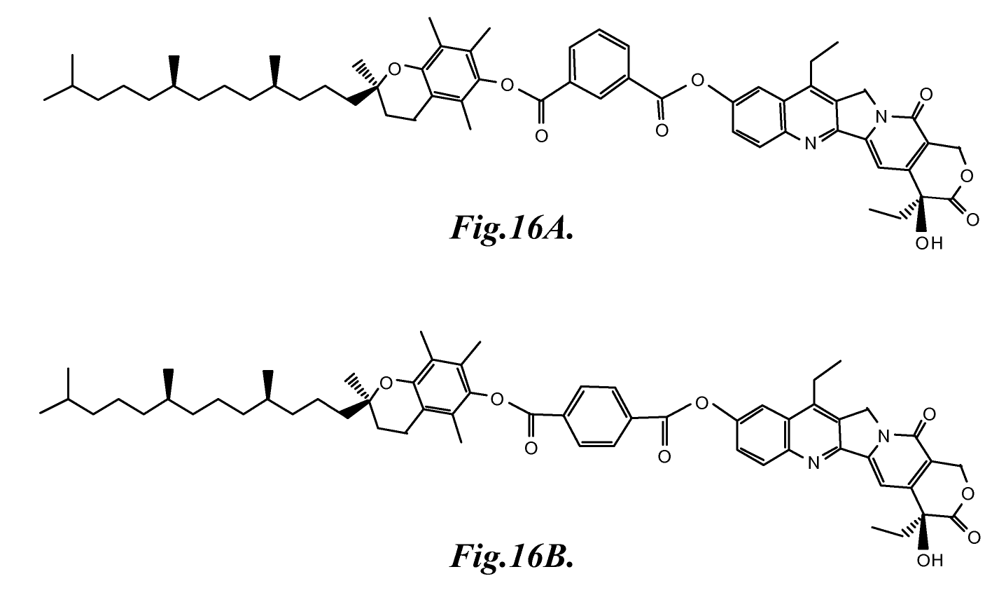

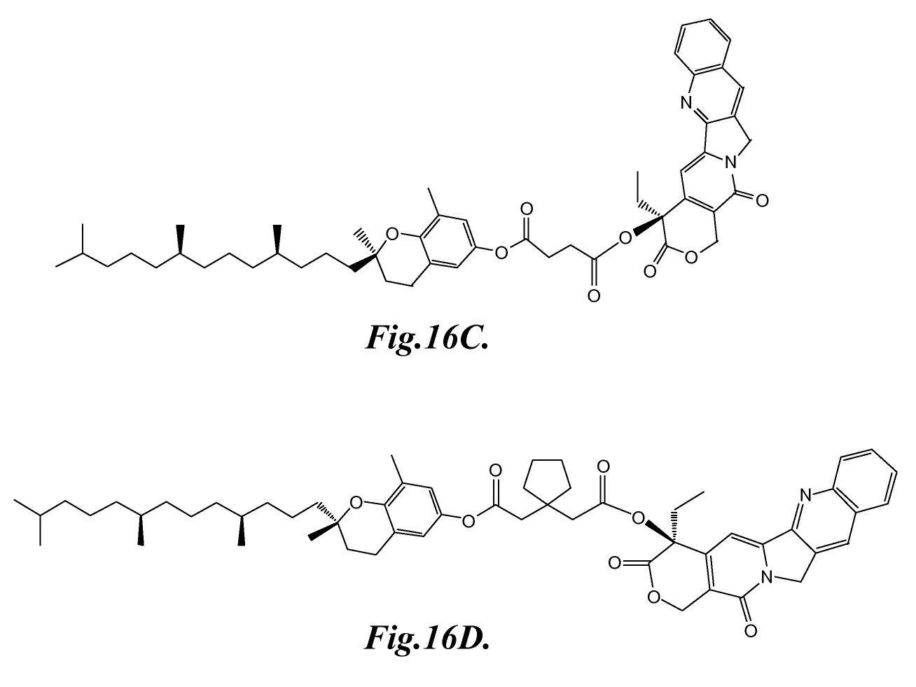

The anti-tumor effect of SN2300 and SN2310 emulsions administered to athymic mice implanted with NCI-H460 human tumor cells and HT-29 human tumor cells is graphically represented in FIGS. 15A and 15B, respectively. Although the SN2300 emulsion showed little to no anti-tumor effect in this model, the SN2310 emulsion exhibited substantial anti-tumor effect as compared to both saline control and irinotecan.

The calculated tumor response parameters for the HT-29 xenograft study is provided in Table 4. Fifty-five days after dose initiation, 30% of mice in the control group were sacrificed because of tumor size (>4000 mm³) and the median tumor size was 3136 mm³. At this same timepoint, 80% of the mice in the SN2310 (q3dx10) group presented no measurable tumor. In addition, the SN2310 (q1dx5) group had a median tumor size of 126 mm³ with 40% having no measurable tumor. At the same time, the irinotecan group showed a median tumor size of 1637 mm³. The results indicate that the administration of SN2310 produces significant anti-tumor activity.

TABLE 4
Calculated Tumor Response Parameters from the HT-29 Xenograft
Study.

| | | | | Number of |
|---|---|---|---|---|
| | | Tumor | Tumor | animals with |
| | | Growth | Growth | no measurable |
| | | Delay (T-C) | Inhibition | tumor on |
| Group | Schedule | (days) | (% T/C) | Day 55 |
| Saline | q1dx5 (2 weeks) | — | — | 0/10 |
| Irinotecan | q1dx5 (2 weeks) | 14 | 52 | 0/10 |
| SN2300 | q1dx5 (2 weeks) | 8.5 | 79 | 0/10 |
| SN2300 | q3dx10 | 0 | 102 | 0/10 |
| SN2310 | q1dx5 (2 weeks) | >28* | 4 | 4/10 |
| SN2310 | q3dx10 | >28* | 2 | 8/10 |

*90% of tumors in this group had still not reached maximum pre-determined size.
T-C = Median Time for Treatment group (T) and Control group (C) to reach a predetermined size.
% T/C = (Treated median tumor weight)/(Control median tumor weight) × 100 (at Day 55).

### Example 20

### The Preparation of a Representative Tocopherol-Modified Camptothecin Compound: d-α-Tocopherolglutarate-7-ethyl-10-hydroxycamptothecin

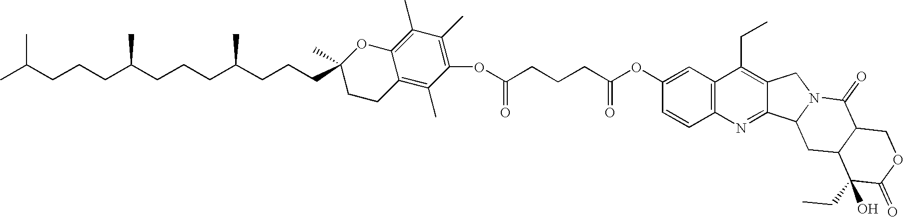

Preparation of d-α-tocopherol glutaric acid. A mixture of 8.60 g (0.02 mol) d-α-tocopherol, 1.14 g (0.01 mol) glutaric anhydride, 1.33 g (0.01 mol) aluminum chloride, and 200 mL of cyclohexane in a 500 mL flask was heated for 30 minutes at refluxing temperature under nitrogen. Thin layer chromatography showed the reaction was complete. After the mixture was cooled down to room temperature, it was washed with 0.1 M hydrochloric acid (3×100 mL), and then dried with anhydrous magnesium sulfate. The magnesium sulfate was removed by filtration, and the mixture was concentrated to 10 mL. The crude product was purified by column chromatography on silica gel to provide 2.82 g (51.84%) of d-α-tocopherol glutaric acid.

Preparation of d-α-tocopherolglutarate-7-ethyl-10-hydroxycamptothecin. A mixture of 1.63 g (0.003 mol) of d-α-tocopherol glutaric acid, prepared as above, in 50 mL of toluene and 0.71 g (0.006 mol) of thionyl chloride was stirred overnight at room temperature under nitrogen. The toluene and excess thionyl chloride were removed under reduced pressure. To the residue was added 10 mL of methylene chloride to form Solution A. In another 100 mL flask was added 0.588 g (0.0015 mol) of 7-ethyl-10-hydroxycamptothecin, 25 mL of anhydrous N,N-dimethylacetamide, and 0.303 g (417 μL, 0.003 mol) of triethylamine to form Solution B. The mixture was stirred until the solution became clear. Solution A was added to Solution B. The resulting mixture was stirred overnight at room temperature under nitrogen. To the reaction mixture was added 150 mL of ethyl acetate. The mixture was washed with NaCl aqueous solution (3×100 mL). The ethyl acetate solution was dried with anhydrous magnesium sulfate for 2 hours. The magnesium sulfate was removed by filtration and the filtrate was collected. The filtrate was concentrated to about 10 mL. The crude product was purified by column chromatography on silica gel to provide 0.686 g (49.8%) of d-α-Tocopherolglutarate-7-ethyl-10-hydroxycamptothecin.

IRνₘₐₓ^{KBr} cm⁻¹: 3254.26, 2925.35, 1738.34, 1660.65, 1597.92, 1508.77, 1455.16, 1414.10, 1376.72, 1281.68, 1226.51, 1211.78, 1162.89, 1126.98, 1106.32, 1064.03, 1012.10, 917.21, 829.66, 758.88, 723.24, 665.87.

MS (Positive ESI): m/z 919.8 (M+H)⁺.

¹H NMR (300 MHz, CDCl₃): δ 8.279-8.248 (d, J=9.3 Hz, 1H), 7.868-7.860 (d, J=2.4 Hz, 1H), 7.668 (s, 1H), 7.604-7.566 (dd, J₁=9.0 Hz, J₂=2.4 Hz, 1H), 5.805-5.750 (d, J=16.5 Hz, 1H), 5.357-5.303 (d, J=16.2 Hz, 1H), 5.283 (s, 2H), 3.771 (s, 1H), 3.206-3.128 (q, 2H), 2.900-2.817 (m, 4H), 2.612 (t, 2H), 2.355-2.163 (m, 2H), 2.112 (s, 3H), 2.054 (s, 3H), 2.013 (s, 3H), 1.961-1.866 (m, 2H), 1.842-1.743 (m, 2H), 1.580-1.034 (m, 30H), 0.889-0.845 (m, 12H).

Elemental anal. Calcd. for C₅₆H₇₄N₂O₉: C, 73.17; H, 8.11; N, 3.05.

Found: C, 72.91; H, 8.20; N, 3.01.

### Example 21

### The Preparation of a Representative Tocopherol-Modified Camptothecin Compound: d-α-Tocopherol-3-methylglutarate-7-ethyl-10-hydroxycamptothecin

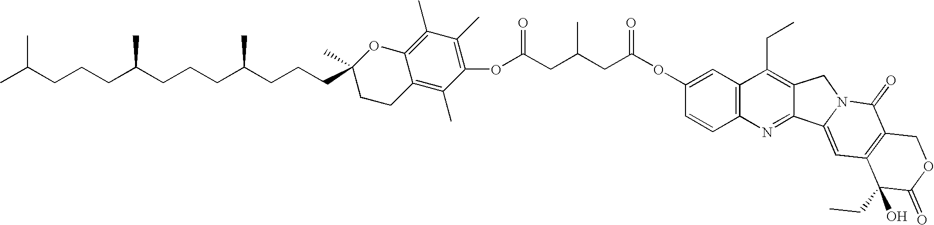

Preparation of d-α-tocopherol-3-methylglutaric acid. In a 300 mL flask, a mixture of 4.30 g (0.01 mol) of d-α-tocopherol, 1.28 g (0.01 mol) of 3-methylglutaric anhydride, 1.33 g (0.01 mol) of aluminum chloride, and 150 mL of cyclohexane was stirred for 2 hours at refluxing temperature under nitrogen. After the mixture was cooled down to room temperature, the mixture was washed with 0.1 M HCl (3×100 mL), and dried with anhydrous magnesium sulfate for 2 hours. The crude product was purified by column chromatography on silica gel to provide 3.41 g (61.0%) of d-α-tocopherol-3-methylglutaric acid.

Preparation of d-α-tocopherol-3-methylglutarate-7-ethyl-10-hydroxycamptothecin. A mixture of 1.67 g (3 mmol) d-α-tocopherol-3-methylglutaric acid, prepared as above, 0.48 g (4 mmol) of thionyl chloride, and 50 mL of toluene was stirred overnight at room temperature under nitrogen. The solvent and excess thionyl chloride were removed under reduced pressure at 50° C. The residue was collected and dissolved in 10 mL of chloroform to form Solution A. In another 100 mL flask, 0.392 g (1 mmol) of 7-ethyl-10-hydroxycamptothecin was dissolved in 20 mL of anhydrous N,N-dimethylacetamide to form Solution B. Solution A was added to Solution B. Then, 0.303 g (3 mmol) of triethylamine was added to the mixture. The resulting mixture was stirred overnight at room temperature under nitrogen. Thin layer chromatography showed that the reaction was complete. To the reaction mixture was added 150 mL of ethyl acetate. The mixture was washed with NaCl aqueous solution (3×100 mL), and dried with anhydrous magnesium sulfate for 2 hours. The magnesium sulfate was removed by filtration. The filtrate was evaporated until dryness under reduced pressure. The crude product was purified by column chromatography on silica gel to provide 0.374 g (40.08%) of d-α-tocopherol-3-methylglutarate 7-ethyl-10-hydroxycamptothecin.

IRνₘₐₓ^{KBr} cm⁻¹: 3357.35, 2925.41, 2868.27, 1749.42, 1659.96, 1606.57, 1557.77, 1510.73, 1460.38, 1414.59, 1376.07, 1329.60, 1246.22, 1226.08, 1156.53, 1107.25, 1079.59, 1032.23, 997.70, 980.59, 916.64, 866.44, 836.62, 808.44, 723.43, 696.46, 669.22.

¹H NMR (300 MHz, CDCl₃): δ 8.260-8.230 (d, J=9.0 Hz, 1H), 7.841-7.833 (d, J=2.4 Hz, 1H), 7.654 (s, 1H), 7.587-7.548 (dd, J₁=9.3 Hz, J₂=2.7 Hz, 1H), 5.789-5.735 (d, J=16.2 Hz, 1H), 5.342-5.287 (d, J=16.5 Hz, 1H), 5.265 (s, 2H), 3.768 (s, 1H), 3.180-3.103 (q, J=7.8 Hz, 2H), 2.912-2.668 (m, 5H), 2.616-2.571 (t, J=6.7 Hz, 2H), 2.094 (s, 3H), 2.040 (s, 3H), 1.998 (s, 3H), 1.947-1.853 (m, 2H), 1.822-1.726 (m, 2H), 1.590-1.019 (m, 33H), 0.875-0.831 (m, 12H).

Elemental anal. Calcd. for C₅₇H₇₆N₂O₉: C, 73.36; H, 8.21; N, 3.00.

Found: C, 73.05; H, 8.22; N, 2.96.

### Example 22

### The Preparation of a Representative Tocopherol-Modified Camptothecin Compound: d-α-Tocopherol-3,3-tetramethyleneglutarate-7-ethyl-10-hydroxycamptothecin

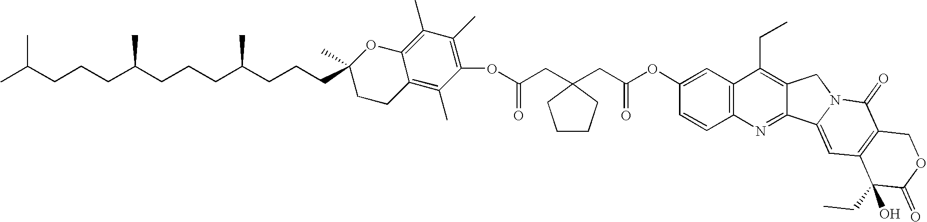

Preparation of d-α-tocopherol-3,3-tetramethyleneglutaric acid. A 300 mL flask was charged with 4.30 g (10 mmol) of d-α-tocopherol, 2.52 g (15 mmol) of 3,3-tetramethyleneglutaric anhydride, 1.33 g (10 mmol) of aluminum chloride and 150 mL of cyclohexane. The mixture was stirred for 30 minutes at refluxing temperature under nitrogen. Thin layer chromatography showed the reaction was complete. After the reaction was cooled down to room temperature, the mixture was washed with 0.1 M hydrochloric acid (3×100 mL), and then dried with anhydrous magnesium sulfate. The magnesium sulfate was removed by filtration. The filtrate was evaporated until dryness under reduced pressure. The crude product was purified by column chromatography on silica gel to provide 3.72 g (62.1%) of d-α-tocopherol-3,3-tetramethyleneglutaric acid.

Preparation of d-α-tocopherol-3,3-tetramethyleneglutarate-7-ethyl-10-hydroxycamptothecin. A 100 mL flask was charged with 2.75 g (4.5 mmol) of d-α-tocopherol-3,3-tetramethyleneglutaric acid, prepared as above, 50 mL of toluene, 1.61 g (15 mmol) of thionyl chloride. The mixture was stirred overnight at room temperature under nitrogen. The solvent and excess thionyl chloride were removed under reduced pressure. The residue was dissolved in 10 mL of dichloromethane to form Solution A. To another 50 mL flask was added 0.588 g (1.5 mmol) of 7-ethyl-10-hydroxycamptothecin and 20 mL of anhydrous N,N-dimethylacetamide to form Solution B. Solution A and 0.455 g (626 μL) of triethylamine was added to Solution B. The resulting mixture was stirred overnight at room temperature under nitrogen. The reaction mixture was concentrated under reduced pressure. The crude product was purified by column chromatography to provide 0.98 g (67.1%) of d-α-tocopherol-3,3-tetramethyleneglutarate-7-ethyl-10-hydroxycamptothecin.

¹H NMR (300 MHz, CDCl₃): δ 8.251-8.220 (d, J=9.3 Hz, 1H), 7.803-7.794 (d, J=2.7 Hz, 1H), 7.653 (s, 1H), 7.577-7.539 (dd, J₁=9.0 Hz, J₂=2.4 Hz, 1H), 5.786-5.732 (d, J=16.2 Hz, 1H), 5.339-5.284 (d, J=16.5 Hz, 1H), 5.260 (s, 2H), 3.792 (s, 1H), 3.168-3.092 (q, J=7.8 Hz, 2H), 3.054 (s, 2H), 3.017 (s, 2H), 2.607-2.564 (t, J=6.5 Hz, 2H), 2.086 (s, 3H), 2.037 (s, 3H), 1.995 (s, 3H), 1.945-1.718 (m, 12H), 1.598-1.016 (m, 30H), 0.872-0.829 (m, 12H).

Elemental anal. Calcd. for C₆₀H₈₀N₂O₉: C, 74.04; H, 8.28; N, 2.88.

Found: C, 73.93; H, 8.33; N, 2.80.

### Example 23

### The Preparation of a Representative Tocopherol-Modified Camptothecin Compound: d-α-Tocopheroladipate-7-ethyl-10-hydroxycamptothecin

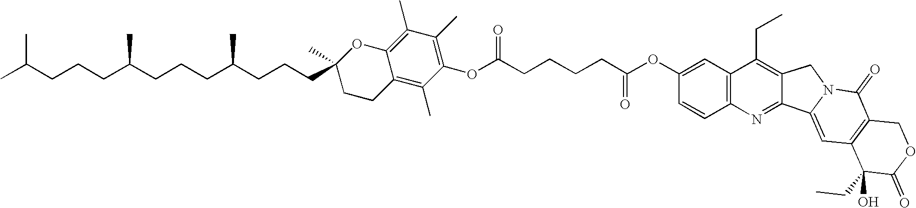

Preparation of d-α-tocopherol adipic acid. A mixture of 4.30 g (10 mmol) of d-α-tocopherol, 7.31 g (50 mmol) of adipic acid, 4.88 g (40 mmol) of 4-diemthylaminopyrindine, 5.1 g (20 mmol) of 2-chloro-1-methylpyridinium iodide, and 100 mL of N,N-dimethylformamide was stirred for 24 hours at room temperature. To the mixture was added 150 mL of ethyl acetate. The mixture was washed with saturated NaCl aqueous solution (3×100 mL), and dried with anhydrous magnesium sulfate. The magnesium sulfate was removed by filtration. The filtrate was then concentrated to about 10 mL. The crude product was purified by column chromatography on silica gel to provide 3.357 g (60.6%) of d-α-tocopherol adipic acid.

Preparation of d-α-tocopheroladipate-7-ethyl-10-hydroxycamptothecin. In a 200 mL flask, a mixture of 0.80 g (1.43 mmol) of d-α-tocopherol adipic acid, prepared as above), 1.30 g (800 μL, 10.9 mmol) of thionyl chloride, 100 mL of N,N-dimethylformamide, and 100 mL of toluene was stirred for 4 hours at room temperature under nitrogen. The solvent and excess thionyl chloride were removed under reduced pressure. To the residue was added 0.196 g (0.5 mmol) of 7-ethyl-10-hydroxycamptothecin, 30 mL of N,N-dimethylacetamide, and 0.101 g (139 μL, 1 mmol) of triethylamine. The resulting mixture was stirred overnight at room temperature under nitrogen. After reaction was complete, 200 mL of ethyl acetate was added into the reaction mixture. The resulting mixture was washed with saturated sodium chloride aqueous solution (3×100 mL), and dried with anhydrous magnesium sulfate for 2 hrs. The magnesium sulfate was removed. The filtrate was concentrated to about 10 mL. The crude product was purified by column chromatography on silica gel to provide 0.385 g (82.6%) of d-α-tocopheroladipate-7-ethyl-10-hydroxycamptothecin

IRνₘₐₓ^{KBr} cm⁻¹: 3241.68, 2925.15, 2868.15, 1739.70, 1659.97, 1597.26, 1570.51, 1510.21, 1462.72, 1414.74, 1376.86, 1331.19, 1282.59, 1248.45, 1227.54, 1211.57, 1164.48, 1127.82, 1106.03, 1088.92, 1064.71, 1040.37, 1013.07, 971.98, 922.66, 866.05, 848.96, 830.46, 810.26, 759.10, 723.68, 664.33.

MS (Positive ESI): m/z 934.0 (M+H)⁺.

¹H NMR (300 MHz, CDCl₃): δ 8.275-8.245 (d, J=9.0 Hz, 1H), 7.866-7.858 (d, J=2.4 Hz, 1H), 7.669 (s, 1H), 7.600-7.562 (dd, J₁=9.3 Hz, J₂=2.4 Hz, 1H), 5.806-5.752 (d, J=16.2 Hz, 1H), 5.359-5.305 (d, J=16.2 Hz, 1H), 5.284 (s, 2H), 3.780 (s, 1H), 3.202-3.094 (m, 2H), 2.774-2.734 (m, 2H), 2.670-2.582 (m, 2H), 2.471-2.358 (m, 2H), 2.107-1.974 (m, 13H), 1.939-1.783 (m, 4H), 1.580-1.035 (m, 30H), 0.890-0.846 (m, 12H).

### Example 24

### The Preparation of a Representative Tocopherol-Modified Camptothecin Compound: d-α-Tocopherolsuberate-7-ethyl-10-hydroxycamptothecin

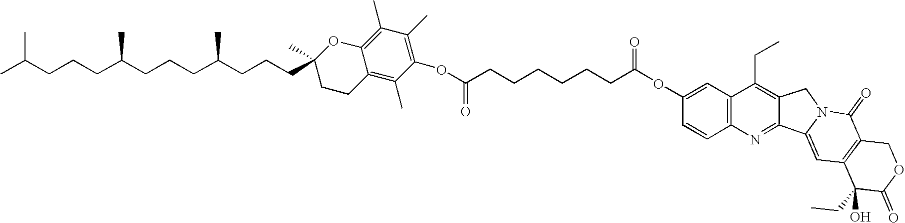

Preparation of d-α-tocopherol suberic acid. A mixture of 4.30 g (0.01 mol) of d-α-tocopherol, 8.70 g (0.05 mol) of suberic acid, 2.44 g (0.02 mol) of 4-dimethylaminopyridine, 2.55 g (0.01 mol) of 2-chloro-1-methylpyridinium iodide, and 100 mL of N,N-dimethylformamide was stirred overnight at room temperature under nitrogen. To the mixture was added 150 mL of ethyl acetate. The mixture was washed with NaCl aqueous solution (3×100 mL), and dried with anhydrous magnesium sulfate. The crude product was purified by column chromatography on silica gel to provide 3.618 g (61.65%) of d-α-tocopherol suberic acid.

Preparation of d-α-tocopherolsuberate-7-ethyl-10-hydroxycamptothecin. A mixture of 1.17 g (2 mmol) of d-α-tocopherol suberic acid, prepared as above, 0.47 g (4 mmol) of thionyl chloride, 100 μL of N,N-dimethylformamide, and 100 mL of toluene was stirred for 4 hours at room temperature under nitrogen. The toluene and excess thionyl chloride were removed by reduced pressure. The residue was collected and dissolved in 5 mL of chloroform to form Solution A. In another 100 mL flask, 0.392 g (1 mmol) of 7-ethyl-10-hydroxycamptothecin was dissolved in 30 mL of anhydrous N,N-dimethylacetamide to form Solution B. Solution A and 0.202 g (278 μL, 2 mmol) of triethylamine was added to Solution B. The resulting mixture was stirred at room temperature under nitrogen until the reaction was complete. The crude product was purified by column chromatography on silica gel to provide 0.60 g (62.42%) of d-α-tocopherolsuberate-7-ethyl-10-hydroxycamptothecin.

IRνₘₐₓ^{KBr} cm⁻¹: 3260.58, 2925.75, 2866.57, 1752.28, 1660.47, 1597.94, 1509.43, 1460.95, 1414.75, 1377.23, 1281.40, 1226.75, 1211.14, 1160.38, 1107.01, 1063.86, 1038.87, 1012.83, 970.08, 918.86, 885.17, 848.11, 830.67, 809.89, 758.86, 723.95, 665.72.

MS (Positive ESI): m/z 961.8 (M+H)⁺.

¹H NMR (300 MHz, CDCl₃): δ 8.273-8.242 (d, J=9.3 Hz, 1H), 7.851-7.843 (d, J=2.4 Hz, 1H), 7.667 (s, 1H), 7.593-7.555 (dd, J₁=9.0 Hz, J₂=2.4 Hz, 1H), 5.806-5.751 (d, J=16.5 Hz, 1H), 5.358-5.304 (d, J=16.2 Hz, 1 Hz), 5.281 (s, 2H), 3.766 (s, 1H), 3.210-3.136 (q, J=7.4 Hz, 2H), 2.730-2.576 (m, 6H), 2.102 (s, 3H), 2.031 (s, 3H), 1.988 (s, 3H), 1.963-1.752 (m, 8H), 1.588-1.035 (m, 34H), 0.890-0.846 (m, 12H).

### Example 25

### The Preparation of a Representative Tocopherol-Modified Camptothecin Compound: d-α-Tocopherolsebacate-7-ethyl-10-hydroxycamptothecin

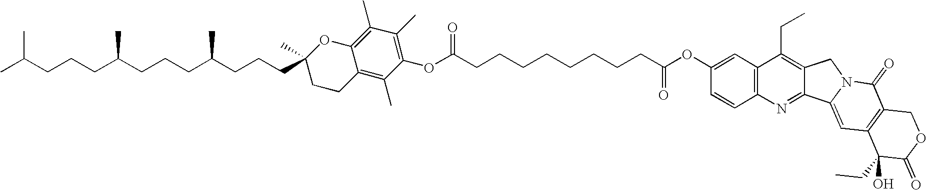

Preparation of d-α-tocopherol sebacic acid. In a 200 mL flask, a mixture of 4.3 g (10 mmol) of d-α-tocopherol, 10.0 g (49.4 mmol) of sebacic acid, 5.0 g (40.9 mmol) of 4-dimethylaminopyridine, 5.0 g (19.6 mmol) of 2-chloro-1-methylpyridinium iodide, and 100 mL of N,N-dimethylformamide was stirred for 4 hours at room temperature under nitrogen. To the mixture was added 200 mL of diethyl ether. The mixture was washed with saturated NaCl aqueous solution (3×100 mL), and then dried with anhydrous magnesium sulfate for 2 hours. The magnesium sulfate was removed by filtration. The diethyl ether was evaporated under reduced pressure. The crude product was purified by column chromatography on silica gel to provide 2.8 g (46.12%) of d-α-tocopherol sebacic acid.

Preparation of d-α-tocopherolsebacate-7-ethyl-10-hydroxycamptothecin. In a 200 mL flask, a mixture of 0.80 g (1.3 mmol) of d-α-tocopherol sebacic acid, prepared as above, 1.31 g (800 μL, 11 mmol) of thionyl chloride, 100 μL of N,N-dimethylformamide, 100 mL of toluene was stirred for 4 hours at room temperature under nitrogen. The toluene and excess thionyl chloride were removed under reduced pressure. To the residue was added 0.196 g (0.5 mmol) of 7-ethyl-10-hydroxycamptothecin, 30 mL of N,N-dimethylacetamide, and 0.101 g (1 mmol) of triethylamine. The mixture was stirred overnight at room temperature under nitrogen. To the mixture was added 200 mL of ethyl acetate. The resulting mixture was washed with saturated NaCl aqueous solution (3×100 mL), and then dried with anhydrous magnesium sulfate for 2 hours. The magnesium sulfate was removed by filtration. The filtrate was concentrated to obtain the desired product in its crude form. The crude product was purified by column chromatography to provide 0.146 g (29.6%) of d-α-tocopherolsebacate-7-ethyl-10-hydroxycamptothecin.

IRνₘₐₓ^{KBr} cm⁻¹: 3254.93, 2925.63, 2867.29, 1742.11, 1660.51, 1598.06, 1508.71, 1461.86, 1414.81, 1377.08, 1313.27, 1282.78, 1226.92, 1212.12, 1175.90, 1146.56, 1130.50, 1106.88, 1064.41, 1040.06, 1012.83, 973.65, 922.26, 863.91, 849.17, 831.36, 809.90, 759.09, 723.45, 664.84.

MS (Positive ESI): m/z 989.8 (M+H)⁺.

¹H NMR (300 MHz, CDCl₃): 8.272-8.241 (d, J=9.3 Hz, 1H), 7.848-7.840 (d, J=2.4 Hz, 1H), 7.666 (s, 1H), 7.592-7.553 (dd, J₁=9.0 Hz, J₂=2.4 Hz, 1H), 5.806 5.751 (d, J=16.5 Hz, 1H), 5.358-5.304 (d, J=16.2 Hz, 1H), 5.281 (s, 2H), 3.759 (s, 1H), 3.301-3.173 (q, J=7.8 Hz, 2H), 2.712-2.570 (m, 6H), 2.099 (s, 3H), 2.025 (s, 3H), 1.982 (s, 3H), 1.938-1.726 (m, 46), 0.867 (m, 12H).

### Example 26

### The Preparation of a Representative Tocopherol-Modified Camptothecin Compound: d-α-Tocopheroldodecanedioate 7-ethyl-10-hydroxycamptothecin

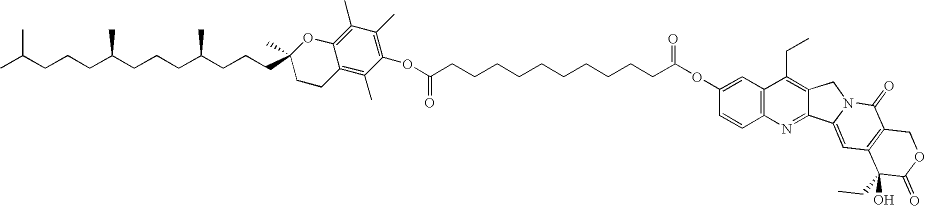

Preparation of d-α-tocopherol dodecanedioic acid. In a 200 mL flask, a mixture of 4.3 g (10 mmol) of d-α-tocopherol, 11.5 g (50 mmol) of dodecanedioic acid, 5.0 g (41 mmol) of 4-dimethylaminopyridine, 5.0 g (20 mmol) of 2-chloro-1-methylpyridinium iodide, and 100 mL of N,N-dimethylformamide was stirred overnight at room temperature under nitrogen. After the reaction was complete, 200 mL of diethyl ether was added into the reaction mixture. The resulting mixture was washed with saturated NaCl aqueous solution three times (3×100 mL), and dried with anhydrous magnesium sulfate for 2 hours. The magnesium sulfate was removed by filtration. The filtrate was collected and evaporated until dryness. The crude product was then purified by column chromatography to provide 3.211 g (51.4%) of d-α-tocopherol dodecanedioic acid.

Preparation of d-α-tocopheroldodecanedioate-7-ethyl-10-hydroxycamptothecin. In a 200 mL flask, a mixture of 0.80 g (1.28 mmol) of d-α-tocopherol dodecanedioic acid, prepared as above, 1.31 g (800 μL, 11 mmol) of thionyl chloride, 100 μL of N,N-dimethylformamide, and 100 mL of toluene was stirred for 4 hours at room temperature under nitrogen. The toluene and excess thionyl chloride were removed under reduced pressure. To the residue was added 0.392 g (1 mmol) of 7-ethyl-10-hydroxycamptothecin, 30 mL of N,N-dimethylacetamide, and 0.101 g (1 mmol) of triethylamine. The mixture was stirred overnight at room temperature under nitrogen. To the mixture was added 200 mL of ethyl acetate. The resulting mixture was washed with saturated NaCl aqueous solution three times (3×100 mL), and dried with anhydrous magnesium sulfate for 2 hours. The magnesium sulfate was removed by filtration. The filtrate was evaporated under reduced pressure until dryness. The crude product was purified by column chromatography to provide 0.576 g (53.8%) of d-α-tocopherol dodecanedioate-7-ethyl-10-hydroxycamptothecin.

IRνₘₐₓ^{KBr} cm⁻¹: 3257.95, 2924.87, 2856.07, 1741.47, 1710.38, 1659.32, 1596.88, 1508.55, 1451.11, 1414.11, 1376.32, 1312.05, 1282.05, 1247.89, 1227.03, 1211.16, 1163.99, 1144.57, 1105.34, 1064.75, 1013.35, 922.19, 849.14, 829.21, 785.51, 758.69, 735.73, 723.41, 664.16.

¹H NMR (300 MHz, CDCl₃): δ 8.276-8.246 (d, J=9.0 Hz, 1H), 7.848-7.840 (d, J=2.4 Hz, 1H), 7.637 (s, 1H), 7.593-7.554 (dd, J₁=9.3 Hz, J₂=2.7 Hz, 1H), 5.807-5.752 (d, J=16.5 Hz, 1H), 5.360-5.305 (d, J=16.5 Hz, 1H), 5.287 (s, 2H), 3.777 (s, 1H), 3.213-3.138 (q, J=7.5 Hz, 2H), 2.707-2.657 (t, J=7.2 Hz, 2H), 2.625-2.575 (m, 4H), 2.394-2.344 (t, J=7.2 Hz, 2H), 2.099 (s, 3H), 2.022 (s, 3H), 1.979 (s, 3H), 1.939-1.035 (m, 50H), 0.891-0.846 (m, 12H).

### Example 27

### The Preparation of a Representative Tocopherol-Modified Camptothecin Compound: d-α-Tocopherol tetradecanedioate 7-ethyl-10-hydroxycamptothecin

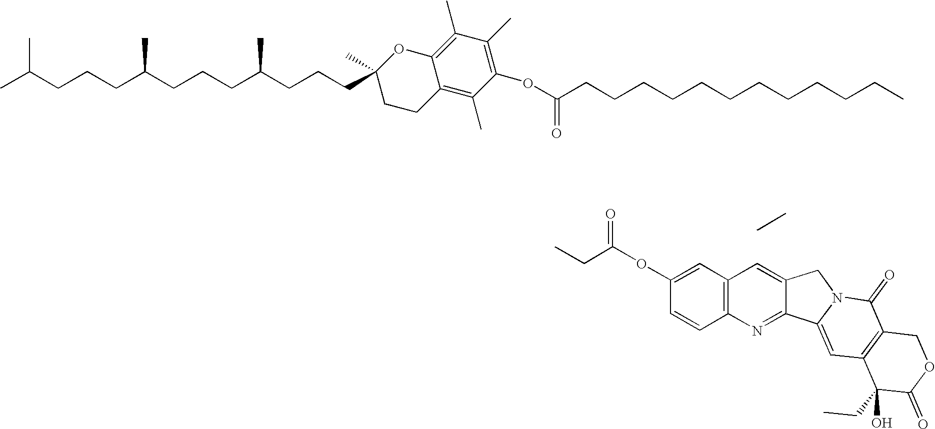

Preparation of d-α-tocopherol tetradecanedioic acid. In a 200 mL flask, a mixture of 2.15 g (5 mmol) of d-α-tocopherol, 6.25 g (24.2 mmol) of 1,12-dodecanedicarboxylic acid, 2.5 g (20.5 mmol) of 4-dimethylaminopyridine, 2.50 g (9.8 mmol) of 2-chloro-1-methylpyridinium iodide, and 100 mL of N,N-dimethylformamide was stirred overnight at room temperature under nitrogen. After reaction was complete, 200 mL of diethyl ether was added into the reaction mixture. The resulting mixture was washed three times with saturated NaCl aqueous solution (3×100 mL), and then dried with anhydrous magnesium sulfate for 2 hours. The magnesium sulfate was removed by filtration. The filtrate was evaporated under reduced pressure until dryness. The crude product was purified by column chromatography to provide 2.093 g (62.5%) of d-α-tocopherol tetradecanedioic acid.

Preparation of d-α-tocopheroltetradecanedioate-7-ethyl-10-hydroxycamptothecin. In a 200 mL flask, a mixture of 0.80 g (1.2 mmol) of d-α-tocopherol tetradecanedioic acid, prepared as above, 1.31 g (800 μL, 11 mmol) of thionyl chloride, 100 mL of N,N-dimethylformamide, and 100 ml of toluene was stirred for 4 hours at room temperature under nitrogen. The toluene and excess thionyl chloride were removed under reduced pressure. To the residue was added 0.196 g (0.5 mmol) of 7-ethyl-10-hydrorxycamptothecin, 0.101 g (1 mmol) of triethylamine, and 30 mL of N,N-dimethylacetamide. The mixture was stirred overnight at room temperature under nitrogen. To the mixture was added 200 mL of ethyl acetate. The resulting mixture was washed with saturated NaCl aqueous solution three times (3×100 mL) and dried with anhydrous magnesium sulfate for 2 hours. The magnesium sulfate was removed by filtration. The filtrate was evaporated under reduced pressure until dryness. The crude product was purified by column chromatography to provide 48.5 mg (8.8%) of d-α-tocopheroltetradecanedioate-7-ethyl-10-hydroxycamptothecin.

IRνₘₐₓ^{KBr} cm⁻¹: 3256.68, 2924.59, 2853.33, 1747.86, 1711.13, 1659.53, 1610.09, 1556.69, 1512.37, 1460.52, 1414.52, 1376.68, 1329.93, 1299.80, 1272.15, 1234.63, 1214.60, 1151.82, 1099.14, 1032.41, 998.68, 918.18, 839.14, 812.00, 724.49, 670.77.

### Example 28

### The Preparation of a Representative Tocopherol-Modified Camptothecin Compound: dl-α-Tocopherol-isophthalate-camptothecin

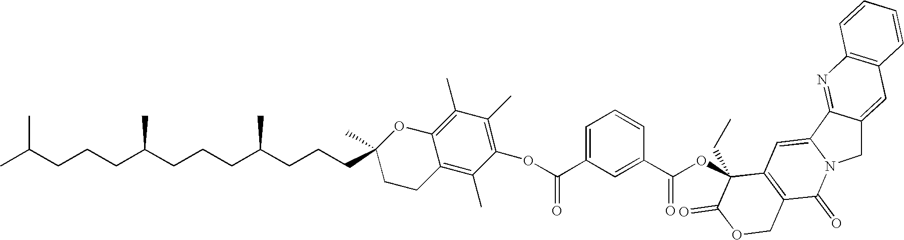

Preparation of mono-dl-α-tocopherol isophthalate. A 100 mL flask was charged with 4.30 g (0.01 mol) of dl-α-tocopherol, 5.0 g (0.03 mol) of isophthalic acid, 3.83 g (0.015 mol) of 2-chloro-1-methylpyridinium iodide, 3.66 g (0.03 mol) of 4 dimethylaminopyridine, and 50 mL of N,N-dimethylacetamide. The mixture was stirred for 4 hours at about 50° C. under nitrogen, and then allowed to be stirred over-night. The mixture was cooled to room temperature and added to 150 mL of ethyl acetate. The mixture was washed three times with saturated NaCl aqueous solution (3×100 mL), and dried over anhydrous magnesium sulfate overnight. The crude product was purified by column chromatography on silica gel with 20% ethyl ether in hexane to provide 2.65 g (45.8%) of mono-tocopherol isophthalate.

Preparation of dl-α-tocopherol-isophthalate-camptothecin. A 100 mL flask was charged with 1.74 g (3 mmol) of mono-dl-α-tocopherol isophthalate, prepared as above, 1.05 g (3 mmol) of camptothecin, 0.77 g (3 mmol) of 2-chloro-1-methylpyridinium iodide, 0.73 g (6 mmol) of 4-dimethylaminopyridine, and 50 mL of N,N-dimethylacetamide. The mixture was stirred overnight at 50° C. under nitrogen. The reaction mixture was cooled to room temperature, and 200 mL of ethyl acetate was added to the mixture. The resulting mixture was filtered, washed with saturated NaCl aqueous solution (3×100 mL), and dried with anhydrous magnesium sulfate overnight. The crude product was purified by column chromatography on silica gel with 20% ethyl acetate in hexane and 30% acetone in hexane successively to provide 0.85 g (31.1%) of dl-α-tocopherol-isophthalate-camptothecin.

IRνₘₐₓ^{KBr} cm⁻¹: 3058, 2953, 2924, 2864, 1758, 1736, 1668, 1616, 1565, 1457, 1401, 1375, 1306, 1220, 1166, 1126, 1091, 724.

¹H NMR (300 MHz, CDCl₃) δ ppm: 8.991 (s, 1H), 8.510-8.484 (d, J=7.8 Hz, 1H), 8.381-8.350 (m, 2H), 8.162-8.134 (d, J=8.4, 1H), 7.934-7.908 (d, J=7.8 Hz, 1H), 7.820-7.769 (m, 1H), 7.690-7.622 (m, 2H), 7.289 (s, 1H), 5.822-5.460 (q, J₁=17.4 Hz, J₂=91.3 Hz, 2H), 5.296 (s, 2H), 2.629 (t, 2H), 2.530-2.311 (m, 2H), 2.128-2.041 (m, 9H), 1.870-1.772 (m, 2H), 1.678-1.079 (m, 27H), 0.874-0.837 (m, 12H).

### Example 29

### The Preparation of a Representative Tocopherol-Modified Camptothecin Compound: 7-Ethyl-10-(4-(d-α-tocopheryloxymethyl)benzyloxy)camptothecin

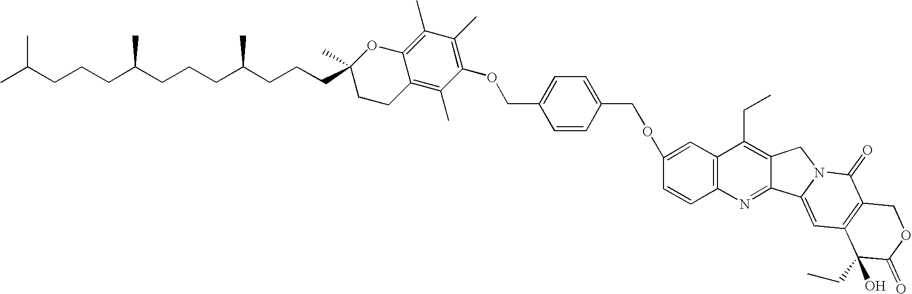

Preparation of 4-(bromomethyl)benzyl d-α-tocopherol ether. In a 200 mL flask, a mixture of d-α-tocopherol (4.30 g, 0.01 mol), α,α′-dibromo-ρ-xylene (5.20 g, 0.02 mol), cesium carbonate (4.0 g, 0.012 mol), and N,N-dimethylformamide (100 mL) was stirred for 4 hours at room temperature under nitrogen. To the mixture was added ethyl acetate (200 mL). The resulting mixture was washed with saturated NaCl aqueous solution three times (3×100 mL), and dried with anhydrous magnesium sulfate for 2 hours. The magnesium sulfate was removed by filtration and the solvent was removed under reduced pressure. The crude product was purified by column chromatography on silica gel with ethyl acetate and hexane to provide 4-(bromomethyl)benzyl d-α-tocopherol ether (2.26 g, 36.82%)

Preparation of 7-ethyl-10-(4-(d-α-tocopheryloxymethyl)benzyloxy)camptothecin. A mixture of 7-ethyl-10-hydroxycamptothecin (261 mg, 0.67 mmol), 4-(bromomethyl)benzyl d-α-tocopherol ether (957 mg, 1.56 mmol), prepared as above, cesium carbonate (350 mg, 1.07 mmol), and N,N-dimethylformamide (50 mL) was stirred overnight at room temperature under nitrogen. The mixture was filtered to remove the solid substance. The solvent was removed under reduced pressure. The crude product was purified by column chromatography on silica gel with dichloromethane, chloroform, and 0.5%-1% methanol in chloroform successively to provide 7-ethyl-10-(4-(d-α-tocopheryloxymethyl)benzyloxy)camptothecin (360 mg, 58.14%).

IRνₘₐₓ^{KBr} cm⁻¹: 3259.09, 2925.19, 1749.03, 1658.74, 1605.48, 1556.52, 1512.16, 1460.46, 1413.48, 1374.95, 1343.66, 1305.08, 1279.13, 1240.95, 1227.11, 1196.94, 1152.48, 1107.79, 1059.28, 1033.19, 998.83, 963.83, 944.14, 919.50, 864.03, 832.06, 804.97, 758.62, 724.42, 665.34.

MS (Positive ESI): m/z 925.8 (M+H)⁺.

### Example 30

### The Preparation of a Representative Tocopherol-Modified Camptothecin Compound: 20-Acetyl-10-(d-α-tocopherolsuccinate)-7-ethyl-10-hydroxycamptothecin

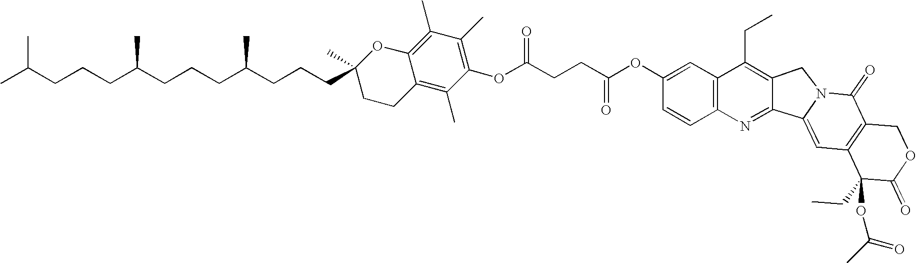

A 100 ml flask was charged with 0.905 g (1 mmol) of d-α-tocopherol succinate-7-ethyl-10-hydroxycamptothecin, prepared as in Example 3, 0.300 g (5 mmol) of acetic acid, 0.255 g (1 mmol) of 2-chloro-1-methylpyridinium iodide, 0.244 g (2 mmol) of 4-dimethylaminopyridine and 50 mL of dioxane. The mixture was stirred for 72 hours at room temperature under nitrogen. The mixture was filtered. The solvent was removed under reduced pressure. The crude product was purified by column chromatography on silica gel with 30% ethyl acetate in cyclohexane to provide 0.820 g (86.6%) of 20-acetyl-10-(d-α-tocopherolsuccinate)-7-ethyl-10-hydroxycamptothecin.

IRνₘₐₓ^{KBr} cm⁻¹: 2925, 2866, 1750, 1700, 1663, 1610, 1562, 1510, 1456, 1413, 1366, 1326, 1229, 1155, 1127, 1074, 1059, 1039, 1019, 983, 944, 923, 894, 830, 813, 758, 724, 670.

Elemental anal. calcd. for C₅₇H₇₄N₂O₁₀: C, 72.28; H, 7.87; N, 2.96. Found: C, 71.98; H, 7.86; N. 2.95.

### Example 31

### The Preparation of a Representative Tocopherol-Modified Camptothecin Compound: 10-Acetyl-20-(d-α-tocopherolsuccinate)-7-ethyl-10-hydroxycamptothecin

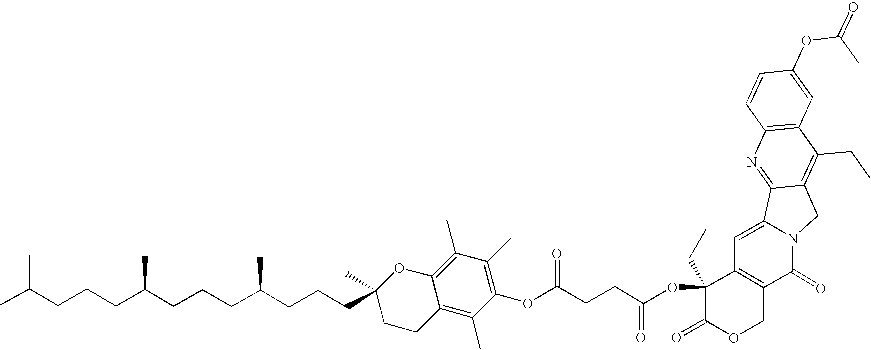

Preparation of 10-acetyloxy-7-ethylcamptothecin. A 100 mL flask was charged with 0.392 g (1 mmol) of 7-ethyl-10-hydroxcamptothecin and 30 mL of N,N-dimethylacetamide. To the solution was added 142 μL (0.156 g, 2 mmol) of acetyl chloride and 178 μL (0.202 g, 2 mmol) of triethylamine. The mixture was stirred overnight at room temperature under nitrogen, and then heated to 50° C. for other 4 hours. Thin Layer Chromatography showed that the reaction was complete. The mixture was concentrated to 10 mL by vacuum distillation with a rotavapor, and then poured to 100 mL of cold DI-water (0-5° C.) with stirring. A white precipitate was collected by filtration, and washed with cold DI-water. The product was dried in a vacuum oven at room temperature to provide 10-acetyloxy-7-ethylcamptothecin (0.353 g, yield 81.3%).

Preparation of 10-acetyl-20-(d-α-tocopherolsuccinate)-7-ethyl-10-hydroxycamptothecin. In a 100 mL flask, a mixture containing 0.43 g (1 mmol) of 10-acetyloxy-7-ethylcamptothecin, prepared as above, 0.78 g (1.5 mmol) of d-α-tocopherol acid succinate, 0.512 g (2 mmol) of 2-chloro-1-methylpyridinium iodide, 0.488 g (4 mmol) of 4-dimethylaminopyridine, and 30 mL of N,N-dimethylacetamide was stirred for 6 hours at room temperature under nitrogen. The reaction mixture was added to 200 mL of ethyl acetate with stirring. A precipitate was formed. The precipitate was removed by filtration. The filtrate was collected and washed with NaCl aqueous solution three times (3×100 mL), and dried with anhydrous magnesium sulfate for 1.5 hours. The magnesium sulfate was removed by filtration. The solvent was removed under reduced pressure. The crude product was purified by column chromatography on silica gel to provide 10-acetyl-20-(d-α-tocopherolsuccinate)-7-ethyl-10-hydroxycamptothecin (170 mg, 17.65%).

MS (Positive ESI): m/z 947.8 (M+H)⁺.

### Example 32

### The Preparation of a Representative Tocopherol-Modified Camptothecin Compound: δ-Tocopherolsuccinate-7-ethyl-10-hydroxycamptothecin

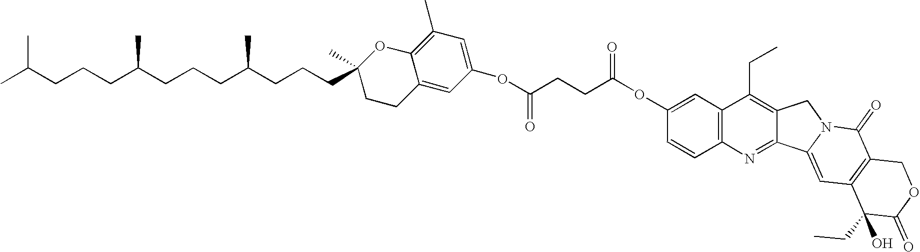

Preparation of δ-tocopherol succinic acid. A mixture containing 4.03 g of δ-tocopherol, 1.00 g of succinic acid anhydride, 100 mg of Tin (II) 2-ethylhexanoate, and 50 mL of xylene was stirred for 4 hours at refluxing temperature under nitrogen. The solvent, xylene, was removed under reduced pressure, and the crude product was purified by column chromatography on silica gel (acetone-hexane) to provide δ-tocopherol succinic acid (2.86 g, 56.9%).

Preparation of δ-tocopherolsuccinate-7-ethyl-10-hydroxycamptothecin. A mixture containing 502 mg (1 mmol) of δ-tocopherol succinic acid, prepared as above, 238 mg (2 mmol) of thionyl chloride, 50 mL of toluene and 100 μL of N,N-dimethylformamide was stirred overnight at room temperature under nitrogen. The toluene and excess thionyl chloride were removed by reduced pressure. To the residue was added 10 mL of chloroform to form Solution A. A mixture containing 196 mg (0.5 mmol) of 7-ethyl-10-hydroxycamptothecin and 100 mL of chloroform was stirred for 15 minutes to form Solution B. Solution A and 0.101 g (140 μL, 1 mmol) of triethylamine was added to Solution B. The resulting mixture was stirred overnight at room temperature under nitrogen. The mixture was filtered to remove solid substances. The solvent was removed under reduced pressure. The crude product was purified by column chromatography on silica gel (acetone-hexane) to provide δ-tocopherol succinate 7-ethyl-10-hydroxycamptothecin (183 mg, 41.72%).

IR νₘₐₓ (cm⁻¹): 3267.78, 2926.02, 2868.21, 1753.22, 1739.76, 1659.74, 1597.12, 1508.53, 1466.28, 1413.92, 1376.61, 1358.70, 1313.10, 1281.71, 1226.46, 1212.06, 1162.32, 1121.10, 1064.41, 1039.04, 1011.71, 986.79, 918.09, 895.59, 872.07, 848.28, 827.96, 810.04, 758.88, 738.72, 723.63, 691.67, 664.38.

MS (Positive ESI): m/z 877.7 (M+H)⁺.

¹H NMR (300 MHz, CDCl₃) δ ppm: 8.246-8.216 (d, J=9.0 Hz, 1H), 7.836-7.828 (d, J=2.4 Hz, 1H), 7.646 (s, 1H), 7.595-7.556 (dd, J=9.3 Hz & 2.4 Hz, 1H), 6.705-6.696 (d, J=2.7 Hz, 1H), 6.653-6.644 (d, J=2.7 Hz, 1H), 5.785-5.284 (q, J=16.2 & 134.1 Hz, 2H), 5.260 (s, 2H), 3.783 (s, 1H), 3.175-3.010 (m, 6H), 2.738-2.696 (t, J=6.3 Hz, 2H), 2.137 (s, 3H), 1.945-1.828 (m, 2H), 1.805-1.701 (m, 2H), 1.595-1.017 (m, 30H), 0.874-0.831 (m, 12H).

### Example 33

### The Preparation of a Representative Tocopherol-Modified Camptothecin Compound: δ-Tocopherolglutarate-7-ethyl-10-hydroxycamptothecin

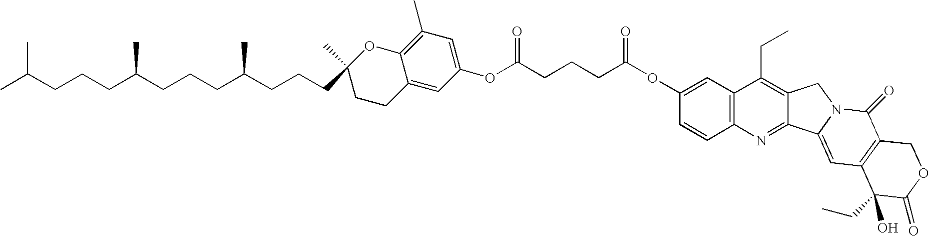

In a 200 mL flask, a mixture of 800 mg (1.55 mmol) of δ-tocopherol glutaric acid, 236 mg (2 mmol) thionyl chloride, 100 μL of N,N-dimethylformamide, and 100 mL of toluene was stirred for 4 hours at room temperature under nitrogen. The solvent and excess thionyl chloride were removed under reduced pressure and residue was collected. To the residue was added 196 mg (0.5 mmol) of 7-ethyl-10-hydroxycamptotehcin, 30 mL of N,N-dimethylacetamide, and 101 mg of triethylamine. The resulting mixture was stirred overnight at room temperature under nitrogen. To the mixture was added 200 mL of chloroform. The mixture was washed with deionized water three times (3×100 mL) and then dried with anhydrous magnesium sulfate for 2 hours. The magnesium sulfate was removed by filtration. The solvent was removed under reduced pressure. The crude product was purified with column chromatography on silica gel to provide 55.2 mg of δ-tocopherol glutarate 7-ethyl-10-hydroxycamptothecin.

IR νₘₐₓ (cm⁻¹): 3319.03, 2926.14, 2868.39, 1749.90, 1660.26, 1598.46, 1558.37, 1510.64, 1461.55, 1416.82, 1377.32, 1313.28, 1280.85, 1225.52, 1211.91, 1176.48, 1149.94, 1118.44, 1052.51, 1033.30, 998.48, 909.53, 833.79, 809.06, 788.44, 758.53, 723.60, 696.47, 665.61.

MS (Positive ESI): m/z 892.0 (M+H)⁺.

### Example 34

### The Preparation of a Representative Tocopherol-Modified Camptothecin Compound: δ-Tocopherol-3-methylglutarate-7-ethyl-10-hydroxycamptothecin

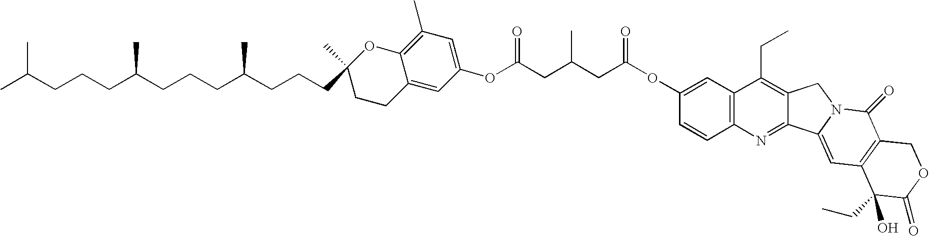

Preparation of δ-tocopherol-3-methylglutaric acid. A mixture containing 4.03 g (0.01 mol) of δ-tocopherol, 1.28 g (0.01 mol) of 3-methyl glutaric anhydride, 3.26 g (0.01 mol) of cesium carbonate, and 100 mL of dioxane was stirred for 4 hours at refluxing temperature under nitrogen. The mixture was filtered to remove solid substances, and the solvent was removed under reduced pressure. The crude product was purified by column chromatography on silica gel (acetone-hexane) to provide δ-tocopherol-3-methyl glutaric acid (3.12 g, 58.86%).

Preparation of δ-tocopherol-3-methylglutarate-7-ethyl-10-hydroxycamptothecin. A mixture containing 1.06 g (1 mmol) of δ-tocopherol-3-methylglutaric acid, prepared as above, 0.48 g (4 mmol) of thionyl chloride, 100 μL of N,N-dimethylformamide, and 100 mL of toluene was stirred for 4 hours at room temperature under reduced pressure. The solvent and excess thionyl chloride were removed under reduced pressure. The residue, δ-tocopherol succinic chloride, was collected and dissolved in 5 mL of chloroform to form Solution A. In a 100 mL flask, 0.392 g (1 mmol) of 7-ethyl-10-hydroxycamptothecin was dissolved in 30 mL of N,N-dimethylacetamide to form Solution B. Solution A and 0.101 g (139 μL, 1 mmol) of triethylamine were added to Solution B. The resulting mixture was stirred overnight at room temperature under nitrogen. The crude product was purified by column chromatography on silica gel (acetone-hexane) to provide δ-tocopherol-3-methylglutarate-7-ethyl-10-hydroxycamptothecin (118 mg, 13.0%).

IR νₘₐₓ (cm⁻¹): 3257.64, 2926.06, 2868.90, 1752.24, 1740.84, 1660.21, 1597.54, 1509.08, 1465.86, 1414.30, 1376.30, 1312.63, 1282.24, 1258.36, 1226.06, 1211.89, 1161.01, 1130.32, 1106.77, 1065.42, 1039.59, 1011.81, 993.35, 919.20, 866.20, 848.28, 830.43, 809.12, 759.07, 737.53, 722.65, 692.58, 664.83.

MS (Positive ESI): m/z 905.8 (M+H)⁺.

¹H NMR (300 MHz, CDCl₃) δ ppm: 8.255-8.225 (d, J=9.0 Hz, 1H), 7.838-7.829 (s, 1H), 7.654 (s, 1H), 7.590-7.551 (dd, J=9.3 Hz & 2.7 Hz, 1H), 6.683-6.674 (d, J=2.7 Hz, 1H), 6.630-6.621 (d, J=2.7 Hz, 1H), 5.789-5.287 (q, J=16.2 & 134.1 Hz, 1H), 5.265 (s, 2H), 3.762 (s, 1H), 3.169-3.092 (s, 2H), 2.872-2.604 (m, 6H), 2.123 (s, 3H), 1.947-1.852 (m, 2H), 1.818-1.693 (m, 2H), 1.588-1.020 (m, 33H), 0.874-0.830 (m, 12H).

### Example 35

### The Preparation of a Representative Tocopherol-Modified Camptothecin Compound: δ-Tocopherol-3,3-tetramethyleneglutarate-7-ethyl-10-hydroxycamptothecin

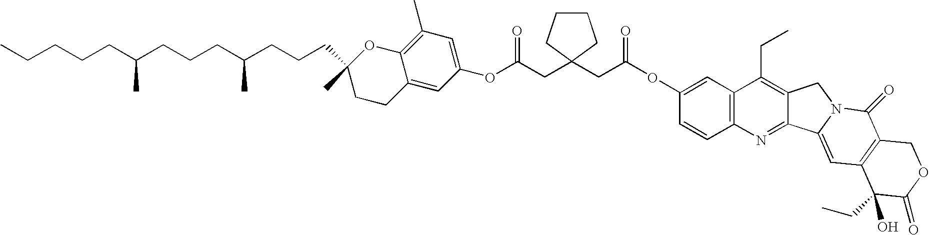

Preparation of δ-tocopherol-3,3-tetramethyleneglutaric acid. A mixture containing 4.03 g (0.01 mol) of δ-tocopherol, 1.68 g (0.01 mol) of 3,3-tetramethylene glutaric anhydride, 3.26 g (0.01 mol) of cesium carbonate, and 30 mL of N,N-dimethylformamide was stirred for 72 hours at room temperature under nitrogen. To the mixture was added to 150 mL of ethyl acetate with stirring. The mixture was washed with saturated NaCl aqueous solution three times (3×100 mL), and dried with anhydrous magnesium sulfate. The magnesium sulfate was removed by filtration. The ethyl acetate was removed under reduced pressure. The crude product was purified by column chromatography on silica gel to provide δ-tocopherol-3,3-tetramethyleneglutaric acid (3.851 g, 67.4%).

Preparation of δ-tocopherol-3,3-tetramethyleneglutarate-7-ethyl-10-hydroxycamptothecin. A mixture containing 168 mg (1 mmol) of δ-tocopherol-3,3-tetramethyleneglutaric acid, prepared as above, 236 mg (2 mmol) of thionyl chloride, 100 μL of N,N-dimethylformamide, and 100 mL of toluene was stirred for 4 hours at room temperature under nitrogen. The solvent and excess thionyl chloride were removed under reduced pressure. The residue was collected and dissolved in 5 mL of chloroform to form Solution A. In a 100 mL flask, 196 mg (0.5 mmol) of 7-ethyl-10-hydroxycamptothecin was dissolved in 30 mL of N,N-dimethylacetamide to form Solution B. Solution A and 101 mg (139 μL, 1 mmol) of triethylamine were added to Solution B. The resulting mixture was stirred overnight at room temperature under nitrogen. The crude product was purified by column chromatography on silica gel to provide δ-tocopherol-3,3-tetramethyleneglutarate-7-ethyl-10-hydroxycamptothecin (137 mg, 29.0%).

IR νₘₐₓ (cm⁻¹): 3257.11, 2926.14, 2869.30, 1739.38, 1659.63, 1597.40, 1509.07, 1465.34, 1416.02, 1375.96, 1313.04, 1282.42, 1258.04, 1225.96, 1211.95, 1160.55, 1131.37, 1066.27, 1036.02, 1012.42, 993.30, 927.27, 866.69, 848.56, 831.26, 809.21, 759.21, 722.71, 692.74, 665.40.

MS (Positive ESI): m/z 945.8 (M+H)⁺.

¹H NMR (300 MHz, CDCl₃) δ ppm: 8.245-8.214 (d, J=9.6 Hz, 1H), 7.806-7.798 (d, J=2.4 Hz, 1H), 7.649 (s, 1H), 7.592-7.554 (dd, J₁=9.0 Hz & J₂=3.0 Hz, 1H), 6.691-6.684 (d, J=2.1 Hz, 1H), 6.638-6.628 (d, J=3.0 Hz, 1H), 5.785-5.283 (q, J₁=134.11 Hz, J₂=16.2 Hz, 2H), 5.253 (s, 2H), 3.776 (2, 1H), 3.138-3.026 (q, J=7.8 Hz, 2H), 2.980 (s, 2H), 2.862 (s, 2H), 2.730-2.684 (t, J=7.5 Hz, 2H), 2.124 (s, 3H), 1.944-1.015 (m, 42H), 0.871-0.826 (m, 12H).

### Example 36

### The Preparation of a Representative Tocopherol-Modified Camptothecin Compound: δ-Tocopheroladipate-7-ethyl-10-hydroxycamptothecin

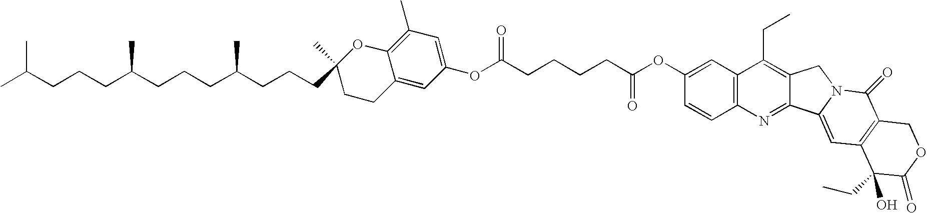

Preparation of δ-tocopherol adipic acid. In a 200 mL flask, a mixture of 4.02 g (10 mmol) of δ-tocopherol, 7.30 g (50 mmol) of adipic acid, 5.00 g (40.9 mmol) of 4-(dimethylamino)pyridine, 5.00 g (19.57 mmol) of 2-chloro-1-methylpyridinium iodide, and 100 mL of N,N-dimethylformamide was stirred for 4 hours at room temperature under nitrogen. To the mixture was added 200 mL of diethyl ether. The mixture was washed with saturated NaCl aqueous solution three times (3×100 mL), and dried with anhydrous magnesium sulfate for 2 hours. The magnesium sulfate was removed by filtration. The diethyl ether was removed under reduced pressure. The crude product was purified by column chromatography on silica gel to provide 2.278 g (42.12%) of δ-tocopherol adipic acid.

Preparation of δ-tocopheroladipate-7-ethyl-10-hydroxycamptothecin. In a 200 mL flask, a mixture of 800 mg (1.50 mmol) of δ-tocopherol adipic acid, prepared as above, 1305 mg (800 μL, 11 mmol) thionyl chloride, 100 μL of N,N-dimethylformamide, and 100 mL of toluene was stirred for 4 hours at room temperature under nitrogen. The solvent and excess thionyl chloride were removed under reduced pressure. The residue was collected and dissolved in 5 mL of chloroform to form Solution A. To Solution A was added 196 mg (0.5 mmol) of 7-ethyl-10-hydroxycamptothecin, 30 mL of N,N-dimethylacetamide, and 101 mg (1 mmol) of triethylamine. The mixture was stirred overnight at room temperature under nitrogen. After the reaction was complete, 200 mL of chloroform was added into the reaction mixture. The mixture was washed with deionized water three times (3×100 mL), and then dried with anhydrous magnesium sulfate for 2 hours. The magnesium sulfate was removed by filtration. The chloroform was removed under reduced pressure. The crude product was purified by column chromatography on silica gel to provide 235 mg (26%) of δ-tocopheroladipate-7-ethyl-10-hydroxycamptothecin.

IR νₘₐₓ (cm⁻¹): 3257.92, 2925.57, 2868.47, 1755.22, 1739.15, 1660.83, 1597.81, 1570.70, 1508.38, 1474.69, 1466.26, 1415.18, 1377.03, 1313.34, 1282.88, 1226.04, 1211.82, 1175.73, 1163.49, 1130.25, 1118.73, 1064.73, 1039.92, 1012.00, 970.55, 925.11, 864.51, 849.18, 831.67, 809.30, 759.01, 723.33, 664.73.

MS (Positive ESI): m/z 905.8 (M+H)⁺.

### Example 37

### The Preparation of a Representative Tocopherol-Modified Camptothecin Compound: δ-Tocopherolsebacate-7-ethyl-10-hydroxycamptothecin

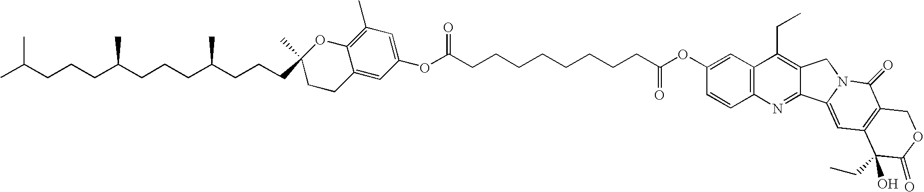

Preparation of δ-tocopherol sebacic acid. In a 200 mL flask, a mixture of 4.02 g (10 mmol) of δ-tocopherol, 10.00 g (49.4 mmol) of sebacic acid, 5.00 g (40.9 mmol) of 4-(dimethylamino)pyridine, 5.00 g (19.6 mmol) of 2-chloro-1-methylpyridinium iodide, and 100 mL of N,N-dimethylformamide was stirred for 4 hours at room temperature under nitrogen. To the mixture was added 200 mL of diethyl ether. The mixture was washed with saturated NaCl aqueous solution three times (3×100 mL), and then dried with anhydrous magnesium sulfate for 2 hours. The magnesium sulfate was removed by filtration. Diethyl ether was removed under reduced pressure. The crude product was purified by column chromatography on silica gel to provide 4.348 g (76.43%) of δ-tocopherol sebacic acid.

Preparation of δ-tocopherolsebacate-7-ethyl-10-hydroxycamptothecin. In a 200 mL flask, a mixture of 800 mg (1.36 mmol) of δ-tocopherol sebacic acid, prepared as above, 1305 mg (800 μL, 11 mmol) thionyl chloride, 100 μL of N,N-dimethylformamide, and 100 mL of toluene was stirred for 4 hours at room temperature under nitrogen. The solvent and excess thionyl chloride were removed under reduced pressure. To the residue was added 392 mg (1 mmol) of 7-ethyl-10-hydroxycamptothecin, 30 mL of N,N—dimethylacetamide, and 101 mg (1 mmol) of triethylamine. The mixture was stirred overnight at room temperature under nitrogen. To the mixture was added 200 mL of chloroform. The mixture was washed with deionized water three times (3×100 mL), and then dried with anhydrous magnesium sulfate for 2 hours. The magnesium sulfate was removed by filtration. The solvent was removed under reduced pressure. The crude product was purified with column chromatography on silica gel to provide 876 mg (91.15%) of δ-tocopherolsebacate-7-ethyl-10-hydroxycamptothecin.

IR νₘₐₓ (cm⁻¹): 3313.35, 3094.75, 2925.37, 2854.84, 1751.52, 1659.89, 1610.87, 1556.56, 1511.24, 1464.43, 1377.01, 1301.94, 1259.55, 1222.20, 1179.99, 1149.96, 1117.42, 1052.46, 1031.60, 999.51, 915.81, 868.16, 838.93.

MS (Positive ESI): m/z 962.0 (M+H)⁺.

### Example 38

### The Preparation of a Representative Tocopherol-Modified Camptothecin Compound: δ-Tocopherol suberate 7-ethyl-10-hydroxycamptothecin

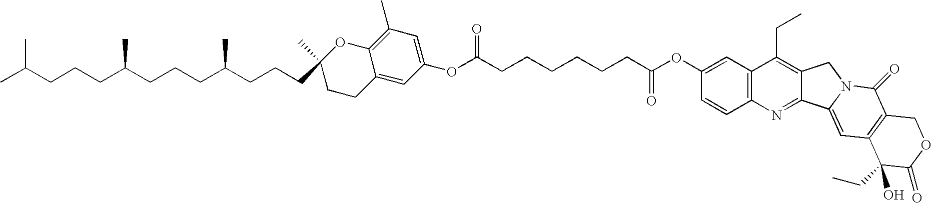

Preparation of δ-tocopherol suberic acid. In a 200 mL flask, a mixture of 4.30 g (10.7 mmol) of δ-tocopherol, 7.30 g (42.0 mmol) of suberic acid, 5.00 g (41.0 mmol) of 4-(dimethylamino)pyridine, 5.00 g (19.6 mmol) of 2-chloro-1-methylpyridinium iodide, and 100 mL of N,N-dimethylformamide was stirred for 4 hours at room temperature under nitrogen. To the mixture was added 200 mL of diethyl ether. The mixture was washed with saturated NaCl aqueous solution (3×100 mL), and then dried with anhydrous magnesium sulfate for 2 hours. The magnesium sulfate was removed by filtration. The solvent was removed under reduced pressure. The crude product was purified with column chromatography on silica gel to provide 2.73 g of δ-tocopherol suberic acid, yield 48.77%.

Preparation of δ-tocopherolsuberate-7-ethyl-10-hydroxycamptothecin. In a 200 mL flask, a mixture of 800 mg (1.43 mmol) of δ-tocopherol suberic acid, prepared as above, 236 mg (145 μL, 2 mmol) of thionyl chloride, 100 μL of N,N-dimethylformamide, 100 mL of toluene was stirred for 4 hours at room temperature under nitrogen. The solvent and excess thionyl chloride were removed under reduced pressure. To the residue was added 196 mg (0.5 mmol) of 7-ethyl-10-hydroxycamptothecin, 30 mL of N,N-dimethylacetamide, and 101 mg (1 mmol) of triethylamine. The mixture was stirred overnight at room temperature under nitrogen. 200 mL of chloroform was added to the mixture. The mixture was washed with deionized water (3×100 mL) and dried with anhydrous magnesium sulfate for 2 hours. The magnesium sulfate was removed by filtration, and the solvent was removed under reduced pressure. The crude product was purified with column chromatography on silica gel to provide 292 mg (62.66%) of δ-tocopherolsuberate-7-ethyl-10-hydroxycamptothecin.

IR νₘₐₓ (cm⁻¹): 3250.89, 2925.68, 2867.29, 1756.38, 1737.77, 1660.95, 1597.49, 1571.25, 1508.18, 1466.09, 1415.13, 1377.09, 1313.47, 1281.95, 1257.83, 1225.67, 1211.09, 1163.31, 1146.28, 1130.42, 1106.11, 1089.91, 1064.51, 1039.66, 1012.03, 970.02, 927.11, 864.99, 848.56, 828.44, 809.13, 758.95, 738.47, 723.31, 692.47, 665.00.

MS (Positive ESI): m/z 934.0 (M+H)⁺.

### Example 39

### The Preparation of a Representative Tocopherol-Modified Camptothecin Compound: δ-Tocopheroldodecanedioate-7-ethyl-10-hydroxycamptothecin

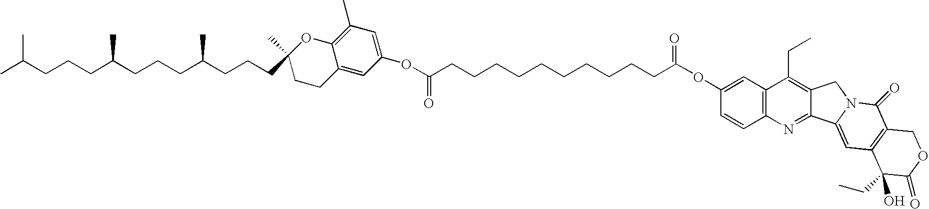

Preparation of δ-tocopherol dodecanedioic acid. In a 200 mL flask, a mixture of 4.02 g (10 mmol) of δ-tocopherol, 10.00 g (43.4 mmol) of dodecanedioic acid, 5.00 g (40.9 mmol) of 4-(dimethylamino)pyridine, 5.00 g (19.6 mmol) of 2-chloro-1-methyl pyridinium iodide, and 100 mL of N,N-dimethylformamide was stirred for 4 hours at room temperature under nitrogen. To the mixture was added 200 mL of diethyl ether. The mixture was washed with saturated NaCl aqueous solution (3×100 mL) and then dried with anhydrous magnesium sulfate for 2 hours. The magnesium sulfate was removed by filtration. The solvent was removed under reduced pressure. The crude product was purified with column chromatography on silica gel to provide 2.872 g (48.7%) of δ-tocopherol dodecanedioic acid.

Preparation of δ-tocopheroldodecanedioate-7-ethyl-10-hydroxycamptothecin. In a 200 mL flask, a mixture of 800 mg (1.3 mmol) of δ-tocopherol dodecanedioic acid, prepared as above, 392 mg (1 mmol) of 7-ethyl-10-hydroxycamptothecin, 500 mg (4.1 mmol) of 4-(dimethylamino)pyridine, 500 mg (2 mmol) of 2-chloro-1-methylpyridinium iodide, and 30 mL of N,N-dimethylacetamide was stirred overnight at room temperature under nitrogen. To the mixture was added 200 mL of chloroform. The mixture was washed with deionized water (3×100 mL), and then dried with anhydrous magnesium sulfate for 2 hours. The magnesium sulfate was removed by filtration and the solvent was removed under reduced pressure. The crude product was purified with column chromatography on silica gel to provide 497 mg (50.30%) of δ-tocopheroldodecanedioate-7-ethyl-10-hydroxycamptothecin.

IR νₘₐₓ (cm⁻¹): 3188.52, 2925.77, 2854.31, 1753.98, 1658.83, 1593.92, 1560.10, 1508.32, 1459.42, 1377.75, 1310.84, 1226.12, 1149.25, 1102.99, 1018.19, 927.62, 837.77, 803.76, 758.78, 722.39, 691.56, 671.12.

MS (Positive ESI): m/z 990.0 (M+H)⁺.

### Example 40

### The Preparation of a Representative Tocopherol-Modified Camptothecin Compound: δ-Tocopherol tetradecanedioate 7-ethyl-10-hydroxycamptothecin

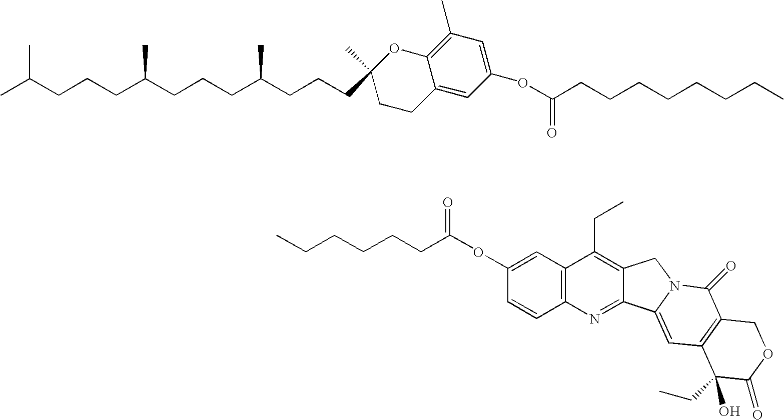

Preparation of δ-tocopherol tetradecanedioic acid. In a 200 mL flask, a mixture of 2.00 g (5 mmol) of δ-tocopherol, 4.00 g (15.5 mmol) of tetradecanedioic acid, 2.50 g (20.5 mmol) of 4-(dimethylamino)pyridine, 2.50 g (9.8 mmol) of 2-chloro-1-methyl pyridinium iodide, and 50 mL of N,N-dimethylformamide was stirred for 4 hours at room temperature under nitrogen. To the mixture was added 200 mL of diethyl ether. The mixture was washed with saturated NaCl aqueous solution (3×100 mL), and then dried with anhydrous magnesium sulfate for 2 hours. The magnesium sulfate was removed by filtration. The solvent was removed under reduced pressure. The crude product was purified by column chromatography on silica gel to provide 2.82 g (87.8%) of δ-tocopherol tetradecanedioic acid.

Preparation of δ-tocopheroltetradecanedioate-7-ethyl-10-hydroxycamptothecin. In a 200 mL flask, a mixture of 800 mg (1.24 mmol) of δ-tocopherol tetradecanedioic acid, prepared as above, 392 mg (1.00 mmol) of 7-ethyl-10-hydroxycamptothecin, 600 mg (4.92 mmol) of 4-(dimethylamino)pyridine, 600 mg (2.35 mmol) of 2-chloro-1-methylpyridinium iodide, and 30 mL of N,N-dimethylacetamide was stirred overnight at room temperature under nitrogen. 200 mL of chloroform was added into the mixture. The mixture was washed with deionized water (3×100 mL), and dried with anhydrous magnesium sulfate for 2 hours. The magnesium sulfate was removed by filtration. The solvent was removed under reduced pressure. The crude product was purified by column chromatography on silica gel to provide 120 mg (11.6%) of δ-tocopheroltetradecanedioate-7-ethyl-10-hydroxycamptothecin.

IR νₘₐₓ (cm⁻¹): 3187.15, 2924.00, 2853.00, 1758.98, 1660.79, 1634.50, 1595.36, 1566.57, 1505.41, 1460.88, 1377.10, 1355.57, 1328.54, 1309.16, 1293.10, 1249.34, 1224.69, 1206.43, 1145.80, 1102.50, 1076.80, 1058.05, 1040.09, 1018.57, 995.62, 951.12, 872.84, 854.77, 838.14, 827.67, 813.40, 783.79, 718.96, 671.17, 659.42.

MS (Positive ESI): m/z 1018.0 (M+H)⁺.

### Example 41

### The Preparation of a Representative Tocopherol-Modified Camptothecin Compound: dl-α-Tocopherolsuccinate-7-ethyl-10-hydroxycamptothecin

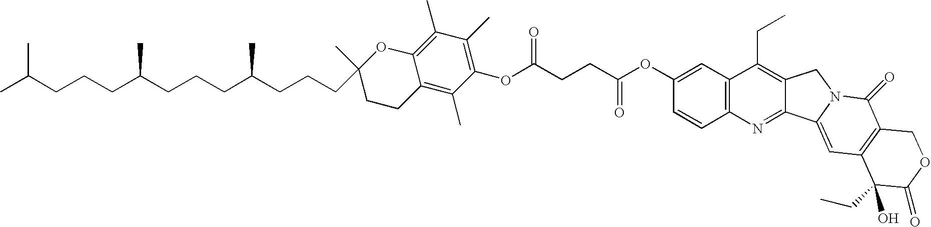

Preparation of dl-α-tocopherol succinic acid. A mixture containing 8.60 g (0.02 mol) of dl-α-tocopherol, 2.20 g (0.022 mol) of succinic acid anhydride, 100 mg of Tin(II) 2-ethylhexanoate, and 200 mL of xylene was stirred for 8 hours at 140° C. under nitrogen. The mixture was cooled down to room temperature and the solvent was removed under reduced pressure. To the residue was added 150 mL of ethyl ether. The mixture was cooled to about 0° C. and filtered to remove solid substances. The filtrate was concentrated by a rotavapor. The crude product was purified by column chromatography on silica gel with hexane-ethyl acetate to provide dl-α-tocopherol succinic acid (8.47 g, 79.8%).

Preparation of dl-α-tocopherolsuccinate-7-ethyl-10-hydroxycamptothecin. In a 200 mL flask, a mixture containing 530 mg (1 mmol) of dl-α-tocopherol succinic acid, prepared as above, 238 mg (2 mmol) of thionyl chloride, 50 mL of toluene, and 50 μL of N,N-dimethylformamide was stirred for 6 hours at room temperature under nitrogen. The solvent and excess thionyl chloride were removed under reduced pressure, and the residue was collected. To the residue was added 10 mL of dichloromethane, 196 mg (0.5 mmol) of 7-ethyl-10-hydroxycamptothecin, 30 mL of N,N-dimethylformamide, and 202 mg (2 mmol) of triethylamine. The mixture was stirred overnight at room temperature under nitrogen. 100 mL of ethyl acetate was added. The resulting mixture was washed with saturated NaCl aqueous solution three times (3×100 mL), and dried with anhydrous magnesium sulfate. The magnesium sulfate was removed by filtration. The filtrate was concentrated to about 10 mL. The crude product was then purified by column chromatography on silica gel with ethyl acetate-hexane to provide dl-α-tocopherolsuccinate-7-ethyl-10-hydroxycamptothecin (246 mg, 54.3%).

IR νₘₐₓ (cm⁻¹): 3253.36, 2925.53, 2868.38, 1740.29, 1660.70, 1597.97, 1508.63, 1460.63, 1414.21, 1376.80, 1331.50, 1313.82, 1282.16, 1226.84, 1212.16, 1175.68, 1162.72, 1127.71, 1106.94, 1064.27, 1039.48, 1012.54, 918.38, 848.97, 829.26, 809.91, 758.91, 738.68, 723.68, 665.08.

MS (Positive ESI): m/z 905.8 (M+H)⁺.

### Example 42

### The Preparation of a Representative Tocopherol-Modified Camptothecin Compound: dl-α-Tocopherolglutarate-7-ethyl-10-hydroxycamptothecin

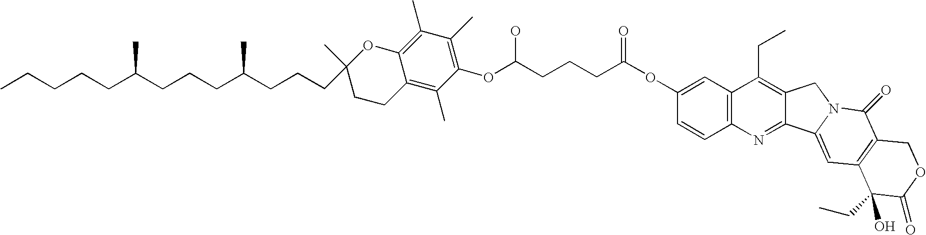

Preparation of dl-α-tocopherol glutaric acid. A mixture containing 14.00 g (0.033 mol) of dl-α-tocopherol, 4.30 g (0.036 mol) of glutaric anhydride, 100 mg of Tin(II) 2-ethylhexanoate, and 200 mL of xylene was stirred for 8 hours at 140° C. under nitrogen. The mixture was cooled down to room temperature and filtered to remove solid substances. The filtrate was collected and the solvent was removed under reduced pressure. The crude product was purified by column chromatography on silica gel with hexane-ethyl acetate to provide dl-α-tocopherol glutaric acid (12.10 g, 67.07%).

Preparation of dl-α-tocopherolglutarate-7-ethyl-10-hydroxycamptothecin. In a 200 mL flask, 1.00 g (1.84 mmol) of dl-α-tocopherol glutaric acid, prepared as above, 1.30 g (800 μL, 11 mmol) of thionyl chloride, 100 μL of N,N-dimethylformamide, and 100 mL of toluene was stirred for 4 hours at room temperature under nitrogen. The solvent and excess thionyl chloride were removed under reduced pressure. To the residue was added to 196 mg (0.5 mmol) of 7-ethyl-10-hydroxycamptothecin, 30 mL of N,N-dimethylacetamide, and 101 mg of triethylamine. The mixture was stirred overnight at room temperature under nitrogen. 200 mL of ethyl acetate was added into the reaction mixture with stirring. The resulting mixture was washed with saturated NaCl aqueous solution (3×100 mL). The ethyl acetate portion was collected and dried with anhydrous magnesium sulfate for 2 hours. The solvent was removed under reduced pressure. The crude product was purified by column chromatography on silica gel to provide 190 mg (41.48%) of dl-α-tocopherolglutarate-7-ethyl-10-hydroxycamptothecin.

IR ν_{MAX} (CM⁻¹): 3253.59, 2925.43, 2868.61, 1740.59, 1661.03, 1598.42, 1508.55, 1462.35, 1414.52, 1376.87, 1313.63, 1281.80, 1226.61, 1211.95, 1175.64, 1150.71, 1125.59, 1107.16, 1063.74, 1039.38, 1012.37, 997.77, 965.92, 918.38, 862.22, 848.26, 830.07, 809.50, 758.92, 738.79, 723.68, 665.01.

MS (Positive ESI): m/z 919.8 (M+H)⁺.

### Example 43

### The Preparation of a Representative Tocopherol-Modified Camptothecin Compound: dl-α-Tocopheroladipate-7-ethyl-10-hydroxycamptothecin

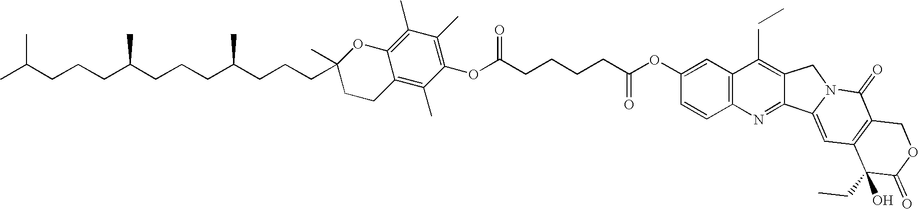

Preparation of dl-α-tocopherol adipic acid. In a 200 mL flask, a mixture containing 4.30 g (0.01 mol of dl-α-tocopherol, 7.31 g (0.05 mol) of adipic acid, 2.44 g (0.02 mol) of 4-(diemthylamino)pyrindine, 2.55 g (0.01 mol) of 2-chloro-1-methylpyridinium iodide, and 100 mL of N,N-dimethylformamide was stirred for 4 hours at room temperature under nitrogen. To the mixture was added to 200 mL of diethyl ether. The mixture was washed with saturated NaCl aqueous solution three times (3×100 mL). The diethyl ether portion was collected and dried with anhydrous magnesium sulfate for 2 hours. The crude product was purified by column chromatography on silica gel to provide 4.80 g (85.83%) of dl-α-tocopherol adipic acid.

Preparation of dl-α-tocopheroladipate-7-ethyl-10-hydroxycamptothecin. In a 200 mL flask, 800 mg (1.43 mmol) of dl-α-tocopherol adipic acid, prepared as above, 1305 mg (800 μL, 11 mmol) of thionyl chloride, 100 μL of N,N-dimethylformamide, and 100 mL of toluene was stirred for 4 hours at room temperature under nitrogen. The solvent and excess thionyl chloride were removed under reduced pressure. To the residue was added 196 mg (0.5 mmol) of 7-ethyl-10-hydroxycamptothecin, 30 mL of N,N-dimethylacetamide, and 101 mg of triethylamine. The mixture was stirred overnight at room temperature under nitrogen. 200 mL of ethyl acetate was added into the reaction mixture. The resulting mixture was washed with saturated NaCl aqueous solution three times (3×100 mL). The ethyl acetate portion was collected and dried with anhydrous magnesium sulfate for 2 hours. The crude product was then purified with column chromatography on silica gel to provide 198 mg (44.5%) of dl-α-tocopheroladipate-7-ethyl-10-hydroxycamptothecin.

IR νₘₐₓ (cm⁻¹): 3256.04, 2926.18, 2857.00, 1739.61, 1660.93, 1597.92, 1508.67, 1463.81, 1414.44, 1376.55, 1282.91, 1226.25, 1211.51, 1162.54, 1130.79, 1106.48, 1064.36, 1039.85, 1012.47, 922.44, 848.44, 828.02, 809.99, 758.95, 723.48, 665.03.

MS (Positive ESI): m/z 934.0 (M+H)⁺.

### Example 44

### The Preparation of a Representative Tocopherol-Modified Camptothecin Compound: dl-α-Tocopherolsuberate-7-ethyl-10-hydroxycamptothecin

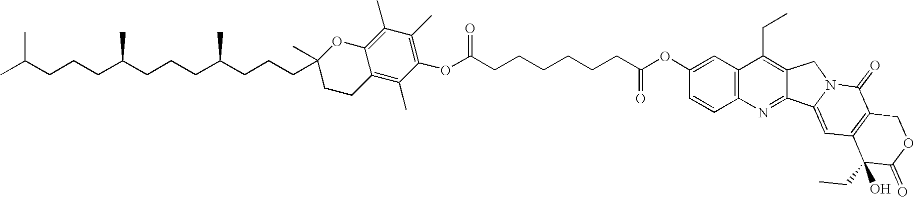

Preparation of dl-α-tocopherol suberic acid. In a 200 mL flask, 4.3 g (10 mmol) of dl-α-tocopherol, 7.3 g (42 mmol) of suberic acid, 5.0 g (40.9 mmol) of 4-(dimethylamino)pyridine, 5.0 g (19.57 mmol) of 2-chloro-1-methylpyridinium iodide, and 100 mL of N,N-dimethylformamide was stirred for 4 hours at room temperature under nitrogen. To the mixture was added 200 mL of diethyl ether with stirring. The mixture was washed with saturated NaCl aqueous solution (3×100 mL), and then dried with anhydrous magnesium sulfate for 2 hours. The magnesium sulfate was removed by filtration and the solvent was removed under reduced pressure. The crude product was purified with column chromatography on silica gel to provide 5.15 g (87.68%) of dl-α-tocopherol suberic acid.

Preparation of dl-α-tocopherolsuberate-7-ethyl-10-hydroxycamptothecin. In a 200 mL flask, a mixture of 800 mg (1.37 mmol) of dl-α-tocopherol suberic acid, 236 mg (145 μL, 2 mmol) of thionyl chloride, 100 μL of N,N-dimethylformamide, and 100 mL of toluene was stirred for 4 hours at room temperature under nitrogen. The solvent and excess thionyl chloride were removed under reduced pressure. To the residue was added 196 mg (0.5 mmol) of 7-ethyl-10-hydroxycamptothecin, 30 mL of N,N-dimethylacetamide, and 101 mg (1 mmol) of triethylamine. The mixture was stirred overnight at room temperature under nitrogen. To the mixture was added 200 mL of ethyl acetate. The mixture was washed with saturated NaCl aqueous solution (3×100 mL). The ethyl acetate portion was collected and dried with anhydrous magnesium sulfate for 2 hours. The magnesium sulfate was removed by filtration. The solvent was then removed by a rotavapor. The crude product was purified by column chromatography on silica gel to provide 169 mg (36.26%) of dl-α-tocopherolsuberate-7-ethyl-10-hydroxycamptothecin.

IR νₘₐₓ (cm⁻¹): 3258.06, 2925.70, 2861.97, 1749.18, 1660.30, 1598.47, 1510.67, 1460.16, 1415.03, 1376.78, 1330.10, 1226.25, 1153.06, 1107.61, 1032.96, 997.54, 916.99, 835.51, 809.37, 724.26, 667.25.

MS (Positive ESI): m/z 961.8 (M+H)⁺.

### Example 45

### The Preparation of a Representative Tocopherol-Modified Camptothecin Compound: dl-α-Tocopherolsebacate-7-ethyl-10-hydroxycamptothecin

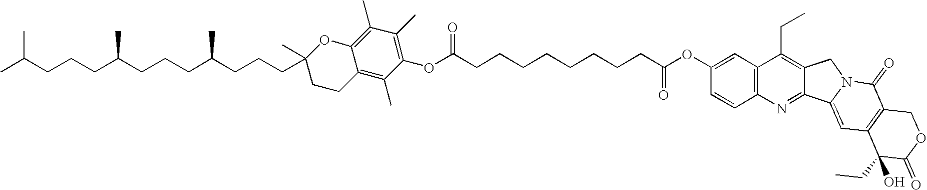

Preparation of dl-α-tocopherol sebacic acid. In a 200 mL flask, a mixture of 4.3 g (10 mmol) of dl-α-tocopherol, 10.0 g (49.4 mmol) of sebacic acid, 5.0 g (40.9 mmol) of 4-(dimethylamino)pyridine, 5.0 g (19.6 mmol) of 2-chloro-1-methylpyridinium iodide, and 100 mL of N,N-dimethylformamide was stirred for 4 hours at room temperature under nitrogen. To the mixture was added 200 mL of diethyl ether. The mixture was washed with saturated NaCl aqueous solution (3×100 mL), and then dried with anhydrous magnesium sulfate for 2 hours. The magnesium sulfate was removed by filtration and the solvent was removed under reduced pressure. The crude product was purified by column chromatography on silica gel to provide 3.664 g (59.6%) of dl-α-tocopherol sebacic acid.

Preparation of dl-α-tocopherolsebacate-7-ethyl-10-hydroxycamptothecin. In a 200 mL flask, a mixture of 800 mg (1.24 mmol) of dl-α-tocopherol sebacic acid, 1305 mg (800 μL, 11 mmol) of thionyl chloride, 100 μL of N,N-dimethylformamide, and 100 mL of toluene was stirred for 4 hours at room temperature under nitrogen. The solvent and excess thionyl chloride were removed under reduced pressure. To the flask was added 196 mg (0.5 mmol) of 7-ethyl-10-hydroxycamptothecin, 30 mL of N,N-dimethylacetamide, and 101 mg (1 mmol) of triethylamine. The mixture was stirred overnight at room temperature under nitrogen. To the reaction mixture was added 200 mL of ethyl acetate. The mixture was washed with saturated NaCl aqueous solution (3×100 mL), and then dried with anhydrous magnesium sulfate for 2 hours. The magnesium sulfate was removed by filtration. The ethyl acetate was removed under reduced pressure. The crude product was purified by column chromatography on silica gel to provide 293 mg (50.2%) of dl-α-tocopherolsebacate-7-ethyl-10-hydroxycamptothecin.

IR νₘₐₓ (cm⁻¹): 3256.04, 2926.18, 2857.00, 2166.33, 1739.61, 1660.93, 1597.92, 1508.67, 1463.81, 1414.44, 1376.55, 1282.91, 1226.25, 1211.51, 1162.54, 1130.79, 1106.48, 1064.36, 1039.85, 1012.47, 922.44, 848.44, 828.02, 809.99, 758.95, 723.48, 665.03.

MS (Positive ESI): m/z 990.0 (M+H)⁺.

### Example 46

### The Preparation of a Representative Tocopherol-Modified Camptothecin Compound: dl-α-Tocopheroldodecanedioate-7-ethyl-10-hydroxycamptothecin

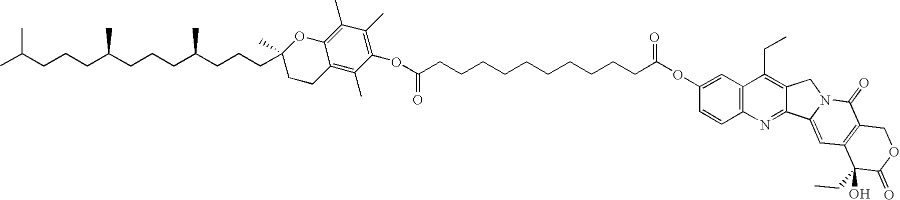

Preparation of dl-α-tocopherol dodecanedioic acid. In a 200 mL flask, a mixture of 4.3 g (10 mmol) of dl-α-tocopherol, 11.5 g (50 mmol) of dodecanedioic acid, 5.0 g (40.9 mmol) of 4-(dimethylamino)pyridine, 5.0 g (19.57 mmol) of 2-chloro-1-methylpyridinium iodide, and 100 mL of N,N-dimethylformamide was stirred over night at room temperature under nitrogen. To the reaction mixture was added 200 mL of diethyl ether. The mixture was washed with saturated NaCl aqueous solution three times (3×100 mL), and then dried with anhydrous magnesium sulfate for 2 hours. The magnesium sulfate was removed by filtration. The solvent was removed under reduced pressure. The crude product was purified by column chromatography on silica gel to provide 3.94 g (61.34%) of dl-α-tocopherol dodecanedioic acid.

Preparation of dl-α-tocopheroldodecanedioate-7-ethyl-10-hydroxycamptothecin. In a 200 mL flask, a mixture of 800 mg (1.24 mmol) of dl-α-tocopherol dodecanedioic acid, prepared as above, 392 mg (1 mmol) of 7-ethyl-10-hydroxycamptothecin, 500 mg (4.10 mmol) of 4-(dimethylamino)pyridine, 500 mg (2.00 mmol) of 2-chloro-1-methylpyridinium iodide, and 30 mL of N,N-dimethylformamide was stirred overnight at room temperature under nitrogen. To the mixture was added 200 mL of chloroform. The mixture was washed with deionized water three times (3×100 mL), and dried with anhydrous magnesium sulfate for 2 hours. The magnesium sulfate was removed by filtration. The solvent was removed under reduced pressure by a rotavapor. The crude product was purified by column chromatography on silica gel to provide 153 mg (15.10%) of dl-α-tocopheroldodecanedioate-7-ethyl-10-hydroxycamptothecin.

IR νₘₐₓ (cm⁻¹): 3214.85, 2924.70, 2854.07, 1755.29, 1659.22, 1635.09, 1596.20, 1566.67, 1507.79, 1459.72, 1415.46, 1377.07, 1329.41, 1309.28, 1292.36, 1248.39, 1225.65, 1206.13, 1146.46, 1100.78, 1076.13, 1058.46, 998.61, 950.43, 923.75, 877.28, 855.24, 836.47, 813.66, 782.81, 713.21, 658.15.

MS (Positive ESI): m/z 1018.0 (M+H)⁺.

### Example 47

### The Preparation of a Representative Tocopherol-Modified Camptothecin Compound: dl-α-Tocopheroltetradecanedioate-7-ethyl-10-hydroxycamptothecin

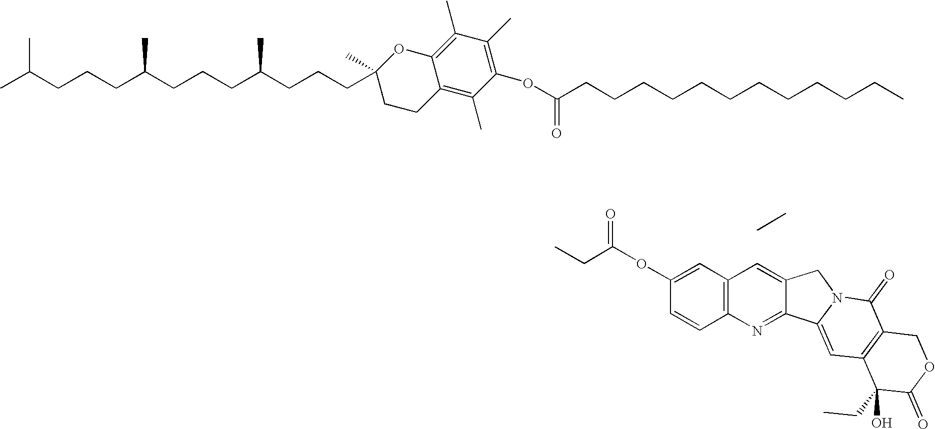

Preparation of dl-α-tocopherol tetradecanedioic acid. In a 200 mL flask, a mixture of 2.0 g (4.65 mmol) of dl-α-tocopherol, 2.5 g (10 mmol) of tetradecanedioic acid, 2.5 g (20.5 mmol) of 4-(dimethylamino)pyridine, 2.5 g (9.76 mmol) of 2-chloro-1-methylpyridinium iodide, and 50 mL of N,N-dimethylformamide was stirred for 4 hours at room temperature under nitrogen. To the reaction mixture was added 200 mL of diethyl ether. The mixture was washed with saturated NaCl aqueous solution three times (3×100 mL), and then dried with anhydrous magnesium sulfate for 2 hours. The magnesium sulfate was removed by filtration. The solvent was removed under reduced pressure. The crude product was purified by column chromatography on silica gel to provide 1.963 g (40.4%) of dl-α-tocopherol tetradecanedioic acid.

Preparation of dl-α-tocopheroltetradecanedioate-7-ethyl-10-hydroxycamptothecin. In a 200 mL flask, a mixture of 800 mg (1.2 mmol) of dl-α-tocopherol tetradecanedioic acid, prepared as above, 392 mg (1 mmol) of 7-ethyl-10-hydroxycamptothecin, 500 mg (4.10 mmol) of 4-(dimethylamino)pyridine, 500 mg (2.00 mmol) of 2-chloro-1-methyl pyridinium iodide, and 30 mL of N,N-dimethylformamide was stirred overnight at room temperature under nitrogen. To the mixture was added 200 mL of chloroform. The mixture was washed with deionized water three times (3×100 mL), and then dried with anhydrous magnesium sulfate for 2 hours. The magnesium sulfate was removed by filtration. The solvent was removed under reduced pressure by a rotavapor. The crude product was purified by column chromatography on silica gel to provide 405 mg (38.8%) of dl-α-tocopheroltetradecanedioate-7-ethyl-10-hydroxycamptothecin.

IR νₘₐₓ (cm⁻¹): 3622.34, 3086.19, 2924.55, 2852.96, 2753.74, 2650.94, 1747.17, 1659.92, 1635.56, 1592.30, 1561.44, 1506.83, 1460.95, 1415.93, 1377.17, 1358.85, 1329.87, 1308.86, 1249.92, 1206.90, 1150.97, 1102.74, 1086.71, 1063.30, 1036.77, 990.73, 950.47, 843.45, 825.90, 816.40, 787.53, 753.97, 721.52.

MS (Positive ESI): m/z 1046.1 (M+H)⁺.

### Example 48

### In Vitro Cytotoxicity of Representative Tocopherol-Modified Therapeutic Drug Compounds

In this example, the in vitro cytotoxicity, as measured by GI₅₀ (50% of growth inhibition) values, of representative tocopherol-modified therapeutic drug compounds of the invention was measured in the following cell lines: NCI-H460 (ATCC #HTB-177) (non-small cell lung), HCT-15 (ATCC #CCL-225) (colorectal), HT-116 (ATCC #CCL-247) (colorectal), HT-29 (ATCC #HTB-38) (colorectal), and SKOV-3 (ATCC #HTB-77) (ovarian). The study was performed using either Alamar Blue or Sulfarhodamine B assays. The results are shown in Table 5.

TABLE 5
Drug concentration that produced 50% cell growth inhibition (GI₅₀).

| | Cell line | | | | |
|---|---|---|---|---|---|
| | H460 | HCT-15 | HCT-116 | SKOV-3 | HT-29 |
| Compound | (NSCLC) | (COLON) | (COLON) | (OVARIAN) | (COLON) |
| VESA-10-HO-CPT | 155 nM | 1.29 μM | 186 nM | 97.7 nM | |
| 10,20-diVESA-SN38 | 4.06 μM | | 9.33 μM | | |
| 20-acetyl-10-VESA-SN38 | 1.26 μM | | 144 nM | | |
| 10-acetyl-20-VESA-SN38 | 3.9 μM | | | | |
| d-α-Tocopherolglutarate-SN38 | | | 190 nM | | |
| d-α-tocopherol-3- | | | 116 nM | | |
| methylglutarate-SN38 | | | 187 nM | | |
| d-α- | 560 nM | | 214 nM | | 48 nM |
| tocopheroltetramethyleneglutarate- | | | | | |
| SN38 | | | | | |
| δ-tocopherol succinate-SN38 | | | | 2.95 nM | |
| δ-tocopherol-3-methylglutarate- | | | | 1.48 nM | |
| SN38 | | | | | |

VESA: d-α-tocopherol succinic acid;
10-HO-CPT: 10-hydroxycamptothecin;
SN38: 7-ethyl-10-hydroxycamptothecin;
VESA-10-HO—CPT: d-α-tocopherol succinate-10-hydroxycamptothecin;
20-acetyl-10-VESA-SN38: 20-acetyl-10-(d-α-tocopherolsuccinate)-7-ethyl-10-hydroxycamptothecin;
10-acetyl-20-VESA-SN38: 10-acetyl-20-(d-α-tocopherolsuccinate)-7-ethyl-10-hydroxycamptothecin;
d-α-tocopherolglutarate-SN38: d-α-tocopherolglutarate-7-ethyl-10-hydroxycamptothecin;
d-α-tocopherol-3-methylglutarate-SN38: d-α-tocopherol-3-methylglutarate-7-ethyl-10-hydroxycamptothecin;
d-α-tocopheroltetramethyleneglutarate-SN38: d-α-tocopheroltetramethyleneglutarate-7-ethyl-10-hydroxycamptothecin;
δ-tocopherol succinate-SN38: 6-tocopherolsuccinate-7-ethyl-10-hydroxycamptothecin;
δ-tocopherol-3-methylglutarate-SN38: 6-tocopherol-3-methylglutarate-7-ethyl-10-hydroxycamptothecin.

While illustrative embodiments have been illustrated and described, it will be appreciated that various changes can be made therein without departing from the spirit and scope of the invention.

The embodiments of the invention in which an exclusive property or privilege is claimed are defined as follows:

1. A compound having the formula:
T-O—C(═O)—R—C(═O)—O-D
wherein
T is a tocopherol moiety derived from a tocopherol selected from the group consisting of:
(a) α-tocopherol,
(b) β-tocopherol,
(c) γ-tocopherol,
(d) δ-tocopherol,
(e) α-tocotrienol,
(f) β-tocotrienol,
(g) γ-tocotrienol, and
(h) δ-tocotrienol;
D is a therapeutic drug moiety derived from a therapeutic drug selected from the group consisting of:
(a) paclitaxel and its derivatives,
(b) docetaxel and its derivatives, and
(c) camptothecin and its derivatives; and
R is a divalent moiety selected from the group consisting of:
(a) substituted and unsubstituted alkyl,
(b) substituted and unsubstituted cycloalkyl, and
(c) substituted and unsubstituted aryl.

2. The compound of claim 1, wherein the tocopherol moiety is selected from the group consisting of an α-tocopherol moiety and a δ-tocopherol moiety.

3. The compound of claim 1, wherein the tocopherol moiety is a dl-α-tocopherol moiety.

4. The compound of claim 1, wherein the therapeutic drug moiety is selected from the group consisting of a 10-hydroxycamptothecin moiety and a 7-ethyl-10-hydroxycamptothecin moiety.

5. The compound of claim 1, wherein the divalent moiety is substituted and unsubstituted alkyl.

6. A compound having the formula 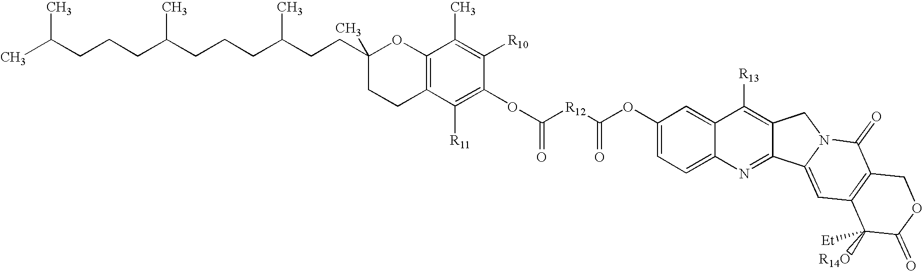
or a stereoisomer, tautomer, or pharmaceutically acceptable salt thereof, wherein
R₁₀ is hydrogen or methyl;
R₁₁ is hydrogen or methyl;
R₁₂ is selected from the group consisting of:
(a) —(CH₂)ₙ—, where n is an integer from 3 to 12;
(b) —CH₂—CH(CH₃)—CH₂—;
(c) —CH₂—C(CH₂)ₘ—CH₂—, where m is an integer from 2 to 6;
(d) 1,3-phenylene;
(e) 1,4-phenylene;
(f) 1,2-cycloalkylene;
(g) 1,3-cycloalkylene; and
(h) 1,4-cycloalkylene;
R₁₃ is hydrogen or ethyl; and
R₁₄ is selected from the group consisting of hydrogen and —C(═O)—R₁₅, where R₁₅ is C1-C6 alkyl.

7. The compound of claim 6, wherein R₁₀ and R₁₁ are methyl.

8. The compound of claim 6, wherein R₁₀ and R₁₁ are hydrogen.

9. The compound of claim 6, wherein R₁₂ is —(CH₂)ₙ— and n is an integer from 3 to 12.

10. The compound of claim 6, wherein R₁₂ is —(CH₂)ₙ— and n is 3.

11. The compound of claim 6, wherein R₁₃ is ethyl.

12. The compound of claim 6, wherein R₁₄ is hydrogen.

13. The compound of claim 6, wherein the tocopherol moiety is a dl-α-tocopherol moiety.

14. A compound having the formula 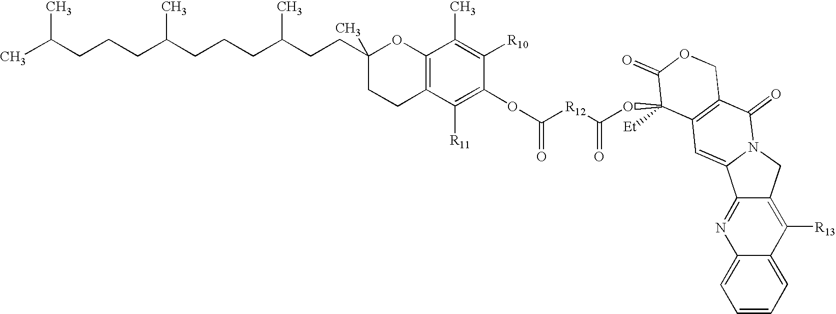
or a stereoisomer, tautomer, or pharmaceutically acceptable salt thereof, wherein
R₁₀ is hydrogen or methyl;
R₁₁ is hydrogen or methyl;
R₁₂ is selected from the group consisting of:
(a) —(CH₂)ₙ—, where n is an integer from 3 to 12;
(b) —CH₂—CH(CH₃)—CH₂—;
(c) —CH₂—C(CH₂)ₘ—CH₂—, where m is an integer from 2 to 6;
(d) 1,3-phenylene;
(e) 1,4-phenylene;
(f) 1,2-cycloalkylene;
(g) 1,3-cycloalkylene; and
(h) 1,4-cycloalkylene; and
R₁₃ is hydrogen or ethyl.

15. The compound of claim 14, wherein R₁₀ and R₁₁ are methyl.

16. The compound of claim 14, wherein R₁₀ and R₁₁ are hydrogen.

17. The compound of claim 14, wherein R₁₂ is —(CH₂)ₙ— and n is an integer from 3 to 12.

18. The compound of claim 14, wherein R₁₂ is —(CH₂)ₙ— and n is 3.

19. The compound of claim 14, wherein R₁₃ is hydrogen.

20. The compound of claim 14, wherein the tocopherol moiety is a dl-α-tocopherol moiety.

21. A pharmaceutical composition, comprising a pharmaceutically acceptable carrier and the compound of claim 1.

22. A method for treating a solid tumor carcinoma, comprising administering to a subject in need thereof a therapeutically effective amount of the compound of claim 1.

23. A method for treating a solid tumor carcinoma, comprising administering to a subject in need thereof an emulsion comprising:
(a) an oil phase comprising
(i) the compound of claim 1 and
(ii) a lipophilic medium; and
(b) an aqueous phase.

24. The method of claims 22 or 23, wherein the solid tumor carcinoma is selected from breast, ovarian, pancreatic, colon, colorectal, non-small cell lung, and bladder carcinomas.

25. The method of claim 23, wherein the lipophilic medium comprises a tocopherol.

26. A pharmaceutical composition, comprising a pharmaceutically acceptable carrier and the compound of claim 6.

27. A method for treating a solid tumor carcinoma, comprising administering to a subject in need thereof a therapeutically effective amount of the compound of claim 6.

28. A method for treating a solid tumor carcinoma, comprising administering to a subject in need thereof an emulsion comprising:
(a) an oil phase comprising
(i) the compound of claim 6 and
(ii) a lipophilic medium; and
(b) an aqueous phase.

29. The method of claims 27 or 28, wherein the solid tumor carcinoma is selected from breast, ovarian, pancreatic, colon, colorectal, non-small cell lung, and bladder carcinomas.

30. The method of claim 28, wherein the lipophilic medium comprises a tocopherol.

31. A pharmaceutical composition, comprising a pharmaceutically acceptable carrier and the compound of claim 14.

32. A method for treating a solid tumor carcinoma, comprising administering to a subject in need thereof a therapeutically effective amount of the compound of claim 14.

33. A method for treating a solid tumor carcinoma, comprising administering to a subject in need thereof an emulsion comprising:
(a) an oil phase comprising
(i) the compound of claim 14 and
(ii) a lipophilic medium; and
(b) an aqueous phase.

34. The method of claims 32 or 33, wherein the solid tumor carcinoma is selected from breast, ovarian, pancreatic, colon, colorectal, non-small cell lung, and bladder carcinomas.

35. The method of claim 33, wherein the lipophilic medium comprises a tocopherol.
